SEQUENCE LISTING (16 sequences)

SEQ ID NO: 1 - protein, 29 aa, Artificial Sequence
  Chemically Synthesized
YXDAIFTXXXRKVLXQLSARXXLQDIMSR

SEQ ID NO: 2 - protein, 30 aa, Artificial Sequence
  Chemically Synthesized
YXDAIFTXXXRKVLXQLSARXXLQDIMSRX

SEQ ID NO: 3 - protein, 31 aa, Artificial Sequence
  Chemically Synthesized
YXDAIFTXXXRKVLXQLSARXXLQDIMSRXX

SEQ ID NO: 4 - protein, 32 aa, Artificial Sequence
  Chemically Synthesized
YXDAIFTXXXRKVLXQLSARXXLQDIMSRXXX

SEQ ID NO: 5 - protein, 33 aa, Artificial Sequence
  Chemically Synthesized
YXDAIFTXXXRKVLXQLSARXXLQDIMSRXXXX

SEQ ID NO: 6 - protein, 34 aa, Artificial Sequence
  Chemically Synthesized
YXDAIFTXXXRKVLXQLSARXXLQDIMSRXXXXX

SEQ ID NO: 7 - protein, 35 aa, Artificial Sequence
  Chemically Synthesized
YXDAIFTXXXRKVLXQLSARXXLQDIMSRXXXXXX

SEQ ID NO: 8 - protein, 36 aa, Artificial Sequence
  Chemically Synthesized
YXDAIFTXXXRKVLXQLSARXXLQDIMSRXXXXXXX

SEQ ID NO: 9 - protein, 37 aa, Artificial Sequence
  Chemically Synthesized
YXDAIFTXXXRKVLXQLSARXXLQDIMSRXXXXXXXX

SEQ ID NO: 10 - protein, 38 aa, Artificial Sequence
  Chemically Synthesized
YXDAIFTXXXRKVLXQLSARXXLQDIMSRXXXXXXXXX

SEQ ID NO: 11 - protein, 39 aa, Artificial Sequence
  Chemically Synthesized
YXDAIFTXXXRKVLXQLSARXXLQDIMSRXXXXXXXXXX

SEQ ID NO: 12 - protein, 40 aa, Artificial Sequence
  Chemically Synthesized
YXDAIFTXXXRKVLXQLSARXXLQDIMSRXXXXXXXXXXX

SEQ ID NO: 13 - protein, 41 aa, Artificial Sequence
  Chemically Synthesized
YXDAIFTXXXRKVLXQLSARXXLQDIMSRXXXXXXXXXXXX

SEQ ID NO: 14 - protein, 42 aa, Artificial Sequence
  Chemically Synthesized
YXDAIFTXXXRKVLXQLSARXXLQDIMSRXXXXXXXXXXXXX

SEQ ID NO: 15 - protein, 43 aa, Artificial Sequence
  Chemcially Synthesized
YXDAIFTXXXRKVLXQLSARXXLQDIMSRXXXXXXXXXXXXXX

SEQ ID NO: 16 - protein, 44 aa, Artificial Sequence
  Chemically synthesized
YXDAIFTXXXRKVLXQLSARXXLQDIMSRXXXXXXXXXXXXXXX

(19) United States
(12) Patent Application Publication
(10) Pub. No.: US 2009/0088380 A1
(43) Pub. Date: Apr. 2, 2009

(54) GHRH ANALOGS AND THERAPEUTIC USES THEREOF

(76) Inventors: Pierrette Gaudreau, Brossard (CA); Hanna Sikorska, Montreal (CA)

Correspondence Address: MEYERTONS, HOOD, KIVLIN, KOWERT & GOETZEL, P.C., P.O. BOX 398, AUSTIN, TX, 78767-0398, US

(21) Appl. No.: 12/171,447

(22) Filed: Jul. 11, 2008

Related U.S. Application Data
(60) Provisional application No. 60/949,442, filed on Jul. 12, 2007.

(51) Int. Cl. A61K 38/18; A61P 3/00; A61P 1/00

(52) U.S. Cl. 514/12

(57) ABSTRACT

Described herein are growth hormone-releasing hormone (GHRH) analogs and uses for such analogs. In some embodiments, growth hormone related diseases may be treated with one or more synthetic GHRH analogs of 29 amino acids or more, exhibiting concomitantly an increased resistance to proteolysis and high binding affinity to human GHRH receptor in in vitro studies, in comparison with human native GHRH (1-29)NH₂.

### PRIORITY CLAIM

This application claims priority to U.S. Provisional Patent Application Ser. No. 60/949,442, which is entitled “GHRH ANALOGS AND THERAPEUTIC USES THEREOF” and which is incorporated herein by reference.

### BACKGROUND OF THE INVENTION

1. Field of the Invention

The present invention generally relates to the field of growth hormone-releasing hormone (GHRH) analogs. More particularly, the invention relates to GHRH analogs of at least 29 amino acids, which exhibit increased resistance to proteolysis and bind to the human GHRH receptor (hGHRH-R) with higher affinity in vitro than native human GHRH (1-29)NH₂.

2. Description of the Relevant Art

Growth hormone (GH) is a somatotropic anterior pituitary hormone responsible for regulating growth and exerting anabolic functions, such as stimulating protein synthesis and accretion, and lipolysis. Until the mid 1980's, the only source of human GH (hGH) was from pituitary glands collected post mortem. Today, hGH is available in large quantities through genetic engineering.

GH promotes growth in children and plays an important role in adult metabolism. GH deficiencies in children are associated with growth retardation or failure while GH excess causes gigantism or acromegaly, respectively.

GH is produced in somatotroph cells of the anterior pituitary gland of mammals and secreted throughout life. It is mainly controlled in the brain by two hypothalamic peptides: GHRH, which stimulates its secretion and synthesis; and somatostatin, which inhibits them. A number of peripheral factors regulate GH secretion. Among them, insulin-like growth factor-1 (IGF-1) represents an important one as it is produced by the liver in response to GH and acts on the hypothalamus to exert a negative feedback on GH secretion.

Pharmaceutical agents that target the GH axis include synthetic GHRH that stimulates GH release; a somatostatin analog, octreotide, that inhibits GH release; recombinant human GH (somatotropin, somatrem) that is used to replace GH in a state of deficiency; and recombinant IGF-1 that is used to treat GH insensitivity (Laron-type dwarfism).

GH declines with age in every animal species that have been tested to date. In humans, the amount of GH after the age of 21 to 31 falls by about 14% per decade, so that the total 24-hour GH production rate is reduced in half by the age of 60. Humans thus daily produce GH at about 500 μg at 20 years of age, 200 μg at 40 years, and 25 μg at 80 years old.

With the availability of biosynthetic GH for prescription use in the US since 1985, GH replacement therapy has been the treatment of choice in cases of growth hormone deficiency. In the US, the number of children eligible for GH treatment ranges from 11,000, if strict criteria for GH deficiency are applied, to 1.3 million, if all those with heights below the third percentile are candidates. The respective cost of GH therapy would jump from $155 million to $20 billion per year if the less stringent criterion became the standard of care. So far, pediatricians in the US have shown gratifying restraint in prescribing GH for non-approved indications, since only 20,000 children are receiving GH therapy.

Another problem is the low patient compliance, as conventional biosynthetic GH has to be injected. The complex amino acid structure of GH (191 amino acids) is completely destroyed in the gastrointestinal tract.

Overall, GH is contraindicated in patients with active malignant disease, benign intracranial hypertension, and proliferative or preproliferative diabetic retinopathy.

Growth hormone releasing hormone (GHRH) is a peptide of 44 amino acids. Several authors have reported that GHRH(1-29) NH₂, the 29 amino acid N-terminus fragment of GHRH(1-44) NH₂, exhibits the full bioactivity of GHRH(1-44) NH₂.

GHRH was first isolated from pancreatic tumours and subsequently from the hypothalamus of various mammals. In addition to the arcuate nucleus of the hypothalamus, GHRH is present in other hypothalamic nuclei such as the suprachiasmatic nucleus and in the other regions of the brain such as the limbic system. GHRH-like immunoreactivity and/or GHRH messenger ribonucleic acid (mRNA) has also been found in the placenta, gastrointestinal tract, ovary, testis, thymus, spleen and renal medulla.

GHRH binding sites have been localized and characterized in various tissue preparations and cell cultures from normal and tumoral pituitary, and from normal hypothalamus, testis, ovary and renal medulla. Pharmacological studies have demonstrated the existence of two populations of GHRH binding sites in the pituitary and ovary: a high affinity and low capacity binding site, corresponding to the physiologically relevant form of the receptor, and a low affinity and high capacity binding site. Alterations of the rat pituitary GHRH binding site parameters occur in the course of aging, leading to a loss of the high affinity binding sites.

GHRH is known to degrade rapidly in vivo. Degradation patterns of GHRH have been elucidated in serum and plasma, liver and target tissues such as the pituitary gland and hypothalamus. The vulnerable peptides identified so far are R2-R3, R10-R11, R11-R12, R14-R15, R18-R19, R20-R21, R21-R22. Furthermore, it is also known that modifications at these amino acid residues can prevent or decrease proteolysis as well as result in a longer duration of action of GHRH and its analogs.

These caveats and limitations in naturally occurring GHRH resulted in the discovery of a new class of fourteen (14) polysubstituted synthetic GHRH superagonists, exhibiting a 5 to 13-fold increase in affinity to rat pituitary GHRH receptor, as described in U.S. Pat. No. 5,854,216, which is incorporated herein by reference. Such an invention provided non-toxic highly sensitive and selective marker peptides and marker polyclonal antibodies of the GHRH receptors.

In addition, GHRH analogs designed so far, either from academic organizations or pharmaceutical/biotechnology companies, were based on structural changes of these analogs aimed at merely improving their half-life in bioassays or in vivo experiments on animals.

### SUMMARY OF THE INVENTION

The embodiments described herein provide therapeutically useful GHRH analogs and/or pharmaceutically acceptable salts thereof, which are suitable for use in treating or preventing disorders or diseases associated with GHRH and/or GH function, such disorders or diseases including but not limited to protein catabolism in acute illnesses such as burns, infection (sepsis), chronic or acute renal failure, cardiac failure, protein catabolism in chronic diseases such as COPD, osteoporosis, cancer-related cachexia, post-surgical complications, wound healing, lactation failure, infertility in women, muscle wasting diseases, cancer, metabolic syndrome, protein malnutrition following long-term corticosteroid therapy, GI malabsorption (SBS, Crohn's disease), radiotherapy, chemotherapy-related side effects, short statue, hypothalamic pituitary dwarfism, T-cell and B-cell immunodeficiencies, neurodegenerative conditions, aging, sleep disorders, lack of appetite, lipodystrophy, non-union bone-fracture, acute/chronic debilitating illness or infection, anabolic and/or catabolic problems, and GHRH receptor-dependent tumors. As used herein, muscle-wasting diseases could be any one of the following: sarcopenia, frailty in the elderly, age-related sarcopenia, muscular dystrophy, HIV and cancer, chronic renal failure, kidney disease, amyotrophic lateral sclerosis (ALS or Lou Gehrig's disease), burns, diabetic neuropathy, Guillain-Barre syndrome, long-term corticosteroid therapy, long-term immobilization, osteoarthritis, rheumatoid arthritis, peripheral neuropathy, polio, spinal cord injury, Chronic obstructive pulmonary disease (“COPD”), and stroke.

Accordingly, the presently described embodiments are directed to GHRH analogs and pharmaceutically acceptable salts thereof and to pharmaceutical compositions containing such analogs. The presently described embodiments are yet further directed to therapeutic uses of such compositions, and to methods for initiating GHRH-induced biological processes.

In a first non-limiting set of embodiments, a GHRH analog, a derivative thereof, or a pharmaceutically acceptable salt thereof may have Formula X:

Tyr-A2-Asp-Ala-Ile-Phe-Thr-A8-A9-A10-Arg-Lys-Val-
Leu-A15-Gln-Leu-Ser-Ala-Arg-A21-A22-Leu-Gln-Asp-
Ile-Met-Ser-Arg-A30-NH₂

where A2 is Ala or D-Ala; A8 is Asn, D-Asn or Ala; A9 is Ser or Ala; A10 is Tyr or D-Tyr; A15 is Gly, Ala or D-Ala; A21 is Lys or D-Lys; A22 is Leu, D-Leu, Lys or Ala; and A30 is a bond or any amino acid sequence of 1 up to 15 residues;

Where said analog, derivative of said analog or salt thereof having an in vitro potency index substantially higher than the in vitro potency index of a naturally occurring GHRH.

In a second non-limiting set of embodiments, a pharmaceutical composition may include the above-mentioned analog, derivative or salt thereof, in combination with one or more pharmaceutically acceptable carriers, and/or stabilizers and/or bioavailability enhancers.

In a further non-limiting set of embodiments, a use of the analog, derivative, salt or pharmaceutical composition set forth above may include the administration thereof to a subject for the purpose of evoking the in vivo release of GH and IGF-1 in said subject.

In yet a further non-limiting set of embodiments, a use of the analog, derivative, salt or pharmaceutical composition set forth above may include the use thereof for the preparation of a drug that is suitable for use in treating or preventing disorders or diseases associated with GHRH and/or GH and/or IGF-1 function, or the function of their corresponding tissue receptors or plasma binding factors inhibiting the function of GHRH, GH and IGF (e.g., somatomedines).

In yet another non-limiting set of embodiments, a method for initiating GHRH-induced biological processes may include contacting the analog, derivative, salt or pharmaceutical composition set forth above with a cell, a group of cells, or a tissue that expresses GHRH-R.

The invention and its advantages will be better understood upon reading the following non-restricted description of preferred embodiments thereof, made with references to the accompanying drawings.

### BRIEF DESCRIPTION OF THE DRAWINGS

Other objects and advantages of the invention will become apparent upon reading the following detailed description and upon reference to the accompanying drawings in which:

FIG. 1 shows a graphic representation of the secretion profile of rat growth hormone following a single intravenous injection of a GHRH analog, at escalating doses, versus natural human GRF(1-44)NH₂ peptide;

FIG. 2 shows a graphic representation of the secretion profile of rat growth hormone following a single subcutaneous injection of a GHRH analog, at escalating doses;

FIG. 3 shows a graphic representation of the secretion profile of canine growth hormone following multiple subcutaneous injections of a GHRH analog, at escalating doses;

FIG. 4 is a flow chart showing the progression of human clinical trials on days 0 and 1;

FIG. 5 is a flow chart showing the progression of human clinical trials on days 28 and 29; and

FIG. 6 depicts metabolic pathways for deranged GH and IGF-I axis in chronic kidney disease.

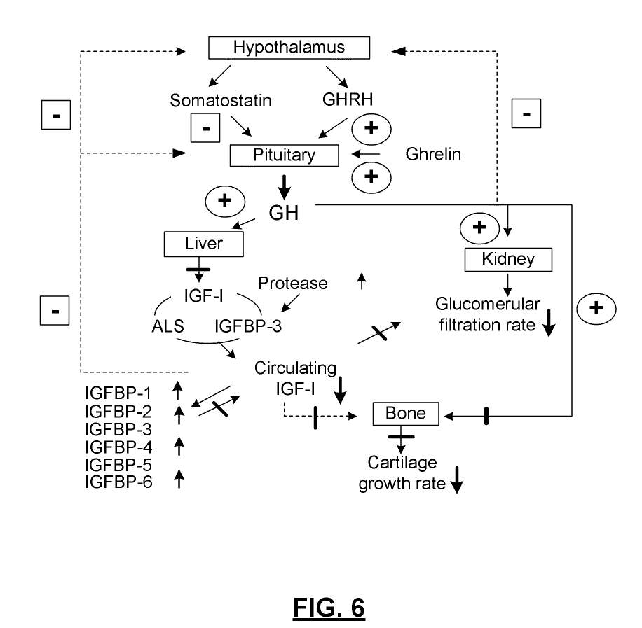

While the invention is susceptible to various modifications and alternative forms, specific embodiments thereof are shown by way of example in the drawings and will herein be described in detail. It should be understood, however, that the drawing and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE EMBODIMENTS

Embodiments described herein are directed to GHRH analogs that exhibit increased resistance to proteolysis and have a relatively high binding affinity to human GHRH receptor in in vitro studies, in comparison with human native GHRH (1-29)NH₂. As used herein the term “GHRH analog” means a GHRH agonist, more specifically a synthetic peptide that binds with high affinity to the GHRH receptor and increases plasma growth hormone (GH) concentration by stimulating somatotroph cells of the anterior pituitary gland to release GH, and in turn, to secrete and release IGF-1.

Embodiments described herein further include compositions that comprise a GHRH analog as defined herein and methods of use of such GHRH analogs and/or compositions.

### DEFINITIONS

It is to be understood that the present invention is not limited to particular devices or biological systems, which may, of course, vary. It is also to be understood that, as used in this specification and the appended claims, the singular forms “a”, “an”, and “the” include singular and plural referents unless the content clearly dictates otherwise. Thus, for example, reference to “a linker” includes one or more linkers. It is to be yet further understood that any terminology used herein is for the purpose of describing particular embodiments only, and is not intended to be limiting.

The terms used throughout this specification generally have their ordinary meanings in the art, within the context of the invention, and in the specific context where each term is used. Certain terms are discussed below, or elsewhere in the specification, to provide additional guidance to the practitioner in describing the general embodiments of the invention, as well as how to make and use them. It will be readily appreciated that the same thing can be said in more than one way. Consequently, alternative language and synonyms may be used for any one or more of the terms discussed herein, nor is any special significance to be placed upon whether or not a term is elaborated or discussed in greater detail herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term.

As used herein, the term “substantially identical”, when used in reference to a polynucleotide, generally refers to a polynucleotide, or a portion or fragment thereof, whose nucleotide sequence is at least 95%, 90%, 85% 80%, 70%, 60% or 50% identical to the nucleotide sequence of a reference polynucleotide. When used in reference to a polypeptide, the term generally refers to a polypeptide, or a fragment thereof, whose amino acid sequence is at least 95%, 90%, 85% 80%, 70%, 60% or 50% identical to the amino acid sequence of a reference polypeptide. For polypeptides, the length of comparison sequences will generally be at least about 5 amino acids, and may include the complete polypeptide sequence. For nucleic acids, the length of comparison sequences will generally be at least about 15 nucleotides, and may include the complete reference nucleic acid sequence. Sequence identity between two or more polypeptide or nucleic acid sequences is typically determined using sequence analysis software (e.g., Sequence Analysis Software Package of the Genetics Computer Group, University of Wisconsin Biotechnology Center) designed for this purpose. Such software matches similar sequences by assigning degrees of homology to various substitutions, deletions, substitutions, and other modifications. Conservative substitutions typically include substitutions within the following groups:

Gly;
Ala;
Val, Ile, Leu;
Asp, Glu, Asn, Gln;
Ser, Thr;
Lys, Arg;
and
Phe, Tyr.

As used herein, the term “recombinant,” when used in reference to a polynucleotide or a protein, generally refers to a polynucleotide or a polypeptide molecule that is produced using genetic engineering techniques and that is distinct from a naturally occurring nucleic acid or polypeptide molecule. Recombinant DNA (sometimes represented as “rDNA”) is an artificial DNA sequence resulting from the combining of two other DNA sequences in a plasmid/vector. The term recombinant DNA refers to a new combination of DNA molecules that are not found together naturally. Although processes such as crossing over (genetic recombination) technically produce recombinant DNA, the term is generally reserved for DNA produced by joining molecules derived from different biological sources.

The term “portion”, as used herein, in the context of a molecule, such as a polypeptide or of a polynucleotide (as in “a portion of a given polypeptide/polynucleotide”) generally refers to fragments of that molecule. The fragments may range in size from three amino acid or nucleotide residues to the entire molecule minus one amino acid or nucleotide. Thus, for example, a polypeptide “comprising at least a portion of the polypeptide sequence” encompasses the polypeptide defined by the sequence, and fragments thereof, including but not limited to the entire polypeptide minus one amino acid.

As used herein, the term “amino acid” generally refers to naturally occurring or synthetic amino acids, as well as amino acid analogs and amino acid mimetics that function in a manner similar to naturally occurring amino acids. Naturally occurring amino acids are those encoded by the genetic code, as well as those amino acids that are later modified, e.g., hydroxyproline, carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group. Examples of amino acid analogs include, but are not limited to, homoserine, norleucine, methionine sulfoxide, methionine, and methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

When used herein in the context of polypeptides/polynucleotides, the term “analogs,” (which may be used synonymously with the term “variant”) generally refers to two or more structurally similar polypeptides/polynucleotides that are characterized by differences in amino acid/nucleotide sequence (e.g., having at least 10%, at least 20%, at least 30%, at least 50%, at least 75%, at least 85%, or at least 95% sequence identity) and/or in biochemical modifications (e.g., post-translational modification and the like). While a subset of the general activities of certain analogs may be similar, structural differences occurring between the analogs may result in at least a portion of their activities being non-overlapping. An “analog” may refer to a polynucleotide or a polypeptide molecule that is altered at one or more regions, including alterations in the nucleotide or amino acid sequence, as well as covalent modifications of the molecule, relative to the polynucleotide or a polypeptide molecule as it is found in nature. Thus, in some instances, the terms “analog,” “variant” and “isoform” may be used interchangeably. Illustrative examples of such analogs would include, by way of example only, polypeptides in which replacement of a hydrogen group by an alkyl, acyl, thiol, amide or other such functional group has occurred at one or more amino acid residues. An analog may have “conservative” changes, wherein a substituted amino acid may have similar structural and/or chemical properties (e.g., replacement of a non-polar amino acid residue with a different non-polar amino acid residue). A variant may also have “nonconservative” changes (e.g., replacement of a polar amino acid residue with a non-polar or a charged amino acid residue). Variants may also include similar minor variations in amino acid sequence including, but not limited to, deletions, truncation, insertions, or combinations thereof. Guidance in determining which amino acid residues may be substituted, inserted, or deleted without abolishing or otherwise substantially affecting biological activity is widely available in the art. Further guidance may be found using computer programs well known in the art, for example, DNASTAR software. In general, a GHRH analog will retain at least a subset of the biological functions typically associated with native GHRH, such as, for example, the ability to bind to a GHRH-R, and/or the ability to stimulate GH and/or IGF-1 secretion in certain cells, or the ability to affect various other physiological parameters associated with native GHRH. The term “analog” may also refer to a polypeptide or polynucleotide whose polypeptide or polynucleotide sequence is altered at one or more positions and is different from what normally appears, occurs, or functions in nature. In the context of at least some of the present embodiments, the term refers to a polypeptide or polynucleotide that differs in sequence from the native polypeptide or polynucleotide at one or more positions. Mutations may include deletions, truncations, insertions, substitutions, or combinations thereof, of one or more amino acids or nucleotides in a polypeptide or polynucleotide, respectively.

A “deletion”, as used herein, generally refers to a change in either amino acid or nucleotide sequence in which one or more amino acid or nucleotide residues are absent. A deletion may occur at any position along a polypeptide or polynucleotide molecule.

An “insertion” or “addition,” as used herein, generally refers to a change in an amino acid or nucleotide sequence resulting in the addition of one or more amino acid or nucleotide residues, respectively, as compared to the naturally occurring molecule. An insertion may occur at any position along a polypeptide or polynucleotide molecule.

A “substitution,” as used herein, refers to the replacement of one or more amino acids or nucleotides by different amino acids or nucleotides, respectively.

A “truncation”, as used herein, refers to the removal (i.e. deletion) of one or more amino acids or nucleotides from amino- or carboxy-terminal, or from the 5′- or 3′-end, of a polypeptide or polynucleotide, respectively.

As used herein, the term “recombinant,” when used in reference to a polynucleotide or a polypeptide, generally refers to a polynucleotide or a polypeptide molecule that is produced using genetic engineering techniques and that it is distinct from a naturally occurring nucleic acid or polypeptide molecule.

The term “portion”, as used herein, in the context of a molecule, such as a polypeptide or of a polynucleotide (as in “a portion of a given polypeptide/polynucleotide”) generally refers to fragments of that molecule. The fragments may range in size from three amino acid or nucleotide residues to the entire molecule minus one amino acid or nucleotide. Thus, for example, a polypeptide “comprising at least a portion of the polypeptide sequence” encompasses the polypeptide defined by the sequence, and fragments thereof, including but not limited to the entire polypeptide minus one amino acid.

The term “wild-type” is used herein to indicate a polypeptide or a polynucleotide that contains only those amino acid or nucleotide sequences found in the protein or nucleic acid molecule as it typically occurs in nature. In other words, a wild-type molecule is a molecule that is substantially free of natural, spontaneous or experimentally induced mutations. A wild-type polypeptide or polynucleotide may be produced by recombinant means or may be isolated from a naturally occurring source.

As used herein, the term “endogenous,” generally refers to a factor, such as a gene or a polypeptide, that originates from a naturally occurring source within a cell or organism. An “endogenous gene” generally refers to a gene that is a part of the original genetic repertoire of a cell or an organism. An endogenous gene may be chromosomal or extra-chromosomal (e.g. mitochondrial genes). An endogenous gene may be wild type or mutant. An “endogenous protein” generally refers to a protein that is produced from an endogenous gene.

As used herein, the term “exogenous” generally refers to a factor that originates from a source that is outside of a cell or an organism. An “exogenous gene” generally refers to a gene that is not a part of the original genetic repertoire of a cell or an organism. An exogenous gene may be delivered to a cell or a group of cells using one or more gene delivery or transfection systems. An exogenous gene may be recombinant (e.g., a gene that has been inserted into a vector), or may be naturally occurring (e.g., a gene that is part of the naturally occurring genome of a virus). An exogenous gene may be chromosomal (e.g., as a stably integrated “transgene”) or extra-chromosomal (e.g., as an unintegrated vector).

As used herein, “conserved region” or “similar” or “related,” (when used in the context of comparing the sequence of two or more reference peptides) generally refers to any stretch of six or more contiguous amino acids in a polypeptide that exhibit at least 30%, or between 50% to 70%, or between 60% to 95% amino acid sequence identity to the corresponding region of one or more reference polypeptides.

As used herein, the term “pharmaceutical composition” or “pharmaceutical preparation” generally refers to a formulation that has been adapted to deliver a prescribed dosage of one or more therapeutically useful agents to a cell, a group of cells, an organ or tissue, an animal or a human. A pharmaceutical preparation may be prepared as a solid, semi-solid, gel, hydrogel, liquid, solution, suspension, emulsion, aerosol, powder, or combinations thereof. Included in a pharmaceutical preparation may be one or more carriers, preservatives, flavorings, excipients, coatings, stabilizers, binders, solvents and/or auxiliaries.

As used herein, the term “tissue”, when used in reference to a part of a body or of an organ, generally refers to an aggregation or collection of morphologically similar cells and associated accessory and support cells and intercellular matter, including extracellular matrix material, vascular supply, and fluids, acting together to perform specific functions in the body. There are generally four basic types of tissue in animals and humans including muscle, nerve, epithelial, and connective tissues.

As used herein, terms such as “biological availability,” “bioavailablity,” or the like generally refer to the relative amount of a biologically active factor or substance that is available to carry out a biological function.

As used herein, the term “polypeptide” generally refers to a naturally occurring, recombinant or synthetic polymer of amino acids, regardless of length or post-translational modification (e.g., cleavage, phosphorylation, glycosylation, acetylation, methylation, isomerization, reduction, farnesylation, etc. . . . ), that are covalently coupled to each other by sequential peptide bonds. Although a “large” polypeptide is typically referred to in the art as a “protein” the terms “polypeptide” and “protein” are often used interchangeably. In general, the first amino acid residue or group of amino acid residues in a polypeptide are said to be at the “amino-terminal” or “N-terminal” of the polypeptide. Similarly, the last amino acid residue, or group of amino acid residues in a polypeptide are said to be at the “carboxy-terminal” or “C-terminal”.

The term “treating” as used herein refers to administering a pharmacologically active composition prior to, during, or after the onset of clinical symptoms. The terms “in need of treatment,” “in need thereof” or “who would benefit from such treatment” as used herein refers to a judgment made by a caregiver that an individual or animal requires or will benefit from treatment. This judgment is made based on a variety of factors that are in the realm of a caregiver's expertise, but includes the knowledge that the individual or animal is ill, or will be ill, as the result of a condition that is treatable by the methods embodied herein.

The term “disorders or diseases associated with GHRH and/or GH function,” generally refer to any pathological situation in which reduced GHRH function and/or reduced GH function are implicated as contributing the etiology thereof. Non-limiting examples of such disorders or diseases include, though are not limited to: protein catabolism in acute illnesses such as burns, infection (sepsis), chronic or acute renal failure, cardiac failure, protein catabolism, osteoporosis, cancer-related cachexia, post-surgical complications, wound healing, lactation failure, infertility in women, muscle wasting diseases, cancer, metabolic syndrome, protein malnutrition following long-term corticosteroid therapy, GI malabsorption (SBS, Crohn's disease), radiotherapy, chemotherapy-related side effects, short statue, hypothalamic pituitary dwarfism, T-cell and B-cell immunodeficiencies, neurodegenerative conditions, aging, sleep disorders, lack of appetite, lipodystrophy, non-union bone-fracture, acute/chronic debilitating illness or infection, anabolic and/or catabolic problems, and GHRH receptor-dependent tumors. As used herein, muscle-wasting diseases could be any one of the following: sarcopenia, frailty in the elderly, age-related sarcopenia, muscular dystrophy, HIV and cancer, chronic renal failure, kidney disease, amyotrophic lateral sclerosis (ALS or Lou Gehrig's disease), burns, diabetic neuropathy, Guillain-Barre syndrome, long-term corticosteroid therapy, long-term immobilization, osteoarthritis, rheumatoid arthritis, peripheral neuropathy, polio, spinal cord injury, Chronic obstructive pulmonary disease (“COPD”), and stroke.

As used herein, the term “muscle-wasting disease” generally refers to a group of disorders in which sarcopenia is a major consequence. Exemplary though non-limiting muscle-wasting diseases include any one of the following: sarcopenia, frailty in the elderly, age-related sarcopenia, muscular dystrophy, HIV, cancer, chronic renal failure, kidney disease, amyotrophic lateral sclerosis (ALS or Lou Gehrig's disease), burns, diabetic neuropathy, Guillain-Barre syndrome, long-term corticosteroid therapy, long-term immobilization, osteoarthritis, rheumatoid arthritis, peripheral neuropathy, polio, spinal cord injury, and stroke.

### GHRH Analog, Derivative or Salt Thereof

In one embodiment, the GHRH analog peptides may be synthesized using chemical synthetic techniques. In another embodiment, GHRH analog peptides may be made using recombinant techniques. The present invention also relates to derivatives, fragments, homologs, variants and salts of the aforementioned peptides.

According to the first aspect, the present invention relates to a GHRH analog, a functional derivative or a pharmaceutically acceptable salt thereof. More specifically, the GHRH analog of the invention has an amino acid sequence comprising the following Formula X:

Tyr-A2-Asp-Ala-Ile-Phe-Thr-A8-A9-A10-Arg-Lys-Val-
Leu-A15-Gln-Leu-Ser-Ala-Arg-A21-A22-Leu-Gln-Asp-
Ile-Met-Ser-Arg-A30-NH₂

where A2 is Ala or D-Ala; A8 is Asn, D-Asn or Ala; A9 is Ser or Ala; A10 is Tyr or D-Tyr; A15 is Gly, Ala or D-Ala; A21 is Lys or D-Lys; and A22 is Leu, D-Leu, Lys or Ala, and A30 is a bond or any amino acid sequence of 1 up to 15 residues. In some embodiments, GHRH analog peptides may be amidated at the C-terminal thereof.

The term “residue”, when used with reference to an amino acid, means a radical derived from the corresponding amino acid by eliminating the hydroxyl of the carboxyl group a hydrogen of the amino group.

Furthermore, the GHRH analog of the invention has an in vitro potency index substantially higher than the in vitro potency index of a naturally occurring GHRH. It will be understood that the expression “naturally occurring GHRH” encompasses both hGHRH (1-29)NH₂ (the functional portion of the native GHRH peptide) and hGHRH (1-44)NH₂ (the complete native GHRH peptide).

As used herein, the expression “in vitro potency index” represents a tool of comparison which results from multiplying: 1-the relative binding affinity of GHRH analogs compared with the native hGHRH (1-29)NH₂, in BHK cells expressing the hGHRH receptor; with 2-the relative resistance to in vitro proteolysis of compounds in comparison with hGHRH (1-29)NH₂ after 60 or 180 minute-incubations in human plasma or human serum.

As used herein, the term “a relatively high binding affinity” means that the GHRH analog has a binding affinity to human GHRH receptor of at least about 100-fold higher than the binding affinity of the native GHRH.

As used herein, the term “increased resistance to proteolysis” means that the GHRH analog of the invention, upon in vitro incubation in human plasma or serum, has a substantially higher mean residual amount percentage, such as at least about 50%, in comparison with the native GHRH.

The expression “substantially higher”, used to characterize the in vitro potency index of the present GHRH analog, derivative or salt thereof, indicates an in vitro potency index preferably at least 500-fold higher, more preferably 1500-fold higher and even more preferably 2500-fold higher than the in vitro potency index of the native hGHRH (1-29)NH₂.

As used herein the term “functional derivative”, as is generally understood, refers to a protein/peptide sequence that possesses a functional biological activity that is substantially similar to the biological activity of the GHRH analog of the present invention. A functional derivative of a GHRH analog may or may not contain post-translational modifications, such as a covalently linked carbohydrate, if such modification is not necessary for the performance of a specific function. The term “functional derivative” encompasses “fragments”, “segments”, “variants”, or “chemical derivatives” of a GHRH analog.

In general, the abbreviations used herein for designating the amino acids are based on recommendations of the IUPAC-IUB Commission on Biochemical Nomenclature (Biochemistry, 1972, 11: 1726-1732). More specifically, the term “amino acid” is includes alanine, arginine, asparagine, aspartic acid, cysteine, glutamic acid, glutamine, glycine, histidine, hydroxylysine, hydroxyproline, isoleucine, leucine, lysine, methionine, phenylalanine, proline, pyroglutamic acid, sarcosine, serine, threonine, tryptophan, tyrosine and valine.

The GHRH peptides described herein may be synthesized by using solid-phase peptide chemistry (e.g., with a t-Boc-Acid-Labile protection scheme). Nevertheless, it will be readily understood by the skilled practitioner that the subject GHRH analogs of the invention may be prepared using any number of conventional techniques known to one skilled in the art, including chemical or recombinant means, without departing from the spirit and scope of the present disclosure. Further non-limiting examples of synthetic methods that are readily suited to producing the subject peptides are set forth in greater detail below.

According to one embodiment, different combinations of polysubstitutions in the native form of GHRH may be used. Accordingly, in one embodiment, a GHRH analog has the above-mentioned Formula X with the following substitutions: A2 is D-Ala, A8 is Asn, A9 is Ser; A10 is D-Tyr; A15 is D-Ala, A21 is Lys; A22 is Lys and A30 is a bond. In the experimental procedures described herein this particular analog is referred to as the GHRH(1-29)NH₂ synthetic analog.

Another GHRH analog has the Formula X wherein A2 is D-Ala, A8 is Ala, A9 is Ser, A10 is Tyr, A15 is Ala, A21 is Lys, A22 is Lys, and A30 is a bond.

Another GHRH analog has the Formula X wherein A2 is Ala, A8 is Ala, A9 is Ala, A10 is Tyr, A15 is Ala, A21 is Lys, A22 is Ala and A30 is a bond.

Another GHRH analog has the Formula X wherein A2 is D-Ala, A8 is Asn, A9 is Ser, A10 is D-Tyr, A15 is Gly, A21 is Lys, A22 is Lys and A30 is a bond.

Another GHRH analog has the Formula X wherein A2 is D-Ala, A8 is Ala, A9 is Ser, A10 is D-Tyr, A15 is Ala, A21 is D-Lys, A22 is Lys and A30 is a bond.

In one embodiment, GHRH peptide analogs are related to mammalian GHRH. In one another embodiment, the peptides are related to a human GHRH.

Homologs of the disclosed peptides are also provided. “Variant” refers to a polynucleotide or polypeptide differing from the related polynucleotide or polypeptide, but retaining essential properties thereof. Generally, variants are overall closely similar, and in many regions, identical to the related polynucleotide or polypeptide. The variants may contain alterations in the coding regions, non-coding regions, or both.

### Isolated Peptides and Polynucleotides

GenBank Accession numbers for mouse, human and rat GHRH peptides, providing the amino acid sequences that form the basis of the GHRH analogs of the present disclosure are NP_{—}034415, NP_{—}066567 and NP_{—}113765, respectively. GeneBank accession numbers for the mouse and human transcripts of GHRH are NM_{—}010285 and NM_{—}001009824 (transcript 2 variant) and NM_{—}000822 (transcript 1 variant). The actual sequence of each peptide disclosed in the instant invention can readily be determined by comparison therein. The predicted amino acid sequence can then be determined from its nucleotide sequence using standard protocols well known in the art. The amino acid sequence of the peptide encoded by a particular clone can also be determined by expression of the clone in a suitable host cell, collecting the peptide and determining its sequence.

Derivatives, fragments, and analogs provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively. Fragments are, at most, one nucleic acid-less or one amino acid-less than the wild type full-length sequence. Derivatives and analogs may be full length or other than full length, if said derivative or analog contains a modified nucleic acid or amino acid, as described infra. Derivatives or analogs of the aforementioned peptides include, but are not limited to, molecules comprising regions that are substantially homologous to the aforementioned peptides, in various embodiments, by at least about 30%, 50%, 70%, 80%, or 95% identity (with a preferred identity of 80-95%) over an amino acid sequence of identical size or when compared to an aligned sequence in which the alignment is done by computer homology programs known in the art, or whose encoding nucleic acid is capable of hybridizing to the complement (e.g., the inverse complement) of a sequence encoding the aforementioned peptides under stringent, moderately stringent, or low stringent conditions.

The aforementioned peptides are, in some embodiments, functionally active. In some embodiments, the aforementioned peptides, and fragments, derivatives, homologs or analogs thereof, are related to animals (e.g., mouse, rat, pig, cow, dog, monkey, frog), insects (e.g., fly), plants or, most preferably, human GHRH. As utilized herein, the term “functionally active” refers to species displaying one or more known functional attributes of a full-length GHRH.

Recombinant Technologies for Obtaining the Aforementioned Peptides

The aforementioned peptides may be obtained by methods well-known in the art for peptide purification and recombinant peptide expression. For recombinant expression of one or more of the peptides, the nucleic acid containing all or a portion of the nucleotide sequence encoding the peptide may be inserted into an appropriate expression vector (i.e., a vector that contains the necessary elements for the transcription and translation of the inserted peptide coding sequence). In a preferred embodiment, the regulatory elements are heterologous (i.e., not the native gene promoter). Alternately, the necessary transcriptional and translational signals may also be supplied by the native promoter for the genes and/or their flanking regions.

Host-Vector Systems

A variety of host-vector systems may be utilized to express the peptide coding sequence(s). These include, but are not limited to: (i) mammalian cell systems that are infected with vaccinia virus, adenovirus, and the like; (ii) insect cell systems infected with baculovirus and the like; (iii) yeast containing yeast vectors or (iv) bacteria transformed with bacteriophage, DNA, plasmid DNA, or cosmid DNA. Depending upon the host-vector system utilized, any one of a number of suitable transcription and translation elements may be used.

Any of the methodologies known within the relevant prior art regarding the insertion of nucleic acid fragments into a vector may be utilized to construct expression vectors that contain a chimeric gene comprised of the appropriate transcriptional/translational control signals and peptide-coding sequences. Promoter/enhancer sequences within expression vectors may utilize plant, animal, insect, or fungus regulatory sequences.

Promoter/enhancer elements from yeast and other fungi (e.g., the Gal4 promoter, the alcohol dehydrogenase promoter, the phosphoglycerol kinase promoter, the alkaline phosphatase promoter), as well as from animal transcriptional control regions, for example, those that possess tissue specificity and have been used in transgenic animals, may be utilized in the production of GHRH peptides and analogs described herein. Transcriptional control sequences derived from animals include, but are not limited to: (i) the insulin gene control region active within pancreatic .beta.-cells; (ii) the immunoglobulin gene control region active within lymphoid cells; (iii) the albumin gene control region active within liver; (iv) the myelin basic protein gene control region active within brain oligodendrocyte cells; and (v) the gonadotrophin-releasing hormone gene control region active within the hypothalamus, and the like. In an embodiment, a vector is utilized that is comprised of a promoter operably-linked to nucleic acid sequences encoding the aforementioned peptides, one or more origins of replication, and, optionally, one or more selectable markers.

Once the recombinant molecules have been identified and the complex or individual proteins isolated, and a suitable host system and growth conditions have been established, using methods and systems well known within the art, the recombinant expression vectors may be propagated and amplified in-quantity. As previously discussed, expression vectors or their derivatives that can be used include, but are not limited to, human or animal viruses (e.g., vaccinia virus or adenovirus); insect viruses (e.g., baculovirus); yeast vectors; bacteriophage vectors (e.g., lambda phage); plasmid vectors and cosmid vectors.

Modification

A host cell strain may be selected that modulates the expression of inserted sequences of interest, or modifies or processes expressed peptides encoded by said sequences in the specific manner desired. In addition, expression from certain promoters may be enhanced in the presence of certain inducers in a selected host strain; thus facilitating control of the expression of a genetically-engineered peptides. Moreover, different host cells possess characteristic and specific mechanisms for the translational and post-translational processing and modification (e.g., glycosylation, phosphorylation, and the like) of expressed peptides. Appropriate cell lines or host systems may thus be chosen to ensure the desired modification and processing of the foreign peptide is achieved. For example, peptide expression within a bacterial system can be used to produce an unglycosylated core peptide; whereas expression within mammalian cells ensures “native” glycosylation of a heterologous peptide.

In a specific embodiment, the nucleic acids encoding peptides, and peptides consisting of or comprising a fragment of the aforementioned GHRH-related sequences that consists of a minimum of 6 contiguous amino acid residues of the aforementioned peptides, are provided herein. Derivatives or analogs of the aforementioned peptides include, but are not limited to, molecules comprising regions that are substantially homologous to the aforementioned peptides in various embodiments, of at least 30%, 40%, 50%, 60%, 70%, 80%, 90% or preferably 95% amino acid identity when: (i) compared to an amino acid sequence of identical size; (ii) compared to an aligned sequence in that the alignment is done by a computer homology program known within the art or (iii) the encoding nucleic acid is capable of hybridizing to a sequence encoding the aforementioned peptides under stringent (preferred), moderately stringent, or non-stringent conditions (see, e.g., supra).

Derivatives of the aforementioned peptides may be produced by alteration of their sequences by substitutions, additions or deletions that result in functionally-equivalent molecules. In an embodiment, the degeneracy of nucleotide coding sequences allows for the use of other DNA sequences that encode substantially the same amino acid sequence. In another embodiment, one or more amino acid residues within the sequence of interest may be substituted by another amino acid of a similar polarity and net charge, thus resulting in a silent alteration. Substitutes for an amino acid within the sequence may be selected from other members of the class to which the amino acid belongs. For example, nonpolar (hydrophobic) amino acids include alanine, leucine, isoleucine, valine, proline, phenylalanine, tryptophan and methionine. Polar neutral amino acids include glycine, serine, threonine, cysteine, tyrosine, asparagine, and glutamine. Positively charged (basic) amino acids include arginine, lysine and histidine. Negatively charged (acidic) amino acids include aspartic acid and glutamic acid.

Production of GHRH Analogs

The aforementioned peptides of the present invention may be produced by various methodologies known within the art. For example, the polypeptide sequences may be modified by any of numerous methods known within the art. See e.g., Sambrook, et al., 1990. Molecular Cloning: A Laboratory Manual, 2nd ed., (Cold Spring Harbor Laboratory Press; Cold Spring Harbor, N.Y.).

Isolation and Analysis of the Gene Product or Complex

Once a recombinant cell expressing an aforementioned peptide, or a fragment, homolog, analog or derivative thereof, is identified, the individual gene product or complex may be isolated and analyzed. This is achieved by assays that are based upon the physical and/or functional properties of the peptide or complex, including, but not limited to, radioactive labeling of the product followed by analysis by gel electrophoresis, immunoassay, cross-linking to marker-labeled products, and the like. The aforementioned peptide may be isolated and purified by standard methods known in the art (either from synthetic sources, natural sources or recombinant host cells expressing the peptide/peptide complex) including, but not limited to, column chromatography (e.g., ion exchange, affinity, gel exclusion, reverse-phase, high pressure, fast protein liquid, etc), differential centrifugation, differential solubility, or similar methodologies used for the purification of peptides. Alternatively, once an aforementioned peptide or its derivative is identified, the amino acid sequence of the peptide can be deduced from the nucleic acid sequence of the gene from which it was encoded. Hence, the peptide or its derivative can be synthesized by standard chemical methodologies known in the art.

In a specific embodiment, an aforementioned peptide (whether produced by recombinant DNA techniques, chemical synthesis methods, or by purification from native sources) is made up from peptides, or fragments, analogs or derivatives thereof, that, as their primary amino acid, contain sequences substantially as depicted in Formula X, as well as peptide substantially homologous thereto.

Manipulations of the Sequences

Manipulations of the sequences included within the scope of the invention may be made at the peptide level. Included within the scope of this disclosure is the aforementioned peptide, or fragments, derivatives, fragments or analogs, that is differentially modified during or after translation or synthesis (e.g., by glycosylation, acetylation, phosphorylation, amidation, derivatization by known protecting/blocking groups, proteolytic cleavage, linkage to an antibody molecule or other cellular ligand, and the like). Any of the numerous chemical modification methodologies known within the art may be utilized including, but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin, etc. In a specific embodiment, sequences of an aforementioned peptide are modified to include a fluorescent label. In another specific embodiment, an aforementioned peptide is modified by the incorporation of a heterofunctional reagent, wherein such heterofunctional reagent may be used to cross-link the members of the complex.

Production of Peptides-Expression from Tissue Culture Cells

In one embodiment, we contemplate a method of producing any one of the polypeptides set forth in Formula X, comprising culturing a cell that contains any one nucleic acid sequence encoding any one of the polypeptides set forth in Formula X under conditions permitting the production of the polypeptide, and recovering the polypeptide from the culture medium or cell culture. Any method known in the art is contemplated for steps needed for production of the peptides including, but not limited to: culturing a cell of choice in an appropriate media; introducing a nucleic acid encoding a peptide of the invention; expressing the peptide from the nucleic acid; secreting the peptide into the culture medium, recovering the peptide from the cell or the culture medium, and purifying the peptide.

In another one embodiment, we contemplate a method of producing any one or more peptide of the peptides comprising Formula X by introducing a polynucleotide which encodes, upon expression, for any peptide of Formula X into a cell or introducing a peptide coding sequence by homologous recombination into a cell, such that the endogenous regulatory sequence regulates expression of a recombinant peptide gene, to make a peptide production cell; and culturing the peptide production cell under culture conditions which result in expression of the peptide.

Cells so treated may then be introduced in vivo for therapeutic purposes by any method known in the art, including, but not limited to, implantation or transplantation of cells into a host subject, wherein the cells may be “naked” or encapsulated prior to implantation. Cells may be screened prior to implantation for various characteristics including, but not limited to, the level of peptide secreted, stability of expression, and the like.

Chemical Synthesis

Complexes of analogs and derivatives of an aforementioned peptide can be chemically synthesized. For example, a peptide corresponding to a portion of an aforementioned peptide that comprises the desired domain or that mediates the desired activity in vitro, may be synthesized by use of a peptide synthesizer. In cases where natural products are suspected of being mutant or are isolated from new species, the amino acid sequence of an aforementioned protein isolated from the natural source, as well as those expressed in vitro, or from synthesized expression vectors in vivo or in vitro, may be determined from analysis of the DNA sequence, or alternatively, by direct sequencing of the isolated protein. An aforementioned peptide may also be analyzed by hydrophilicity analysis that can be utilized to identify the hydrophobic and hydrophilic regions of the peptides, thus aiding in the design of substrates for experimental manipulation, such as in binding experiments, antibody synthesis, etc. Secondary structural analysis may also be performed to identify regions of an aforementioned peptide that assume specific structural motifs. Manipulation, translation, secondary structure prediction, hydrophilicity and hydrophobicity profiles, open reading frame prediction and plotting, and determination of sequence homologies, can be accomplished using computer software programs available in the art. Other methods of structural analysis including, but not limited to, X-ray crystallography; mass spectroscopy and gas chromatography and computer modeling may also be employed.

Methodologies for Screening

Methodologies for screening an aforementioned peptide, as well as derivatives, fragments and analogs thereof, for the ability to alter and/or modulate cellular functions, particularly those functions in which an aforementioned peptide have been implicated are provided. These functions include, but are not limited to, weight control; muscle wasting; regulation of metabolism; control of signal transduction; and pathological processes, as well as various other biological activities (e.g., binding to antibody against an aforementioned peptide, and the like). The derivatives, fragments or analogs that possess the desired immunogenicity and/or antigenicity may be utilized in immunoassays, for immunization, for inhibition of the activity of an aforementioned peptide, etc. For example, derivatives, fragments or analogs that retain, or alternatively lack or inhibit, a given property of interest may be utilized as inducers, or inhibitors, respectively, of such a property and its physiological correlates. Derivatives, fragments and analogs of an aforementioned peptide may be analyzed for the desired activity or activities by procedures known within the art.

Assays

Methodologies that are well-known within the art (e.g., immunoassays, nucleic acid hybridization assays, biological activity assays, and the like) may be used to determine whether one or more aforementioned peptides are present at either increased or decreased levels, or are absent, within samples derived from patients suffering from a particular disease or disorder, or possessing a predisposition to develop such a disease or disorder, as compared to the levels in samples from subjects not having such disease or disorder or predisposition thereto.

Accordingly, in specific embodiments, diseases and disorders that involve increased/decreased levels of activity of one or more GHRH or GHRH variant peptides may be treated with the GHRH peptides described herein, or their ability to respond to said peptides may be screened for, by quantitatively ascertaining increased/decreased levels of: (i) the one or more aforementioned peptides; (ii) the mRNA encoding an aforementioned peptide (iii) the functional activity or (iv) modulation of body weight homeostasis, following administration of the peptides.

Kits for diagnostic use that include one or more containers containing an antibody and, optionally, a labeled binding partner to said antibody are provided. The label incorporated into the antibody may include, but is not limited to, a chemiluminescent, enzymatic, fluorescent, calorimetric or radioactive moiety. In another embodiment, kits for diagnostic use that include one or more containers containing modified or unmodified nucleic acids that encode, or alternatively, that are the complement to, an aforementioned peptide and, optionally, a labeled binding partner to said nucleic acids, are also provided. In an alternative embodiment, the kit may include, in one or more containers, a pair of oligonucleotide primers (e.g., each 6-30 nucleotides in length) that are capable of acting as amplification primers for polymerase chain reaction, ligase chain reaction, cyclic probe reaction, and the like, or other methods known within the art. The kit may, optionally, further comprise a predetermined amount of a purified aforementioned peptide, or nucleic acids thereof, for use as a diagnostic, standard, or control in the aforementioned assays.

Therapeutic Uses and Biological Activity

The peptides described herein are expected to exhibit one or more of the uses or biological activities (including those associated with assays cited herein) identified below. Uses or activities described for peptides described herein may be provided by administration or use of such peptides or by administration or use of polynucleotides encoding such peptides (such as, for example, in gene therapies or vectors suitable for introduction of DNA).

Research Uses and Utilities

The peptides described herein may be used in assay to determine biological activity, including in a panel of multiple peptides for high-throughput screening; to raise antibodies or to elicit another immune response; as a reagent (including the labeled reagent) in assays designed to quantitatively determine levels of the peptides (or its receptor) in biological fluids; as markers for tissues in which the corresponding peptides are most biologically active (either constitutively or at a particular stage of tissue differentiation or development or in a disease state); and, of course, to isolate correlative receptors. Where the peptide binds or potentially binds to another protein (such as, for example, in a receptor-ligand interaction), the peptide can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Disorders

Diseases and disorders that are characterized by increased (relative to a subject not suffering from said disease or disorder) levels or biological activity may be treated with therapeutics that antagonize (i.e., reduce or inhibit) activity. Therapeutics that antagonize activity may be administered in a therapeutic or prophylactic manner. Therapeutics that may be utilized include, but are not limited to, (i) the aforementioned peptide, or analogs, derivatives, fragments or homologs thereof; (ii) antibodies to the aforementioned peptide; (iii) nucleic acids encoding the aforementioned peptide; (iv) administration of antisense nucleic acid and nucleic acids that are “dysfunctional” (i.e., due to a heterologous insertion within the coding sequences of coding sequences to an aforementioned peptide) are utilized to “knockout” endogenous function of an aforementioned peptide by homologous recombination; or (v) modulators (i.e., inhibitors, agonists and antagonists, including additional peptide mimetics of the aforementioned peptides or antibodies specific to the aforementioned peptides) that alter the interaction between an aforementioned peptide and its binding partner.

Diseases and disorders that are characterized by decreased (relative to a subject not suffering from said disease or disorder) levels or biological activity may be treated with therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof, or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to; immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Determination of the Biological Effect of the Therapeutic

In some embodiments, suitable in vitro or in vivo assays are utilized to determine the effect of a specific therapeutic and whether its administration is indicated for treatment of the affected tissue.

In various embodiments, in vitro assays may be performed with representative cells of the type(s) involved in the patient's disorder, to determine if a given therapeutic exerts the desired effect upon said cell type(s). Compounds for use in therapy may be tested in suitable animal model systems including, but not limited to rats, mice, chicken, cows, monkeys, rabbits, and the like, prior to testing in human subjects.

Gene Therapy

In some embodiments, nucleic acids comprising a sequence that encodes an aforementioned peptide, or functional derivatives thereof, are administered to modulate homeostasis of body weight and adipose tissue mass by way of gene therapy. In more specific embodiments, a nucleic acid or nucleic acids encoding an aforementioned peptide, or functional derivatives thereof, are administered by way of gene therapy. Gene therapy refers to therapy that is performed by the administration of a specific nucleic acid to a subject. In this embodiment, the nucleic acid produces its encoded peptide(s), which then serve to exert a therapeutic effect by modulating function of an aforementioned disease or disorder. Any of the methodologies relating to gene therapy available within the art may be used in the practice of the embodiments disclosed herein.

In an embodiment, the therapeutic includes a nucleic acid that is part of an expression vector expressing both of the aforementioned peptides, or fragments, derivatives or analogs thereof, within a suitable host. In a specific embodiment, such a nucleic acid possesses a promoter that is operably-linked to coding region(s) of an aforementioned peptide. Said promoter may be inducible or constitutive, and, optionally, tissue-specific. In another embodiment, a nucleic acid molecule is used in which coding sequences (and any other desired sequences) are flanked by regions that promote homologous recombination at a desired site within the genome, thus providing for intra-chromosomal expression of nucleic acids.

Delivery of the therapeutic nucleic acid into a patient may be either direct (i.e., the patient is directly exposed to the nucleic acid or nucleic acid-containing vector) or indirect (i.e., cells are first transformed with the nucleic acid in vitro, then transplanted into the patient). These two approaches are known, respectively, as in vivo or ex vivo gene therapy. In an embodiment, a nucleic acid is directly administered in vivo, where it is expressed to produce the encoded product. This may be accomplished by any of numerous methods known in the art including, but not limited to, constructing said nucleic acid as part of an appropriate nucleic acid expression vector and administering the same in a manner such that it becomes intracellular (e.g., by infection using a defective or attenuated retroviral or other viral vector); directly injecting naked DNA; using microparticle bombardment (e.g., a “Gene Gun®; Biolistic, DuPont); coating said nucleic acids with lipids; using associated cell-surface receptors/transfecting agents; encapsulating in liposomes, microparticles, or microcapsules; administering it in linkage to a peptide that is known to enter the nucleus; or by administering it in linkage to a ligand predisposed to receptor-mediated endocytosis, which can be used to “target” cell types that specifically express the receptors of interest, etc.

In another embodiment, a nucleic acid-ligand complex may be produced in which the ligand comprises a fusogenic viral peptide designed so as to disrupt endosomes, thus allowing the nucleic acid to avoid subsequent lysosomal degradation. In yet another embodiment, the nucleic acid may be targeted in vivo for cell-specific endocytosis and expression, by targeting a specific receptor. Alternatively, the nucleic acid may be introduced intracellularly and incorporated within a host cell genome for expression by homologous recombination.

In another embodiment, a viral vector that contains nucleic acids encoding an aforementioned peptide is utilized. For example, retroviral vectors may be employed that have been modified to delete those retroviral-specific sequences that are not required for packaging of the viral genome, with its subsequent integration into host cell DNA. Nucleic acids may be cloned into a vector that facilitates delivery of the genes into a patient. Additionally, adenovirus may be used as an especially efficacious “vehicle” for the delivery of genes to the respiratory epithelia. Other targets for adenovirus-based delivery systems are liver, central nervous system, endothelial cells, and muscle. Adenoviruses also possess advantageous abilities to infect non-dividing cells. Adenovirus-associated virus (AAV) has also been proposed for use in gene therapy.

An additional approach to gene therapy involves transferring a gene into cells in in vitro tissue culture by such methods as electroporation, lipofection, calcium phosphate-mediated transfection, viral infection, or the like. Generally, the methodology of transfer includes the concomitant transfer of a selectable marker to the cells. The cells are then placed under selection pressure (e.g., antibiotic resistance) so as to facilitate the isolation of those cells that have taken up, and are expressing, the transferred gene. Those cells are then delivered to a patient. In a specific embodiment, prior to the in vivo administration of the resulting recombinant cell, the nucleic acid is introduced into a cell by any method known within the art including, but not limited to: transfection, electroporation, microinjection, infection with a viral or bacteriophage vector containing the nucleic acid sequences of interest, cell fusion, chromosome-mediated gene transfer, microcell-mediated gene transfer, spheroplast fusion, and similar methodologies that ensure that the necessary developmental and physiological functions of the recipient cells are not disrupted by the transfer. The chosen technique should provide for the stable transfer of the nucleic acid to the cell, such that the nucleic acid is expressible by the cell. Preferably, said transferred nucleic acid is heritable and expressible by the cell progeny.

In some embodiments, the resulting recombinant cells may be delivered to a patient by various methods known within the art including, but not limited to, injection of epithelial cells (e.g., subcutaneously), application of recombinant skin cells as a skin graft onto the patient, and intravenous injection of recombinant blood cells (e.g., hematopoietic stem or progenitor cells). The total amount of cells that are envisioned for use depend upon the desired effect, patient state, and the like, and may be determined by one skilled within the art.

Cells into which a nucleic acid can be introduced for purposes of gene therapy encompass any desired, available cell type, and may be xenogeneic, heterogeneic, syngeneic, or autogeneic. Cell types include, but are not limited to, differentiated cells such as epithelial cells, endothelial cells, keratinocytes, fibroblasts, muscle cells, hepatocytes and blood cells, or various stem or progenitor cells, in particular embryonic heart muscle cells, liver stem cells, hematopoietic stem or progenitor cells, e.g., as obtained from bone marrow, umbilical cord blood, peripheral blood, fetal liver, and the like. In an embodiment, the cells utilized for gene therapy are autologous to the patient.

In an embodiment in which recombinant cells are used in gene therapy, stem or progenitor cells that can be isolated and maintained in vitro may be utilized. Such stem cells include, but are not limited to, hematopoietic stem cells (HSC), stem cells of epithelial tissues, and neural stem cells. With respect to HSCs, any technique that provides for the isolation, propagation, and maintenance in vitro of HSC may be used in this embodiment. As previously discussed, the HSCs utilized for gene therapy are, preferably but not by way of limitation, autologous to the patient. When used, non-autologous HSCs are, preferably but not by way of limitation, utilized in conjunction with a method of suppressing transplantation immune reactions of the future host/patient. In an embodiment, HSCs may be highly enriched (or produced in a substantially-pure form), by any techniques known within the art, prior to administration to the patient.

Administration and Dosing

Some embodiments include methods of treatment and prophylaxis by the administration to a subject of a pharmaceutically-effective amount of a therapeutic agent as described herein. In an embodiment, the therapeutic is substantially purified and the subject is a mammal, and most preferably, human.

Pharmaceutical compositions. A GHRH peptide as described herein (derived from whatever source defined herein, including without limitation from synthetic, recombinant and non-recombinant sources) may be used in a pharmaceutical composition when combined with a pharmaceutically acceptable carrier. Such compositions comprise a therapeutically-effective amount of the GHRH peptide, and a pharmaceutically acceptable carrier. Such a composition may also include (in addition to peptide and a carrier) diluents, fillers, salts, buffers, stabilizers, solubilizers, and other materials well known in the art. As utilized herein, the term “pharmaceutically acceptable” means a non-toxic material that does not interfere with the effectiveness of the biological activity of the active ingredient(s), approved by a regulatory agency of the Federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils. The characteristics of the carrier will depend on the route of administration.

A peptides described herein may be active in multimers (e.g., heterodimers or homodimers) or complexes with itself or other peptides. As a result, pharmaceutical compositions may include a GHRH peptide described herein in such multimeric or complexed form.

Methods of Administration

Methods of administration include, but are not limited to, intradermal, intramuscular, intraperitoneal, intravenous, subcutaneous, intranasal, epidural, and oral routes. The GHRH peptides described herein may be administered by any convenient route, for example by infusion or bolus injection, by absorption through epithelial or mucocutaneous linings (e.g., oral mucosa, rectal and intestinal mucosa, etc.) and may be administered together with other biologically-active agents. Administration can be systemic or local. In addition, it may be advantageous to administer the GHRH peptides into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the GHRH peptides locally to the area in need of treatment; this may be achieved by, for example, and not by way of limitation, local infusion during surgery, topical application, by injection, by means of a catheter, by means of a suppository, or by means of an implant.

In one specific embodiment of the invention, subcutaneous administration of GHRH peptides is contemplated and preferred.

Delivery

Various delivery systems are known and can be used to administer GHRH peptides described herein including, but not limited to: (i) encapsulation in liposomes, microparticles, microcapsules; (ii) recombinant cells capable of expressing the GHRH peptides; (iii) receptor-mediated endocytosis; (iv) construction of therapeutic nucleic acid as part of a retroviral or other vector, and the like. In one embodiment, the GHRH peptides may be delivered in a vesicle, in particular a liposome. In a liposome, the GHRH peptide is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,837,028; and U.S. Pat. No. 4,737,323, all of which are incorporated herein by reference. In yet another embodiment, the GHRH peptides may be delivered in a controlled release system including, but not limited to: a delivery pump and a semi-permeable polymeric material. Additionally, the controlled release system can be placed in proximity of the therapeutic target (e.g., the brain), thus requiring only a fraction of the systemic dose.

In an embodiment, where the therapeutic is a nucleic acid encoding a peptide, the therapeutic nucleic acid may be administered in vivo to promote expression of its encoded peptide, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide which is known to enter the nucleus, and the like. Alternatively, a nucleic acid therapeutic can be introduced intracellularly and incorporated within host cell DNA for expression, by homologous recombination.

Dosage.

The term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of the therapeutic which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of peptide of the present invention with which to treat each individual patient. Initially, the attending physician will administer low doses of peptide and observe the patient's response. Larger doses of peptide may be administered until the optimal therapeutic effect is obtained for the patient, and at that point the dosage is not increased further. However, suitable dosage ranges for intravenous administration of the therapeutics are generally about 5-500 micrograms (μg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient.

Duration

The duration of intravenous therapy using the pharmaceutical composition will vary, depending on the severity of the disease being treated and the condition and potential idiosyncratic response of each individual patient. It is contemplated that the duration of each application of the peptide of the present invention will be in the range of 12 to 24 hours of continuous intravenous administration. Ultimately the attending physician will decide on the appropriate duration of intravenous therapy using the pharmaceutical composition.

Pharmaceutical Pack or Kit.

Embodiments also include a pharmaceutical pack or kit, including one or more containers filled with one or more of the ingredients of the pharmaceutical compositions and therapeutics. Optionally associated with such container(s) may be a notice in the form prescribed by a governmental agency regulating the manufacture, use or sale of pharmaceuticals or biological products, which notice reflects approval by the agency of manufacture, use or sale for human administration.

Gene Therapy.

Polynucleotides as described herein may also be used for gene therapy. Gene therapy refers to therapy that is performed by the administration of a specific nucleic acid to a subject. Delivery of the therapeutic nucleic acid into a mammalian subject may be either direct (i.e., the patient is directly exposed to the nucleic acid or nucleic acid-containing vector) or indirect (i.e., cells are first transformed with the nucleic acid in vitro, then transplanted into the patient). These two approaches are known, respectively, as in vivo or ex vivo gene therapy. Polynucleotides may also be administered by other known methods for introduction of nucleic acids into a cell or organism (including, without limitation, in the form of viral vectors or naked DNA). Any of the methodologies relating to gene therapy available within the art may be used.

Cultured Cells.

Cells may be cultured ex vivo in the presence of GHRH peptides described herein in order to proliferate or to produce a desired effect on or activity in such cells. Treated cells can then be introduced in vivo for therapeutic purposes.

In some embodiments, a method of identifying a modulator and/or potential modulator of body mass homeostasis in situ is provided, that includes: contacting a cell with the presence or absence of peptide, the peptide including any one or more of the peptides of Formula X; determining the level of effect in cells so contacted compared to cells not so contacted; wherein when an increase or decrease in desired effect is determined in the presence of the peptide relative to the absence of the peptide, the peptide is identified as a potential modulator of body mass homeostasis.

In another embodiment, a method of identifying a modulator and/or potential modulator of body mass homeostasis in vivo includes: administering to a test animal doses of at least one peptide and comparing said animal to a placebo control animal over a prescribed time period, wherein the peptide includes any one or more of the peptides of Formula X; determining the level of modulation in body homeostasis of the test animal compared to the control during the prescribed time period; wherein when an increase or decrease in desired effect is determined in the presence of the peptide relative to the absence of the peptide, the peptide is identified as a potential modulator of body mass homeostasis.

Pharmaceutical Composition

Another embodiment is related to a pharmaceutical composition comprising a pharmaceutically effective amount of a GHRH analog, functional derivative or salt thereof as described hereinabove, and a pharmaceutically acceptable carrier.

The term “composition” as used herein is intended to encompass a product including the GHRH peptides described herein in the desired amounts. By “pharmaceutically acceptable”, it is meant that the carrier, diluent or excipient must be compatible with the GHRH peptide(s) of the formulation and can be administered into a host without adverse effects. Suitable pharmaceutically acceptable carriers known in the art include, but are not limited to, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, lactose, colors and the like. A preferable pharmaceutically acceptable carrier contemplated is a saline solution, such as sodium chloride, preferably used at 0.9% or lactose used for the preparation of dry powder formulations intended for inhalation.

Methods of Use

Further embodiments relate to the use of GHRH peptides described herein or a pharmaceutical composition comprising same for the specific stimulation of in vivo release of GH and IGF-1, as well as for the preparation of a drug in the treatment of GH deficiency-related or endogenous GH resistance conditions. By “treatment”, it is meant both therapeutic treatment and prophylactic or preventative measures. Those in need of treatment include those already with the disorder or GH deficiency/resistance as well as those prone to have the disorder or GH deficiency/resistance, or those in which the disorder or GH deficiency/resistance is to be prevented.

The expression “specific stimulation of in vivo release of GH” refers to the action of GHRH peptides described herein which activate GH release and in turn IGF-1 release by direct binding to the GHRH receptor, but which do not activate GH release by direct binding to other receptor molecules, in a sample containing a mixed population of receptors.

GH deficiency-related conditions of the present invention encompass but are not limited to the following: hypothalamic pituitary dwarfism, burns, osteoporosis, renal failure (e.g., chronic kidney disease), non-union bone-fracture, acute/chronic debilitating illness or infection, wound healing, post-surgical problems, lactation failure, infertility in women, cachexia in cancer patients, anabolic and/or catabolic problems, T-cell immunodeficiencies, neurodegenerative conditions, GHRH receptor-dependent tumors, aging, sleep disorders, and muscle wasting diseases. As used herein, muscle wasting diseases could be any one of the following: sarcopenia, frailty in the elderlies, HIV and cancer. More specifically, use of the present pharmaceutical composition could be aimed at cancer patients who present side effects related to chemotherapy and radiotherapy.

In yet another embodiment, methods for initiating GHRH-induced biological actions in a mammal are contemplated. One such method includes administering, to the mammal, an effective amount of a GHRH analog, a functional derivative of said analog or a pharmaceutically acceptable salt thereof, as defined herein, or of a pharmaceutical composition as defined above.

Expressions such as “GHRH-induced biological activity”, and the like, as used herein include the following: regulation of sleep, regulation of food-intake and increase in protein synthesis. The increase in protein synthesis observed following GHRH analog administration, could translate into an increase in muscle mass or an increase in milk production, among others.

As used herein the term “mammal” refers to any animal classified as a mammal, including humans, domestic and farm animals, and zoo, sports, or pet animals, such as dogs, horses, cats, cows, pigs, etc, in whom modulation of GHRH receptor activity is desired. “Modulation”, as used herein, is intended to encompass agonism, and/or partial agonism.

The term “effective amount” means the amount of GHRH analog that will elicit the biological or clinical response of a tissue, system, animal or human that is being sought by the researcher, veterinarian, medical doctor or other clinician. In other words, such an effective amount of a compound for treating a particular disease is an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. The terms “administration of a” and “administering a” compound should be understood to mean providing a GHRH analog as described herein or a composition of the GHRH analog to the individual in need of treatment.

The GHRH analog and the composition of the GHRH analog may be given to a mammal through various routes of administration. For instance, the composition may be administered in the form of sterile injectable preparations, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, or by intramuscular injection or by infusion. The GHRH analog and the composition of the GHRH analog may also be formulated as creams, ointments, lotions, gels, drops, suppositories, sprays, liquids or powders for topical administration. They may also be administered into the airways of a subject by way of a pressurized aerosol dispenser, a nasal sprayer, a nebulizer, a metered dose inhaler, a dry powder inhaler, or a capsule. Suitable dosages will vary, depending upon factors such as the amount of each of the components in the composition, the desired effect (fast or long term), the disease or disorder to be treated, the route of administration, the bioavailability, and the age and weight of the mammal to be treated. In any event, for administering the GHRH analog and compositions of the GHRH analogs, methods well known in the art may be used.

### EXAMPLES

The following examples illustrate the wide range of potential applications of the present invention and are not intended to limit its scope. Modifications and variations can be made therein without departing from the spirit and scope of the invention. Although any methods and materials similar or equivalent to those described herein can be used in the practice for testing the present invention, the preferred methods and materials are described.

### Example 1

### Initial Selection of GHRH Analogs Based Upon In Vitro Data from GHRH Receptor Binding Affinity

Initial selection of a candidate from the original 14 polysubstituted GHRH analogs described in the U.S. Pat. No. 5,854,216 was based upon in vitro data on receptor affinity in 2-month old male Sprague Dawley rat anterior pituitary preparations. The embodiments are based on the affinity of selected GHRH analogs for the human GHRH receptor (hGHRH-R) in baby hamster kidney (BHK) cells transfected with hGHRH-R, and on resistance to proteolysis in rat serum, human plasma or human serum. More precisely, the preferred drug candidates were selected, as compared to hGHRH(1-29)-NH₂, for: i-their increased relative binding affinity to hGHRH(1-44)-NH₂ binding sites in rat anterior pituitary in vitro as well as to hGHRH-R in BHK-expressing cells in vitro; and ii-their relative resistance to proteolysis in vitro.

As can be noted from Table 1 below, the relative binding affinity of the synthetic peptides with the rat GHRH receptor is not predictive of the relative binding affinity with the human receptor. As will be noted, from this point forward, GHRH analogs as presented in Table 1 will be referred to as GHRH analogs #1 to 5.

TABLE 1

| | | Relative binding | Relative binding affinity | |
|---|---|---|---|---|
| | | affinity in rat | in hGHRH-R BHK- | Relative resistance to |
| No. | Structure | anterior pituitary*† | expressing cells*^{†} | proteolysis in vitro |
| 1 | [D-Ala², Ala⁸, Ala¹⁵, Lys²²] hGHRH(1- | 13.33 ± 0.31 | 499 ± 234 | 1.87 |
| | 29)-NH₂ | | | |
| 2 | [Ala⁸, Ala⁹, Ala¹⁵, Ala²²] hGHRH(1- | 7.74 ± 3.49 | 3.70 ± 0.52 | 1.81 |
| | 29)-NH₂ | | | |
| 3 | [D-Ala², D-Tyr¹⁰, Lys²²] hGHRH(1- | 4.90 ± 2.70 | 239 ± 55 | 2.25 |
| | 29)-NH₂ | | | |
| 4 | [D-Ala², Ala⁸, D-Tyr¹⁰, Ala¹⁵, D-Lys²¹, | 5.00 ± 0.91 | 0.05 ± 0.01 | 6.06 |
| | Lys²²] hGHRH(1-29)-NH₂ | | | |
| 5 | [D-Ala², D-Tyr¹⁰, D-Ala¹⁵, Lys²²] | 1.04 ± 0.40 | 939 ± 249 | 3.13 |
| | hGHRH(1-29)-NH₂ | | | |

Priority selection based on the expected theoretical combined effects of receptor affinity and in vitro resistance to proteolysis on the overall bioactivity of GHRH analogs in rat anterior pituitary membrane preparations and rat serum, respectively, and of receptor affinity in BHK cell membrane preparations. GHRH analog numbers in Table 1 correspond to numbers 13, 11, 7, 14 and 8 in Table 11 on pages 27-28 of the U.S. Pat. No. 5,854,216, respectively.
*values compared to hGHRH(1-29)-NH₂;
†use of [¹²⁵I-Tyr¹⁰]hGHRH(1-44)-NH₂ as a radioligand in structure-affinity studies.

### Example 2

### Processing of the Native GHRH and GHRH Analogs—Experimental Assays

Competitive Binding Assay:

¹²⁵I-GHRH binding assay was performed using [¹²⁵I-Tyr¹⁰]hGHRH(1-44)NH₂ as a radioligand. Competition experiments were done in BHK (baby hamster kidney) 570 cell membrane preparations (25 μg of protein/assay tube) with increasing concentrations (0-1000 nM) of human(h)GHRH(1-29)NH₂, hGHRH(1-44)NH₂ or GHRH analogs, in a total volume of 300 μl of 50 mM Tris-acetate buffer (pH 7.4), containing 5 mM of MgCl₂, 5 mM EDTA and 0.42% BSA. Non specific binding was determined in presence of 1 μM hGHRH(1-29)NH₂. Incubation was carried out at equilibrium (23° C., 60 min) and stopped by centrifugation (12,000 g, 5 min, at 4° C.). The radioactivity content in pellets was determined by gamma counting. The affinity of hGHRH(1-29) NH₂ was tested in each experiment to assess the validity of the assay and determine the relative affinity of the analogs. The ligand computerized program was used to analyze competition curves of GHRH analogs reported in Tables 2 and 3 and to determine their IC50.

In Vitro Proteolysis Assay in Serum and in Plasma

10 μl of a 300 μM solution of hGHRH (1-29)NH₂ or of a GHRH analog was solubilized in dimethysulfoxide (DMSO) and incubated in one of the following conditions: a-190 μl serum (1/100 dilution in picopure water) from 2-month-old male Sprague Dawley rats, at 37° C. for 0, 8, 15, 30 or 60 min, in polypropylene tubes; b-190 μl of human healthy volunteer plasma (from Human Whole Blood Na EDTA, males, drug free (Algorithme Pharma Inc.); project: MTL-P2-155; Lot: MTLP2155-01, supplied by LAB Dev Int); and c-190 μl of human healthy volunteer pooled serum, Lot: X409 (supplied by LAB Dev Int), at 37° C. for 0, 60, 120, 180 or 420 min, in polypropylene tubes. Proteolysis was stopped by adding 800 μl of ice-cold stop buffer (potassium-phosphate buffer, acidified to pH 0.8 with trifluoroacetic acid (TFA) and boiling 5 min (rat serum only). After centrifugation (12000 g, 5 min, 4° C.) (rat serum only), serum-peptide mixtures were passed through a conditioned Sep-Pak C-18 cartridge to extract native GHRH or a GHRH analog residual concentrations from serum proteins. The native GHRH or the analog was eluted in 2 ml of 50% acetonitrile-0.01% TFA/50% 0.01% aqueous TFA. 200 μl of extracted peptide, representing 1 μg of GHRH or analog at time 0, was quantified by analytical HPLC, using one μ-Bondapak C18 column (10 μm particle size, 0.39×15 cm) (rat serum) or two C18 columns in series (human serum and plasma) and a binary solvent system composed of NaClO₄ 0.01 M, pH 2.5 and acetonitrile. A linear gradient from 30 to 60% acetonitrile over 45 min (rat serum) or 30 to 50% (human serum and plasma) was used. Elution of intact peptide was monitored at 214 nm and residual concentration determined by assessment of peak surface areas.

In Vivo Administration of Native GHRH or GHRH Analog

In order to assess such use of the GHRH analogs, the following experiments were undertaken. More specially, the goal was to assess the pharmacodynamic and pharmacokinetic profiles and acute toxicity of GHRH analog #5 when administered once by subcutaneous or intravenous injection to female Sprague-Dawley rats followed by a 14-day observation period and the pharmacodynamic profile in a male Beagle dog when the GHRH analog was administered at escalating doses to the same dog by subcutaneous injection with at least 2-day washout period. The above GHRH analog is a variation of a synthetic acetate salt of an amidated synthetic 29-amino acid peptide that corresponds to the amino-terminal segment of the naturally-occurring human growth hormone—releasing hormone (GHRH) with four amino acid substitutions in positions 2, 10, 15, and 22.

The ability of human GHRH analog #5 (human [D-Ala2, D-Tyr10, D-Ala15, Lys22] GHRH (1-29)NH₂ analog) to stimulate GH secretion was studied in adult female rats (26-34 weeks at onset of treatment) and in a male Beagle dog.

In Vivo Administration into Rats

Human GHRH analog #5 in 0.9% sodium chloride for injection USP was administered once either by intravenous (IV) or subcutaneous (SC) injection to female rats followed by a 14-day observation period, as shown in Table 2. Prior to administration, all dosing formulations were filtered using a 0.22 μm filter to ensure sterility. The actual amount of GHRH analog #5 administered was calculated and adjusted based on the animal's most recent body weight. Dosing started at approximately the same time each day, commencing at 9:00 am ±30 minutes.

TABLE 2
In vivo administration of GHRH analog # 5 to female rats.

| Treatment | Dose Level | Dose Conc. | Route of | No. of |
|---|---|---|---|---|
| Group | (mg/kg) | (mg/ml) | Administration | Animals |
| 1 (Neg. | 0 | 0 | SC | 4 |
| Control*) | | | | |
| 2 | 0.001 | .001 | SC | 4 |
| 3 | 0.01 | .01 | SC | 4 |
| 4 | 0.03 | .03 | SC | 4 |
| 5 | 0.1 | 0.1 | SC | 4 |
| 6 | 0.3 | 0.3 | SC | 4 |
| 7 | 1 | 1 | SC | 4 |
| 8 | 3 | 3 | SC | 4 |
| 9 | 0.001 | 0.001 | IV | 4 |
| 10 | 0.03 | 0.03 | IV | 4 |
| 11 | 3 | 3 | IV | 4 |
| 12 (Pos. | 0.03 | 0.03 | IV | 4 |
| Control**) | | | | |

*Negative control (Group 1) animals only received the vehicle (NaCl).
**Positive control (Group 12) animals received hGHRH(1-44) only.

For pharmacodynamic investigations, blood samples (approximately 1.3 ml) were collected from 2 animals per group per time point (maximum 3 time points/animal) via a jugular venipuncture at the following time points: pre-dose, 4, 10, 15, 45 minutes and 5 hours post dosing. All blood samples were collected into potassium EDTA tubes and centrifuged under refrigeration (2 to 8° C., 1500 g for 10 minutes).

Rat Growth Hormone Determination
Plasma GH was determined by Linco Diagnostic Services using their own kit. Linco's Rat Growth Hormone radioimmunoassay kit (RIA) (RGH-45HK) is intended for the quantitative determination of Rat Growth Hormone in serum, plasma, and tissue culture media. It is a completely homologous assay since the antibody was raised against recombinant Rat Growth Hormone and both the tracer and the standard are prepared with the same recombinant Rat Growth Hormone. The kit includes standards, antibody, tracer, quality controls, precipitating reagents and buffer necessary to complete a RIA. The assay was conducted under the following conditions: overnight; equilibrium incubation at room temperature; sample volume: 100 μl serum, plasma, or cell culture media. The label used was 125I-Rat Growth Hormone (20,000 CPM/tube).

The performance of the assay was:

ED80=1.0±0.1 ng/ml

ED50=4.7±0.2 ng/ml

ED20=23.1±0.7 ng/ml

Finally, the specificity of the assay was the following:

Rat Growth Hormone 100%;

Rat Prolactin <0.1%;

Porcine Growth Hormone <0.5%;

Human Growth Hormone <0.1%.

In vivo administration into a male Beagle dog

Human GHRH analog #5, in 0.9% sodium chloride for injection USP, was administered on days 3, 5 and 8 at dose levels of 0.01, 0.1, and 1 mg/kg body weight, respectively by subcutaneous (SC) injection to an approximately 8-month old male dog as shown in Table 3. On Day 1, the dog received the control (vehicle) article and on Day 11, the animal received the positive control, hGHRH (1-44)NH₂ at a dose level of 0.01 mg/kg. Prior to administration, all dosing formulations were filtered using a 0.22 μm filter to ensure sterility. The actual amount of GHRH analog #5 administered was calculated and adjusted based on the animal's most recent body weight. Dosing started at approximately the same time each day, commencing at 9:00 am ±30 minutes.

TABLE 3
In vivo administration of GHRH analog # 5 to a male Beagle dog.

| | Dose Level | Dose Conc. | Route of | Animal |
|---|---|---|---|---|
| Day | (mg/kg) | (mg/ml) | Administration | Number |
| 1 | 0 | 0 | SC | 1002A |
| (Neg. Control*) | | | | |
| 3 | 0.01 | 0.01 | SC | 1002A |
| 5 | 0.1 | 0.1 | SC | 1002A |
| 8 | 1.00 | 1.00 | SC | 1002A |
| 11 | 0.01 | 0.01 | SC | 1002A |
| (Pos. Control**) | | | | |

*Negative control: the animal received only the vehicle (NaCl).
**Positive control (Day 11): the animal received hGHRH(1-44) only.

For pharmacodynamic investigations, blood samples (approximately 1.0 ml) were collected from the dog on each treatment day via a jugular venipuncture at the following time points: pre-dose, 7, 15, 22, 30, 45, and 60 minutes post dosing. All blood samples were collected into potassium EDTA tubes and centrifuged under refrigeration (2 to 8° C., 1500 g for 10 minutes).

Canine Growth Hormone Determination

Plasma GH was determined by Linco Diagnostic Services using their own kit. Linco's Porcine/Canine Growth Hormone radioimmunoassay kit (RIA) (PGH-46HK) has been developed to quantitate Growth Hormone in plasma, serum, and tissue culture media. It is a completely homologous assay since the antibody was raised against recombinant Porcine Growth Hormone and both the standard and tracer are prepared with recombinant Porcine Growth Hormone. Since the amino acid sequences of Porcine Growth Hormone and Canine Growth Hormone are identical, this assay developed for Porcine Growth Hormone measures Canine Growth Hormone levels with equal efficiency. All components are included (standards, antibody, tracer, quality controls, precipitating reagents and buffer) necessary to complete a RIA. The assay was conducted under the following conditions: overnight; equilibrium incubation at room temperature; sample volume: 100 μl serum, plasma, or cell culture media. The label used was 125I-Porcine/Canine Growth Hormone (18,000 CPM/tube).

The performance of the assay was:

ED₈₀=2.3±0.2 ng/ml

ED₅₀=9.8±0.5 ng/ml

ED₂₀=41.8±1.4 ng/ml

Finally, the specificity of the assay was the following:

Porcine Growth Hormone 100%;

Porcine Prolactin <0.1%;

Canine Growth Hormone 100%;

Human Growth Hormone <0.5%.

### Example 3

### In Vitro Proteolytic Resistance of Analogs Compared to hGHRH(1-29)NH₂ in Rat Serum

As presented in Table 4, after a 60-minute incubation period, all GHRH analogs presented significantly higher residual concentrations in comparison with hGHRH(1-29)NH₂. Moreover, the residual concentration of GHRH analog #5 was significantly higher than that of either GHRH analog 1, 2 or 3. Therefore, with the exception of GHRH analog #4, these results indicate that GHRH analog #5 exhibited the best in vitro resistance to proteolysis, using the described assay.

TABLE 4
In vitro proteolytic resistance of analogs compared to
hGHRH(1-29)NH2 in rat serum.

| | Duration of | Residual concentration |
|---|---|---|
| Compound | incubation (min) | (% of initial concentration) |
| hGHRH(1-29)NH2 | 0 | 100 ± 0 |
| (n = 19) | 8 | 81 ± 2 |
| | 15 | 66 ± 3 |
| | 30 | 43 ± 2 |
| | 60 | 16 ± 1 |
| GHRH analog # 1 | 0 | 100 ± 0 |
| (n = 3) | 8 | 75 ± 12 |
| | 15 | 70 ± 15 |
| | 30 | 53 ± 8 |
| | 60 | 30 ± 6 |
| GHRH analog # 2 | 0 | 100 ± 0 |
| (n = 4) | 8 | 83 ± 3 |
| | 15 | 73 ± 5 |
| | 30 | 53 ± 3 |
| | 60 | 29 ± 2 |
| GHRH analog # 3 | 0 | 100 ± 0 |
| (n = 4) | 8 | 82 ± 7 |
| | 15 | 88 ± 7 |
| | 30 | 70 ± 12 |
| | 60 | 36 ± 4 |
| GHRH analog # 4 | 0 | 100 ± 0 |
| (n = 4) | 8 | 98 ± 2 |
| | 15 | 100 ± 0 |
| | 30 | 99 ± 1 |
| | 60 | 97 ± 3 |
| GHRH analog # 5 | 0 | 100 ± 0 |
| (n = 4) | 8 | 92 ± 5 |
| | 15 | 82 ± 6 |
| | 30 | 74 ± 7 |
| | 60 | 50 ± 3 |

Values represent the mean ± SEM of 3 to 4 experiments for the GHRH analogs and the mean ± SEM of 19 experiments for hGHRH(1-29)NH2.

### Example 4

### In Vitro Proteolytic Resistance of Analogs Compared to hGHRH(1-29)NH₂ in Human Plasma and Serum

Referring now to Tables 5 and 6, one can see values of the in vitro proteolytic resistance of hGHRH(1-44)NH₂, hGHRH(1-29)NH₂ and of three GHRH analogs. This resistance is expressed as the mean residual amount of each peptide (in percentage) upon incubation times varying from 0 to 420 minutes in human plasma (Table 5) and human serum (Table 6). More specifically, the values represent the mean, standard deviation and standard error from the mean of 3 to 7 experiments.

As can be particularly appreciated in Table 5, with regard to the native form of GHRH, incubation times varying from 180 to 420-minute led to a significant decrease in the mean residual amount of said peptides. In contrast, after a 180-minute incubation, all three (3) analogs still presented relatively high mean residual amounts (68 to 81%). Moreover, even after a 420-minute incubation, GHRH analog #5 still presented 75% of mean residual amount. Using the two-tailed unpaired Student's t test with Welch's correction, with a statistical significance established at P<0.05, a significant difference was observed between the residual amount of analogs compared to human GHRH(1-29)NH₂. Upon further statistical analysis, it was also observed that the residual amount of hGHRH(1-29)NH₂ was significantly lower in human plasma than that of anyone of GHRH analogs #1, 3 and 5 (P<0.01). However, the mean residual amount of these analogs was not significantly different from one another.

Referring now to Table 6, one can appreciate that upon a 420-minute incubation, while hGHRH(1-29)NH₂ disappeared totally, GHRH analog #5 remained at 50% of its initial concentration.

Therefore, upon incubation in both human plasma and human serum, the residual amount of the native form of GHRH was significantly lower than that of its analogs.

TABLE 5
In vitro proteolytic resistance of native GHRH and GHRH
analogues, upon incubation in human plasma.

| | | Mean | | | |
|---|---|---|---|---|---|
| | | residual | | | |
| Peptide | IT (min) | amount (%) | SD | SEM | n |
| hGHRH (1-44) NH2 | 0 | 100 | 0 | 0 | 3 |
| | 180 | 31 | 1 | 1 | 3 |
| | 420 | 3 | 5 | 3 | 3 |
| hGHRH (1-29) NH2 | 0 | 100 | 0 | 0 | 5 |
| | 60 | 53 | 7 | 4 | 4 |
| | 120 | 44 | 5 | 3 | 4 |
| | 180 | 23 | 15 | 5 | 8 |
| | 420 | 5 | 9 | 5 | 3 |
| (D-Ala-2, Ala-8, Ala-15, | 0 | 100 | 0 | 0 | 4 |
| Lys-22) hGHRH (1-29) NH₂ | | | | | |
| | 60 | 79 | 7 | 4 | 4 |
| | 120 | 63 | 7 | 4 | 4 |
| | 180 | 68 | 1 | 1 | 3 |
| (D-Ala-2, D-Tyr-10, | 0 | 100 | 0 | 0 | 4 |
| Lys-22) hGHRH (1-29) NH₂ | | | | | |
| | 60 | 87 | 10 | 5 | 4 |
| | 120 | 78 | 15 | 8 | 4 |
| | 180 | 81 | 11 | 6 | 4 |
| (D-Ala-2, D-Tyr-10, | 0 | 100 | 0 | 0 | 4 |
| D-Ala-15, Lys-22) | | | | | |
| hGHRH (1-29) NH₂ | | | | | |
| | 60 | 92 | 10 | 5 | 4 |
| | 120 | 84 | 12 | 6 | 4 |
| | 180 | 78 | 11 | 4 | 7 |
| | 420 | 75 | 3 | 2 | 3 |

IT: incubation time;
SEM: standard error from the mean;
SD: standard deviation;
n: number of experiments.

TABLE 6
In vitro proteolytic resistance of native GHRH and GHRH
analogs, upon incubation in human serum.

| | | Mean residual | | | |
|---|---|---|---|---|---|
| Peptide | IT (min) | amount (%) | SD | SEM | n |
| hGHRH (1-29) NH₂ | 0 | 100 | 0 | 0 | 3 |
| | 60 | 57 | 11 | 6 | 3 |
| | 120 | 37 | 2 | 1 | 3 |
| | 180 | 16 | 10 | 4 | 6 |
| | 420 | 0 | 0 | 0 | 3 |
| (D-Ala-2, D-Tyr-10, | 0 | 100 | 0 | 0 | 3 |
| D-Ala-15, Lys-22) | | | | | |
| hGHRH (1-29) NH₂ | | | | | |
| | 60 | 88 | 20 | 12 | 3 |
| | 120 | 76 | 8 | 5 | 3 |
| | 180 | 63 | 5 | 2 | 6 |
| | 420 | 50 | 7 | 4 | 3 |

IT: incubation time;
SEM: standard error from the mean;
SD: standard deviation;
n: number of experiments.

### Example 5

### Binding Affinity of GHRH in its Native and Analog Forms to the hGHRH Receptor

As shown in Table 7, no significant difference was observed (two-tailed unpaired Student's t test with Welch's correction, statistical significance established at P<0.05) between the IC50 of human GHRH(1-44)NH₂ and that of GHRH analog #5 indicating that this GHRH analog has an affinity at least as high as the native human GHRH(1-44)NH₂ for the human GHRH receptor.

Values represent the mean±SEM of 3 experiments performed in triplicate for the analogs and the mean±SEM of 2 experiments performed in triplicate for hGHRH(1-44) NH₂. IC50 is the concentration of peptide inhibiting 50% of 125I-GHRH specific binding as determined by the LIGAND program for analysis of competition curves.

TABLE 7
In vitro binding affinity of human GHRH analog # 5 and
hGHRH(1-44)NH₂ in BHK cell membrane preparations
expressing the human GHRH receptor.

| No. | Name of compound | IC₅₀ (pM) |
|---|---|---|
| | Human GHRH(1-44)NH2 | 5.2 ± 3.4 |
| 5 | [D-Ala2, D-Tyr10, D-Ala15, Lys22] | 1.2 ± 0.4 |
| | human GHRH(1-29)NH₂ | |

### Example 6

### In Vitro Binding Affinity of hGHRH (1-29)-NH₂ Analogs and hGHRH (1-29)-NH₂ in BHK Cell Membrane Preparations Expressing the Human GHRH Receptor and In Vitro Proteolytic Resistance of the Analogs

For the binding assay results presented in Tables 8 to 11, values represent the mean±SEM of 8 independent experiments performed in triplicate for the analogs and the mean±SEM of 4 experiments performed in triplicate for hGHRH(1-29)NH₂. IC50 is the concentration of peptide inhibiting 50% of 125I-GHRH specific binding as determined by the LIGAND program for analysis of competition curves. The relative affinity was obtained by taking the ratio IC50 of hGHRH (1-29)-NH₂/IC50 analog.

For the proteolysis assay results presented in Tables 9 to 11, values represent the mean±SEM of 3 to 5 independent experiments.

As shown in following Table 8, GHRH analogs #1, 2, 3 and 5 exhibit a significantly higher binding affinity than that of hGHRH-(1-29)-NH₂ for its receptor. Moreover, although the relative binding affinity of GHRH analogs #1 and #5 for the human GHRH receptor do not differ significantly from one another, the affinity of GHRH analog #5 is significantly higher than that of #3.

TABLE 8
In vitro relative binding affinity of GHRH analogs in BHK cells expressing the human GHRH receptor.

| | | | | Relative binding affinity (R1) of |
|---|---|---|---|---|
| | | | | compounds in comparison |
| | | | | with hGHRH(1-29)NH₂ in BHK |
| | | IC₅₀ (molar | | cells expressing the |
| No. | Name of compound | concentration) | | hGHRH receptor |
| 1 | [D-Ala2, Ala8, Ala15, | 33 ± 12 | pM | 499 ± 234 |
| | Lys22]human GHRH(1-29)NH2 | | | |
| 2 | [Ala8, Ala9 Ala15, Ala22]human | 0.77 ± 0.09 | nM | 3.70 ± 0.52 |
| | GHRH(1-29)NH2 | | | |
| 3 | [D-Ala2, D-Tyr10, Lys22]human | 6.3 ± 1.1 | pM | 239 ± 55 |
| | GHRH(1-29)NH2 | | | |
| 4 | [D-Ala2, Ala8, D-Tyr10, Ala15, | 37 ± 4 | nM | 0.05 ± 0.01 |
| | D-Lys21, Lys22] human GHRH(1- | | | |
| | 29)NH2 | | | |
| 5 | [D-Ala2, D-Tyr10, D-Ala15, | 6.0 ± 2.4 | pM | 939 ± 249 |
| | Lys22] human GHRH(1-29)NH2 | | | |

TABLE 9
In vitro potency index of GHRH analogs after 60-min incubation in human plasma.

| | | Residual | | | In vitro |
|---|---|---|---|---|---|
| | | peptide | | | potency index |
| No. | Name of compound | concentration* | R1 | R2 | (R1 × R2) |
| 1 | [D-Ala2, Ala8, Ala15, | 79 ± 4 | 499 ± 234 | 1.52 ± 0.18 | 758 |
| | Lys22]human GHRH(1- | | | | |
| | 29)NH2 | | | | |
| 2 | [Ala8, Ala9 Ala15, | Not tested | 3.70 ± 0.52 | Not tested | Not tested |
| | Ala22]human GHRH(1- | | | | |
| | 29)NH2 | | | | |
| 3 | [D-Ala2, D-Tyr10, | 87 ± 5 | 239 ± 55 | 1.69 ± 0.22 | 404 |
| | Lys22]human GHRH(1- | | | | |
| | 29)NH2 | | | | |
| 4 | [D-Ala2, Ala8, D- | Not tested | 0.05 ± 0.01 | Not tested | Not tested |
| | Tyr10, Ala15, D-Lys21, | | | | |
| | Lys22] human | | | | |
| | GHRH(1-29)NH2 | | | | |
| 5 | [D-Ala2, D-Tyr10, D- | 92 ± 5 | 939 ± 249 | 1.78 ± 0.22 | 1671 |
| | Ala15, Lys22] human | | | | |
| | GHRH(1-29)NH2 | | | | |

*% of initial content at time 0; R1: Relative binding affinity of compounds in comparison with hGHRH(1-29)NH2 in BHK cells expressing the hGHRH receptor; R2: Relative resistance to in vitro proteolysis of compounds in comparison with hGHRH(1-29)NH2

As can be seen in Table 9, the in vitro potency index of GHRH analogs #1, 3 and 5 reaches values of 758, 404 and 1671, respectively. In other words, these three (3) analogs have simultaneously a significantly higher binding affinity to their receptor as well as a significantly better resistance to proteolysis upon an in vitro 60-min incubation in human plasma, in comparison with the native hGHRH(1-29)NH₂. Moreover, as can be seen in Table 10 below, the in vitro potency index of GHRH analogs is even higher upon a 180-min incubation in human plasma.

TABLE 10
In vitro potency index of GHRH analogs after 180-min incubation in human plasma.

| | | Residual peptide | | | In vitro potency index |
|---|---|---|---|---|---|
| No. | Name of compound | concentration* | R1 | R2 | (R1 × R2) |
| 1 | [D-Ala2, Ala8, Ala15, | 68 ± 1 | 499 ± 234 | 2.96 ± 0.02 | 1477 |
| | Lys22]human GHRH(1- | | | | |
| | 29)NH2 | | | | |
| 2 | [Ala8, Ala9 Ala15, | Not tested | 3.70 ± 0.52 | Not tested | Not tested |
| | Ala22]human GHRH(1- | | | | |
| | 29)NH2 | | | | |
| 3 | [D-Ala2, D-Tyr10, | 81 ± 1 | 239 ± 55 | 3.54 ± 0.23 | 846 |
| | Lys22]human GHRH(1- | | | | |
| | 29)NH2 | | | | |
| 4 | [D-Ala2, Ala8, D- | Not tested | 0.05 ± 0.01 | Not tested | Not tested |
| | Tyr10, Ala15, D-Lys21, | | | | |
| | Lys22] human GHRH(1- | | | | |
| | 29)NH2 | | | | |
| 5 | [D-Ala2, D-Tyr10, D- | 74 ± 7 | 939 ± 249 | 3.21 ± 0.31 | 3014 |
| | Ala15, Lys22] human | | | | |
| | GHRH(1-29)NH2 | | | | |

*% of initial content at time 0; R1: Relative binding affinity of compounds in comparison with hGHRH(1-29)NH2 in BHK cells expressing the hGHRH receptor ± SEM; R2: Relative resistance to in vitro proteolysis of compounds in comparison with hGHRH(1-29)NH2 ± SEM.

The next step was to test whether the same observations held true after incubation in human serum. Results for GHRH analog #5 can be seen in Table 11. Again, upon 60 or 180 minutes of incubation in human serum, the GHRH analog #5 still presented a significantly higher in vitro potency index, compared to the native hGHRH(1-29)NH₂.

TABLE 11
In vitro potency index of GHRH analog # 5 after
60 and 180-min incubation in human serum.

| | | In vitro | | | |
|---|---|---|---|---|---|
| | | potency | Residual | | In vitro |
| | | index | peptide | | potency index |
| | R2 | (R1 × R2) | concentration | | (R1 × R2) |
| R1 | (60 min) | (60 min) | (180 min)* | R2 (180 min) | (180 min) |
| 939 ± 249 | 1.55 ± 0.04 | 1455 | 62 ± 2 | 2.93 ± 0.87 | 2751 |

*% of initial content at time 0; R1: Relative binding affinity of compounds in comparison with hGHRH(1-29)NH2 in BHK cells expressing the hGHRH receptor ± SEM; R2: Relative resistance to in vitro proteolysis of compounds in comparison with hGHRH(1-29)NH2 ± SEM.

### Example 7

### Use of the GHRH Analog for the Specific Stimulation of In Vivo GH Release

The present invention is directed to the use of the GHRH analog for the specific stimulation of in vivo GH release. Such a use is based upon the following background.

Integration of all the factors that affect GH synthesis and secretion lead to a pulsatile pattern of release, thus a single measurement of plasma GH levels is difficult to interpret. Basal concentrations of GH in blood are very low. In children and young adults, the most intense period of growth hormone release is shortly after the onset of deep sleep. The pattern of GH secretion is episodic, with six to eight pulses per day and very low levels between pulses and is linked to stages 3 and 4 of the sleep cycle, but this association is less evident with increasing age. Some of these pulses are associated with meals, stress, exercise, or slow-wave sleep.

GH pulses occur more frequently and the basal level of plasma GH is higher in females than males who have fewer GH pulses but which are of higher amplitude. In humans there is typically one high secretion pulse and a few lower ones during the 24-h day-night span. Delay, advance or interruption of a sleep phase will shift the main GH secretion pulse correspondingly. At least in humans, GH secretion is also controlled by an endogenous circadian rhythm. When the sleep period is shifted from its normal time, some GH is still secreted during the early night according to the endogenous clock. GH secretion is highest during growing and early adulthood. In humans, the secretion rate starts to decrease during the fourth decade of life. During aging the daytime secretion pulses diminish first, while the sleep-associated GH pulse persists.

In animals, it is more difficult to find a correlation between GH secretion and sleep because many animal species have typically several sleep phases of variable lengths during the 24-h day-night span. However, elevated plasma GH levels during sleep have been demonstrated in several mammals. In the rat, which is a widely used animal model in neuroscience, the GH secretion is pulsatile with an approximately 3.3-h cycle. This rhythm is associated with an ultradian sleep-wake rhythm with the same cycle length, so that the GH pulses precede the sleep maxima by about 24 min. Short-term (3 h) total sleep deprivation during the light phase resulted in a decrease of GH secretion during the deprivation in the rat.

### Experimental Results

### I. Rat Study

Each sample was blind tested in duplicate and the result represents the mathematical mean of two. The source of plasma and samples was unknown to the analyst.

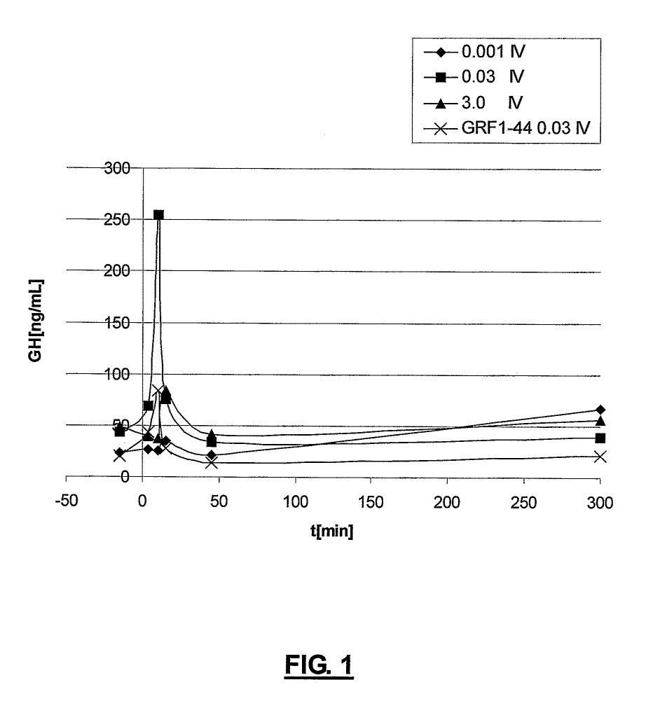

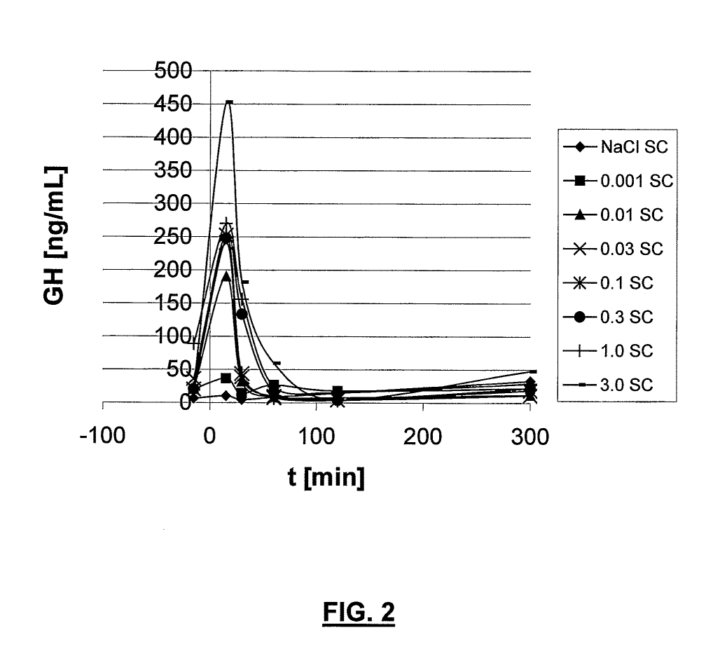

The results of rat plasma testing for rat GH are presented in Table 12 below. Each value in the Table 12 represents the mathematical mean of two animals. The same data were then plotted against time and pharmacodynamic curves are presented in FIG. 1 for the intravenous and in FIG. 2 for the subcutaneous administrations.

Growth hormone areas under the curves (AUC) for different time duration are presented in Table 13.

The data show that both intravenous and subcutaneous administrations of GHRH analogue #5 elicited a dose-dependent response: secretion of GH into peripheral blood. Significant inter-animal variation in GH level was observed. This confirms the observations of others.

Most of the animals exhibited elevated pre-administration concentration of circulating growth hormone. There was a trend for GH concentration to go up again at about 300 minutes (5 hours) post GHRH or NaCl injection in all groups of rats.

TABLE 12
Amplitude of Rat Growth Hormone Secretion, at various
time points, in response to GHRH analogue # 5
administration in adult female rats.

| GHRH | Plasma Rat Growth Hormone (ng/ml) | | | | | | | | |
|---|---|---|---|---|---|---|---|---|---|
| mg/kg | Time post-GHRH administration (minutes) | | | | | | | | |
| BW Route | −120 | 4 | 10 | 15 | 30 | 45 | 60 | 120 | 300 |
| NaCl SC | 6.55 | ND | ND | 10.15 | 4.85 | ND | 8.55 | 14.65 | 32.79 |
| 0.001 SC | 20.15 | ND | ND | 36.7 | 14.2 | ND | 26.85 | 17.8 | 21.85 |
| 0.01 SC | 20.4 | ND | ND | 190.4 | 31.9 | ND | 8.5 | 7.6 | 11.95 |
| 0.03 SC | 36.1 | ND | ND | 240.9 | 39.05 | ND | 9.5 | 4.6 | 11.25 |
| 0.1 SC | 20.4 | ND | ND | 252.4 | 43.8 | ND | 7.65 | 4.45 | 18.7 |
| 0.3 SC | 20.7 | ND | ND | 247.9 | 133.5 | ND | 16.75 | 4.00 | 21.8 |
| 1.00 SC | 88.95 | ND | ND | 270.0 | 155.85 | ND | 24.35 | 15.85 | 28.85 |
| 3.00 SC | 20.05 | ND | ND | 453.0 | 181.55 | ND | 59.4 | 4.45 | 47.9 |
| 0.001 IV | 23.85 | 26.2 | 25.85 | 34.65 | ND | 21.15 | ND | ND | 67.15 |
| 0.03 IV | 43.15 | 68.45 | 254.65 | 75.1 | ND | 33.4 | ND | ND | 38.75 |
| 3.0 IV | 48.6 | 38.7 | 36.95 | 83.65 | ND | 41.6 | ND | ND | 56.7 |
| GHRH (1-44) | 20.2 | 43.7 | 83.9 | 27.9 | ND | 14.1 | ND | ND | 21.7 |
| 0.03 IV | | | | | | | | | |

BW: body weight;
ND: not determined.

As shown in Table 12, Rat Growth Hormone (ng/mL) was measured in duplicate. Values represent the mean of two animals per time point. The Route represents the route of administration which was either subcutaneous (SC) or intravenous (IV).

TABLE 13
Cumulative Rat Growth Hormone Secretion in adult female
rats in response to GHRH analogue # 5 administration,
as determined by GH Area Under the Curve (AUC).

| GHRH mg/kg | GH AUC | | GHRH mg/kg | GH AUC | |
|---|---|---|---|---|---|
| BW SC Route | 120 min | 300 min | BW IV Route | 45 min | 300 min |
| NaCl SC | 1165 | 5434 | 0.001 IV | 1197 | 12280 |
| 0.001 SC | 2914 | 6482 | 0.03 IV | 3407 | 12060 |
| 0.01 SC | 5153 | 6913 | 3.0 IV | 2384 | 14299 |
| 0.03 SC | 6192 | 7618 | GHRH (1-44) | 1380 | 5754 |
| | | | 0.03 IV | | |
| 0.1 SC | 6423 | 8507 | | | |
| 0.3 SC | 8636 | 10958 | | | |
| 1.0 SC | 10425 | 14448 | | | |
| 3.0 SC | 15562 | 20273 | | | |

BW: body weight.

As shown in Table 13, the Route represents the route of administration which is either subcutaneous (SC) or intravenous (IV). Furthermore, GH AUC was determined 45, 120 or 300 minutes post-GHRH administration.

### II. Dog Study

Each sample was blind tested in duplicate and the result represents the mathematical mean of two. The source of plasma and samples was unknown to the analyst.

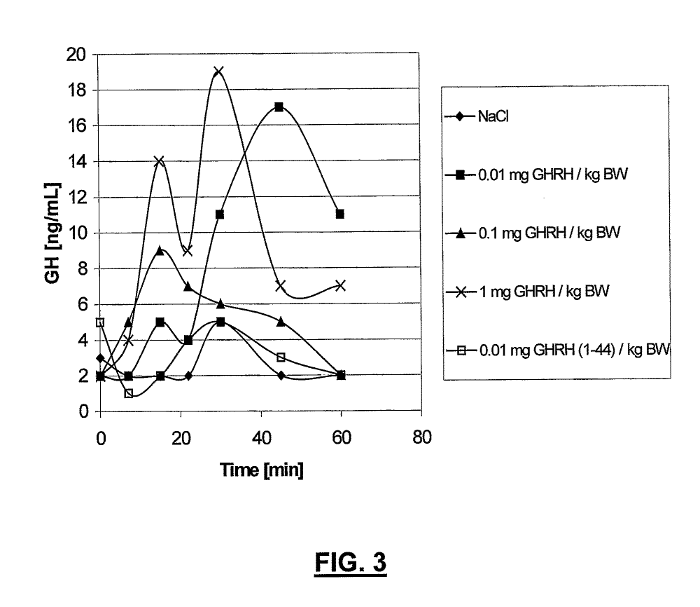

The results of canine plasma testing for canine GH are presented in Table 14 below. The same data were then plotted against time and pharmacodynamic curves are presented in FIG. 3 for the subcutaneous administrations.

The data show that subcutaneous administrations of GHRH analogue #5 elicited a dose-dependent response: secretion of GH into peripheral blood.

There was a trend for GH concentration to go up again at about 30 or 50 minutes post GHRH administration depending on the dose injected.

No treatment-related clinical signs were observed following GHRH analogue administration into both rats and the dog.

TABLE 14
Amplitude of Canine Growth Hormone Secretion,
at various time points, in an 8-mointh-old Beagle dog
in response to GHRH analogue # 5 administration.

| GHRH | Canine Growth Hormone (ng/ml) | | | | | | |
|---|---|---|---|---|---|---|---|
| mg/kg BW | Time post-GHRH administration (minutes) | | | | | | |
| Route | 0 | 7 | 15 | 22 | 30 | 45 | 60 |
| NaCl SC | 3 | 1.99 | 1.99 | 1.99 | 5 | 1.99 | 1.99 |
| 0.01 SC | 1.99 | 1.99 | 5 | 4 | 11 | 17 | 11 |
| 0.1 SC | 1.99 | 5 | 9 | 7 | 6 | 5 | 1.99 |
| 1 SC | 1.99 | 4 | 14 | 9 | 19 | 7 | 7 |
| hGHRH | 5 | 1 | 1.99 | 4 | 5 | 3 | 1.99 |
| (1-44) 0.01 | | | | | | | |
| SC | | | | | | | |

### Data Interpretation

The data presented above clearly demonstrate that the synthetic GHRH analogue #5 recognizes GHRH receptors in both rat and dog pituitary and triggers GH response and secretion into circulation. In a rat, the response is dose-dependent both in terms of height of peak amplitude and AUC for the peak duration. The peak secretion following single subcutaneous injection is between 10-15 minutes and 4-10 minutes following intravenous injection. GH secretion in response to GHRH analogue #5 is twice larger than GH secretion in response to natural hGHRH(1-44)NH₂ both in terms of pulse amplitude and AUC. The highest GHRH analogue #5 single IV dose induced transient somatotroph desensitization.

In the dog, like in the rat, GH secretion in response to GHRH analogue #5 is dose-dependent. The peak secretion following single subcutaneous injection is between 5 and 15 minutes and there clearly is a second GH peak not observed in response to saline or native GHRH indicating longer stability of the analogue in canine plasma. GH response to GHRH analogue #5 is significantly larger than GH secretion in response to natural hGHRH(1-44)NH₂ (AUC not measured).

### CONCLUSIONS

In vivo proof-of-concept has been established. GHRH(1-29)NH₂ synthetic analog of the amino acid sequence of H-Tyr-DAla2-Asp-Ala-Ile-Phe-Thr-Asn-Ser-DTyr10-Arg-Lys-Val-Leu-DAla15-Gln-Leu-Ser-Ala-Arg-Lys-Lys22-Leu-Gln-Asp-Ile Met-Ser-Arg-NH₂ in which Ala2, Tyr10, Gly15, and Leu22 of the first 29 amino acids of natural GHRH have been replaced by D-Ala2, D-Tyr10, D-Ala15, and Lys22 binds to GHRH receptor on somatotrophs in rat and dog pituitaries and stimulates secretion and release of growth hormone in a dose-dependent manner. GHRH analogue #5 is at least two times more potent in vivo than the natural 44 amino acid GHRH.

### Human Clinical Trials (Phase II)

### Growth Hormone

Growth hormone (GH, somatotropin) promotes linear growth in children and is produced throughout life. Pituitary GH secretion is primarily regulated by the interaction of two hypothalamic peptides, growth hormone-releasing hormone (GHRH) and somatostatin. GHRH stimulates the secretion and synthesis of GH; and somatostatin inhibits it. A number of peripheral factors regulate GH secretion; including insulin-like growth factor-1 (IGF-1), which is produced by the liver in response to GH and acts on the hypothalamus to exert a negative feedback on GH secretion. The main effects of GH are stimulation of growth in bone metaphyses during growing, anabolic effect in the muscles, conservation of proteins and carbohydrates, and mobilization of fat for energy sources (lipolysis). The production of GH declines with age. In humans, the production of GH after the age of 21 to 31 falls by about 14% per decade so that the total 24-hour GH production rate is reduced to half by the age of 60. Daily production of GH in 20-year-old humans is about 500 μg, 40-year-olds produce about 200 μg per day, and 80-year-olds, 25 μg per day (2, 3).

### The GHRH(1-29)NH₂ Synthetic Analog

Like naturally occurring GHRH, GHRH(1-29)NH₂ synthetic analog stimulates the pituitary gland to release GH, resulting in an increase in the concentration of GH in the plasma. It has superior combined binding affinity to human GHRH(1-44)NH₂ pituitary receptor and exhibits resistance to proteolysis in human serum and plasma in vitro.

### Nonclinical Studies

The ability of GHRH(1-29)NH₂ synthetic analog to stimulate GH secretion has been studied in vitro and in vivo in rats and dogs.

In vitro studies show GHRH(1-29)NH₂ synthetic analog has 939-times greater binding affinity to the human GHRH receptor than human GHRH(1-29)NH₂ and at least as high as human GHRH(1-44)NH₂. Moreover, after a 420-minute incubation in human plasma, the acetate form of GHRH(1-29)NH₂ synthetic analog was at least 25 times more resistant to proteolysis than human GHRH(1-44)NH₂.

In vivo proof-of-concept has been established. GHRH(1-29)NH₂ synthetic analog binds to GHRH receptors on somatotrophs in rat pituitary and stimulates secretion and release of rat GH dose-dependently. The optimal dose for subcutaneous (sc) administration was found to be between 10 and 30 μg/kg body weight. The same conclusions were drawn from studies in dogs and the optimal sc dose was 10 μg/kg body weight.

In single-dose toxicity studies, the administration of GHRH(1-29)NH₂ synthetic analog to Sprague-Dawley rats at concentrations of 0.001, 0.01, 0.03, 0.1, 0.3, 1 and 3 mg/kg body weight was well tolerated and there were no apparent changes in mortality, clinical signs, body weight, pharmacokinetic and pharmacodynamic assessments in comparison with the positive controls.

In repeat-dose toxicity studies, the administration of GHRH(1-29)NH₂ synthetic analog at dose levels of 0.03, 0.3 and 3 mg/kg to Sprague-Dawley rats for 28 consecutive days was well tolerated. No mortality, adverse clinical sign, effects on body weight, food consumption, opthalmology, haematology, coagulation, clinical chemistry, urinalysis, organ weights, macroscopic or microscopic observations were noted following the dosing or recovery periods. The no adverse effect level for this study was determined to be 3 mg/kg body weight.

The administration of the GHRH(1-29)NH₂ synthetic analog at dose levels of 0.03, 0.3 and 3 mg/kg to Beagle dogs for 28 consecutive days followed by a 28-day recovery period was well tolerated. No mortality, adverse clinical sign, effects on body weight, food consumption, opthalmology, haematology, coagulation, clinical chemistry, urinalysis or organ weights were noted following the dosing or recovery periods. Microscopic findings at the injection sites were noted among treated animals, however, the changes were reversed following the recovery period. A no adverse effect level for this study was determined to be 3 mg/kg.

There was no evidence of mutagenicity in Salmonella strains, Chinese Hamster Ovary cells, or in the erythrocyte micronucleus test. It was concluded that GHRH(1-29)NH₂ synthetic analog is not a clastogenic agent.

### Effects in Humans

GHRH(1-29)NH₂ synthetic analog was tested in Phase I/II study in healthy male volunteers. The study was conducted by CTCRO S.A. Buenos Aires, Argentina.

### Objectives

The primary objectives of the Phase I/II study were to assess safety and tolerability of escalating doses of the GHRH(1-29)NH₂ synthetic analog (Maximal Tolerated Dose—MTD) and to determine the minimal effective dose (MED) of the GHRH(1-29)NH₂ synthetic analog in terms of stimulation of GH and IGF-1 secretion. The secondary objectives were to establish the lowest dose of the GHRH(1-29)NH₂ synthetic analog that induces maximal response with a single subcutaneous administration into healthy male volunteers, to determine the MED for subsequent studies and to establish the most representative blood sampling time for evaluation of GH secretion profile after GHRH(1-29)NH₂ synthetic analog administration to be used in subsequent studies.

### Endpoints

In order to evaluate the safety of the drug, all reported adverse events were considered during the study. The effects of the drug exerted specifically on the liver, renal, thyroid, cardiac and haematological functions were evaluated. The MED was defined as the dose of the GHRH(1-29)NH₂ synthetic analog lower than the subsequent dose that produced the best secretion profile of GH.

### Design

It was a crossover single-blinded Phase I/II trial of escalating doses of the GHRH(1-29)NH₂ synthetic analog controlled versus placebo. Ten healthy middle-aged male volunteers, between 50 and 55 years old, were recruited. All 10 subjects received a single subcutaneous dose of the GHRH(1-29)NH₂ synthetic analog (reconstituted in water for injection), per day for 5 days, in a dose range from 5 to 25 μg/kg of body weight (lower than the maximal dose used to treat GH deficiency in children, which was 30 μg/kg of body weight).

Each volunteer was hospitalized for 6 days. All volunteers were randomized into 5 groups (2 volunteers per group). During the first 24 hours of hospitalization (Day 0), scheduled blood samples were obtained for GH and IGF-1 assessment (GH and IGF-1 baseline sampling) and then from Day 1 until Day 5, following each GHRH(1-29)NH₂ synthetic analog or placebo dose administration, according to the established randomization plan.

On Day 30, an ambulatory control visit took place. Clinical assessments were performed and adverse events were reported. A blood sample was taken to determine the presence of antibodies the GHRH(1-29)NH₂ synthetic analog.

### Procedures

After signing the informed consent form, all volunteers were clinically evaluated by the investigator and a chest x-ray and basal electrocardiogram and routine laboratory tests were performed. The analysis included: haemogram (hematocrit, haemoglobin, complete blood cell count, mean cell volume (MCV), mean corpuscular haemoglobin concentration (MCHC) and platelet count), liver function tests (aminotransferases, total proteins, albumin, alkaline phosphatase, billirubin, lactate dehydrogenase and prothrombin time), biochemistry (creatinine, urea, sodium, potassium, calcium, phosphorus, fasting glucose and postprandial glucose), urinalysis (pH, glucose, blood, proteins, and leukocytes), triiodothyronine, thyroxin and thyroid stimulating hormone (TSH), serology, basal level of IGF-1 and urine drug abuse screening.

Once the volunteers were considered eligible, they were hospitalized for a period of 5 consecutive days. On Day 0 of admission successive samples of blood were taken to perform the basal secretion profile of GH during 24 hours. During 5-day hospitalization period, after the initial blood sampling to determine fasting glucose level, the appropriate dose of the GHRH(1-29)NH₂ synthetic analog or placebo was administered and multiple samplings were performed to assess the profile of GH, postprandial glucose level and electrocardiogram after 60 minutes of the application of the GHRH(1-29)NH₂ synthetic analog/placebo. Eight hours after drug administration, samples were obtained to determine IGF-1 level, thyroid hormones and routine analysis. After 30 days of admission, all the volunteers returned for new clinical evaluation to record any adverse events that could have occurred from the last day of hospitalization period and to take a blood sample for later determination of anti-GHRH(1-29)NH₂ synthetic analog antibodies.

Serum human GH was measured by using a commercial DSL-1900 ACTIVE® Human Growth Hormone Coated-Tube Immunoradiometric Assay Kit purchased from Diagnostic Systems Laboratories, Inc., Texas, USA. Serum IGF-1 was measured using DSL-9400 ACTIVE® Free Insulin-Like Growth Factor-I Coated-Tube IRMA Kit purchased from Diagnostic Systems Laboratories.

Human anti-GHRH antibodies were measured in ELISA developed LAB Pre-Clinical Research Intl.

### Results

### Safety

During the conduct of the study, no serious adverse events were reported. Two volunteers reported “mouth dryness”, and the other two reported thirst with the different doses used during the hospitalization period, even when they received placebo. Two stated headache when they received 15 μg/kg of GHRH analog and one presented arterial hypotension associated with orthostatism when he received 10 μg/kg. Only one volunteer reported cough when he received 10 μg/kg and one reported itching the day that he received placebo. All the mentioned events were considered mild, except for one episode of headache, which was considered moderate and required one dose of paracetamol as symptomatic treatment.

Regarding the laboratory anomalies found in the control assessments, it is important to observe that all the volunteers presented anaemia throughout the study, with a mean decrease of haematocrit of 10.1±1.9 (7 to 14 points), and 2.8±0.8 g/dl of haemoglobin (from 2 to 4 g/dl). All the cases were considered possibly related to the study drug because of its temporal relation, although the aetiology of this adverse event was related to multiple blood sampling performed throughout the whole study. Since the haemoglobin was never less than 10 g/dl, all the cases were considered as adverse events.

Other haematological abnormalities were judged mild and transitory. Those anomalies were:
leukocytosis with eosinophilia in one volunteer (12,400 white blood cells (WBC)/mm3 and 808 eosinophyls/mm3 respectively), reported on Day 5 and corresponding to 5 μg/kg dose and considered as probably related to the drug by the investigator;
leukopenia with neutropenia (3,800 WBC/mm3 and 1,406 neutrophyles/mm3, respectively) reported on Day 2, after receiving 10 μg/kg-dose, lasting only one day and considered as possibly related because its temporal relationship with the drug;
relative lymphocitosis, only 1 case, reported on Day 1, with 4 days of duration and considered probably related to the drug.

Two volunteers presented postprandial hyperglycemia. In one of them it was a transitory episode, lasting only one day after receiving 15 μg/kg of the GHRH(1-29)NH₂ synthetic analog. The second one persisted for 5 days, after 10, 15, 25 μg/kg doses and even following the placebo administration. The first case was considered as possibly related to the drug because of its temporal relationship, whereas the second case was considered definitively related to the drug because of its duration.

One case of aminotranspherase elevation, considered mild (less than 2.5 times the upper normal limit), was reported from Day 1 to Day 5 and that was considered probably related to the study drug by the investigator.

Neither thyroid anomalies nor cardiac dysfunction were reported. None of the adverse events reported presented any clinical repercussion and none of them received specific treatment.

None of the subject samples tested had detectable antibodies to the GHRH(1-29)NH₂ synthetic analog at the dilution tested.

### Efficacy

To determine MED, GH secretion curves were drawn and the area under the curve (AUC) calculated for each study drug dose.

GH levels were unequivocally higher at any dose of the GHRH(1-29)NH₂ synthetic analog administered when compared to the placebo. GH secretion peak was reached between 8 and 180 minutes after drug administration and the effect was relevant during the first 12 hours after the application, without significant modifications of the nocturnal GH secretion, with the exception of the highest dose.

The results obtained during this trial in a total of 10 volunteers indicate that doses of the GHRH(1-29)NH₂ synthetic analog greater than 10-15 μg/kg body weight do not seem to increase significantly the study drug efficacy.

The data clearly show a significant and dose-dependent effect of subcutaneous administration of the GHRH(1-29)NH₂ synthetic analogue on GH release, at least during the first 4 hours of high-frequency sampling immediately after injection. Outside this period, GH secretion levels were either very low or undetectable.

Baseline GH secretion in all volunteers showed only one peak of GH in the nocturnal period, between 720 and 960 minutes. A similar pattern was observed with the administration of placebo, with the exception of 3 subjects that had nocturnal peak later at night.

For all doses administered, a second small peak between 750 and 960 minutes, of variable intensity in different volunteers, was also observed.

The AUC of GH levels secreted during 24 hours after the GHRH(1-29)NH₂ synthetic analog administration were calculated. Although the median of GH levels was increasing progressively with the increasing dose of the GHRH(1-29)NH₂ synthetic analog, from 3.75 to 180.08 ng/ml (ranges 0-348 ng/ml and 59.02-641.69 ng/ml, respectively) the only significant difference found was when the AUC reached after placebo administration was compared to the AUC obtained after 25 μg/kg-dose administration (p<0.03).

When the AUCs of the GH level obtained in the first 12 hours after polypeptide administration (diurnal hours) were compared, a significant difference was found between the mean values reached with different doses of the GHRH(1-29)NH₂ synthetic analog. When compared to the effect of placebo, the AUC was always higher for the GHRH(1-29)NH₂ synthetic analog administration, independently of the dose administered (0.00-61.76-71.25-135.17, and 149.48 for placebo, 5, 10 15 and 25 μg/kg body weight of the GHRH(1-29)NH₂ synthetic analog respectively; p<0.005). The difference between the AUC obtained at 5 and 10 μg/kg was also significant (61.76 vs. 71.25; p<0.04), although there was no significant difference between doses of 10 and 25 μg/kg, 10 and 15 μg/kg and 15 and 20 μg/kg body weight. Different results were observed during the 12 nocturnal hours, where, as in the 24-hour analysis, the only significant difference was between the 25 μg/kg dose and placebo, without differences between the other doses. IGF-1 plasma levels were obtained after each GHRH(1-29)NH₂ synthetic analog administration. Plasma IGF-I levels reflect the plasma GH AUC of the preceding 48 hours. When the results were expressed as dose response curves for each volunteer, no significant differences were found between the doses.

But since the different doses were not administered in the escalating sequence nor were they injected in the same order to different volunteers, it was decided that it would be more appropriate to express the IGF-1 levels reached by each subject in terms of the day of treatment, rather than the dose received. IGF-1 mean levels increased progressively from Day 1 of treatment to Day 5, reaching a significant difference between them (156 ng/ml on Day 1 versus 231 ng/ml on Day 5; p<0.005).

### SUMMARY

Within the dose range used in this study, the GHRH(1-29)NH₂ synthetic analog seems to be a safe drug, since it has caused neither unexpected adverse events nor severe adverse events, and an effective drug since it produced a significant increase in GH and IGF-1 levels when administered subcutaneously once a day for more than one day. According to these data, it would be possible to assume that the dose to use in the next studies could be from 10 to 15 μg/kg body weight, since upon administration of higher doses there seem to be no further significant increase in efficacy, measured by GH levels, and thus possible toxicity associated with greater doses could be avoided.

Clinical Experience with GHRH

There is a great deal of experience with recombinant human growth hormone and synthetic GHRH particularly in children. In adults with GH-deficiency, short-term (12 to 24-hour) continuous intravenous infusions of GHRH to normal adult men increased episodic nocturnal GH secretion proportionately to the GHRH dose, with preservation of the GH response to bolus intravenous GHRH testing. In a study of five adult men treated with continuous intravenous infusions of GHRH for 14 days, significant increases in IGF-I levels, augmentation of pulsatile GH release, and preservation of the response to supramaximal bolus doses of GHRH were found. These findings, taken together, suggest that the normal pattern of endogenous GH pulsatile secretion is preserved and enhanced during GHRH administration, and that there is no evidence of significant in vivo somatotrope desensitisation by constant exposure to GHRH in humans. Alternate day intravenous injections of GHRH for 12 days restored the acute GH responses to GHRH of older men to levels comparable with those of young men. Similar acute GH responses, accompanied by a significant increase in IGF-I levels, also occurred after administration of intravenous GHRH for 8 days to postmenopausal women. More recently, administration of GHRH to healthy old men for 14 days was found to reverse age-related reductions in circulating levels of GH and IGF-1, with preservation of diurnal GH secretory patterns, and without adverse effects or changes in blood pressure, serum glucose, or urinary C-peptide.

Taken together, the above observations suggest that GHRH administration may represent an alternative, and perhaps physiological, method of increasing subnormal GH and IGF-1 levels in healthy old men and women.

In the limited number of studies that have been conducted, it appears that GHRH has fewer, and less severe side effects than recombinant human GH replacement therapy. Serum antibodies to GHRH develop in most children but their significance on the effectiveness of longterm GHRH treatment is unknown. These antibodies are not neutralizing. Extending the half-life of GHRH in the circulatory system, and increasing its binding affinity to the GHRH receptor are currently actively researched.

### Rationale for the Phase II Study

Chronic kidney disease (CKD) is a common disorder causing an enormous economic burden to health systems worldwide. Despite advances in renal replacement technologies and treatment costs of $40-50,000 per year, patient morbidity on dialysis is excessive and annual mortality rates remain around 15-25% in registries around the globe. Hence, the prognosis of end-stage renal disease (ESRD) is comparable with highly malignant neoplastic disease.

Most kidney disorders have a tendency to progress gradually to ESRD by an autodestructive process that is characterized by glomerular hyperfiltration and sclerosis, and tubulointerstitial inflammation and fibrosis. In recent years, pharmacological renoprotective concepts have been developed that involve inhibition of the renin-angiotensin system and strict blood pressure control. By this approach, the process of progression to ESRD can be slowed but not completely arrested. It is expected that an increasing proportion of patients will be in advanced but not end-stage CKD in the future.

CKD is frequently associated with a catabolic state of metabolism characterized by anorexia, increased proteolysis and subclinical inflammation. Renal replacement therapy is usually initiated early in patients with this malnutrition-inflammation syndrome, and their morbidity and mortality on dialysis is particularly high. Successful reversal of uremic catabolism in advanced pre-dialytic CKD could help to improve patient morbidity and survival in this population and even to postpone the need for dialysis in many patients.

While the aetiology of uremic cachexia is multifactorial, a mechanism of potential therapeutic interest is the endogenous GH resistance in renal failure. In uremia, the biological actions of this key anabolic hormone are hampered by multiple mechanisms including reduced GH receptor expression, impaired post-receptor signaling, increased plasma protein binding of the GH-induced mediator protein IGF-1 and impaired IGF-1 tissue signaling. Endogenous GH release is variable but usually insufficient to compensate for the target tissue resistance. It has been proposed that this state of endogenous GH resistance might be overcome by administration of GH administered at pharmacological doses.

Indeed, GH treatment improves growth in children with predialytic or end-stage CKD and increases lean body mass in adult patients even in ESRD. Notably, the clinical response to exogenous GH appears to depend on the degree of renal failure, being least marked in ESRD patients. An additional argument for this approach is the finding from short-term trials as well as from long-term surveillance studies that GH has a small but consistent stimulating effect on glomerular filtration rate (GFR, the level of renal function) mediated by an IGF-1 dependent mechanism. This effect may contribute to a delay for the need for renal replacement therapy.

The experience with GHRH in the CKD population is very limited to date. The pituitary response to GHRH appears to be intact even in end-stage CKD. Theoretically, the clinical effects of GHRH may be inferior to those of direct high-dose GH treatment in this partly GH-resistant population. This may limit the indication of GHRH analogues to the pre-endstage CKD population, where GH resistance is less marked. Indeed, in a small study of GHRH(1-29) in children with different stages of CKD, a very good growth response in 3 out of 3 pre-endstage patients was demonstrated, whereas 2 out of 3 dialysis patients were nonresponders.

Since progression of CKD to end-stage tends to be slow and may even be slowed further by stimulation of the GH-IGF-1 system, many patients may qualify for long-term GHRH analogue treatment. This will make cumulative treatment costs an issue for regulatory authorities. It appears that GHRH analogues can be produced at lower costs than recombinant human GH. Hence, under cost-efficacy considerations, GHRH analogues may turn out to be preferable to recombinant GH in this population.

Taking all of these arguments together, GHRH analog therapy, if effective, may be a very attractive adjunctive therapy in patients with advanced pre-endstage CKD. A clinical trial is, therefore, proposed to determine the efficacy the GHRH(1-29)NH₂ synthetic analog in stimulating GH secretion and reversing uremic catabolism in adult patients in whom renal function is reduced to 10-20% of normal and who exhibit mild, moderate or severe malnutrition. Since the study period will be 4 weeks, sensitive outcome measures will be used. Dual X ray absorptiometry (DEXA) will be used to measure changes in body composition, and isotope-labeled leucine kinetics will be used to assess protein balance before and after 4 weeks of twice daily sc administration of the GHRH(1-29)NH₂ synthetic analog.

Phase II, Double Blind, Placebo Controlled, Parallel Group, Multi-Centre Study of Twice Daily Dosing with the GHRH(1-29)NH₂ Synthetic Analog 1 mg Per Dose Sc for 28 Days, in Malnourished Patients with Chronic Kidney Disease

### Primary Objective

To determine the effect of treatment on protein turnover as assessed by ¹³C-leucine kinetics.

### Secondary Objective

To determine the effects of the GHRH(1-29)NH₂ synthetic analog on:
1. Endogenous time-integrated 24-hour GH secretion and circulating free IGF-1.
2. Fat-free mass and fat mass as assessed by DEXA, bioimpedance and conventional anthropometry (skinfolds/arm circumference).
3. Biochemical parameters of nutritional and metabolic state (serum albumin, prealbumin, transferrin, leptin, adiponectin, glycosylated haemoglobin (HbA_{1c}), fasting glucose, insulin, lipids).
4. Spontaneous nutrient intake (3-day dietary protocols).
5. Safety and tolerability in subjects with CKD.

### Overall Study Design and Plan-Description

This was a randomized, double-blind, placebo-controlled, parallel-group, multi-centre study of the GHRH(1-29)NH₂ synthetic analog in malnourished subjects with CKD. Twenty-six subjects were required to complete the study.

### Screening—Visit 1, Day-7 to 1

Subjects were provided with written information about the study in advance of the screening visit and were given at least 24 hours to consider the information, and the opportunity to discuss the study with the investigator and to ask questions. The screening visit was rescheduled if the subject required more time to consider participating in the study. Subjects who attend the screening visit were assigned a screening number.

Subjects who had given written informed consent were evaluated at the screening visit for the study entry criteria, including the following:
Demographic details.
Medical history, including concomitant medications.
Subjective Global Assessment (SGA).
Blood tests (haematology, clinical chemistry, blood gas analysis, albumin, HbA1c, thyroid stimulating hormone (TSH), free thyroxin and CRP in case of the presence of the active systemic inflammatory or infectious disease).
Height and weight.
Pregnancy test for women of childbearing potential.

Subjects who were eligible for the study were asked to return to the clinic within 7 days for the baseline visit. Subjects were asked to keep a food diary for 3 days from Day-3 to Day 0. In the case of subjects for whom the screening data was available within 7 days of the visit, the screening and baseline visits was combined.

### Baseline—Visit 2, Days 0 and 1

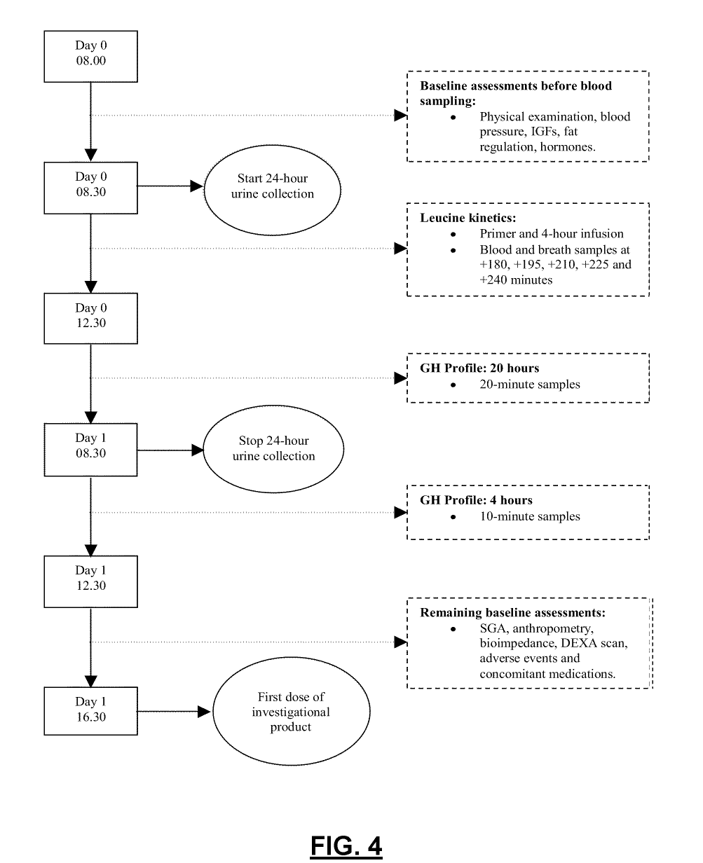

Subjects were admitted to the clinic within 7 days of the screening visit for the baseline assessments. A flow chart of the procedure is shown in FIG. 4. Note that the times (24-hour clock) used in FIG. 4 were for guidance and illustration. A window of ±1 hour was permitted for the start of all procedures. The assessments on Days 28/29 and 0/1 had to be performed at the same times of day. Subjects fasted overnight before their attendance at the clinic and returned the 3-day food diary. Eligible subjects were assigned a randomisation number and were randomised to 28 days treatment with the GHRH(1-29)NH₂ synthetic analog sc or placebo sc. The following assessments were performed prior to administering the first dose of investigational product:
Dietary assessment.
Changes in concomitant medications since screening (if relevant).
Physical examination, including blood pressure and pulse.
Blood tests (haematology including differential white cell count, clinical chemistry including blood gas analysis, plasma proteins, i.e. albumin, pre-albumin and transferrin, fasting insulin, glucose, triglycerides and cholesterol, HADA).
Blood sample for pre-dose endocrine tests (IGF-1, fat regulation and hormones).
24-hour urine collection (mean of creatinine and urea clearances, proteinuria, protein equivalent of nitrogen appearance (PNA), sodium excretion and electrolytes (sodium, potassium, chloride, calcium and phosphate).
Leucine kinetic study.
24-hour GH profile (samples for the first 20 hours taken at 20-minute intervals then samples for the last 4 hours taken at 10-minute intervals).
SGA.
Anthropometry and bioimpedance.
DEXA scan.

Subjects remained fasted until the end of the leucine kinetics study, after which they were given a standard light meal immediately, and at appropriate intervals throughout the 2 study days. The study team decided on the content and timing of meals. Subjects were permitted to drink fluids without sugar, freely, throughout the study days.

At the end of the baseline assessments, at approximately 4.30 (±60 minutes) in the afternoon, the subject received the first dose of study medication; 1.0 mg of the GHRH(1-29)NH₂ synthetic analog or Placebo. Adverse events (AEs) and concomitant medications were recorded after which the subjects were permitted to go home. Doses of study medication were given twice daily at 8.30 am (±60 minutes) and at 4.30 pm (±60 minutes) throughout the 28-day study period.

### First Interim Visit—Visit 3, Day 7

Subjects attended the clinic 7 (±1) days after the first dose of investigational product, before the morning dose and after an overnight fast, for the following assessments:
Changes in concomitant medications.
AEs.
Physical examination, including blood pressure and pulse.
Anthropometry and bioimpedance.
Blood tests (clinical chemistry, transferrin, fasting insulin, glucose, triglycerides and cholesterol).
Endocrine tests (insulin-like growth factors and fat regulation).
Morning dose of investigational product.

### Second Interim Visit-Visit 4, Day 14

Subjects attended the clinic 14 (±1) days after the first dose, before the morning dose and after an overnight fast, for the following assessments:
Changes in concomitant medications.
AEs.
Physical examination, including blood pressure and pulse.
Anthropometry and bioimpedance.
Blood tests (haematology, clinical chemistry including blood gas analysis, pre-albumin, transferrin, fasting insulin, glucose, triglycerides and cholesterol).
Endocrine tests (insulin-like growth factors and fat regulation).
Morning dose of investigational product.

### Third Interim Visit—Visit 5, Day 21

Subjects attended the clinic 21 (±1) days after the first dose, before the morning dose and after an overnight fast, for the following assessments:
Changes in concomitant medications.
AEs.
Physical examination, including blood pressure and pulse.
Anthropometry and bioimpedance.
Blood tests (clinical chemistry).
Endocrine tests (insulin-like growth factors and fat regulation).
Morning dose of investigational product.
Subjects were reminded to start a 3-day food diary on Day 25.

### Final Study Visit—Visit 6, Days 28 and 29

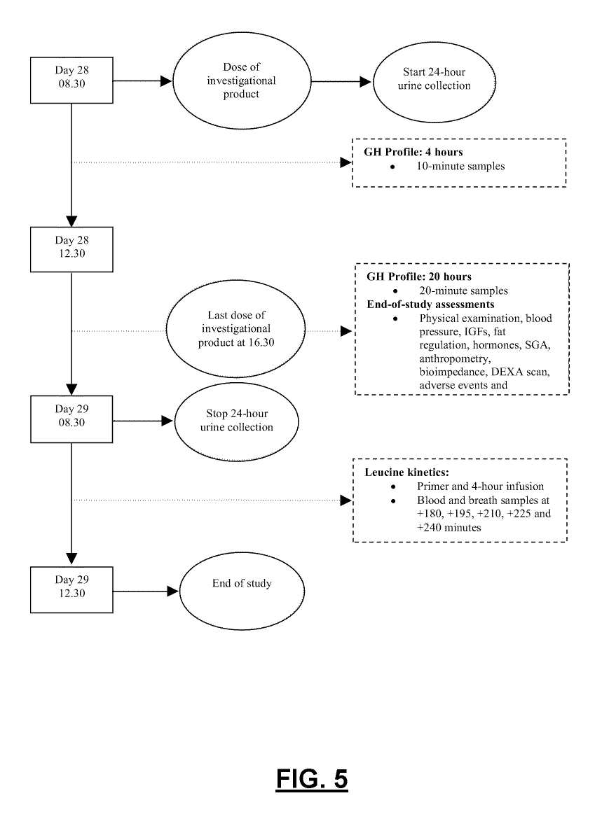

Subjects were admitted to the clinic 28 (±1) days after the baseline visit, before the morning dose and after an overnight fast, for the following assessments. A flow chart of the procedure is shown in FIG. 5. Note that the times (24-hour clock) used in FIG. 5 were for guidance and illustration. A window of ±1 hour was permitted for the start of all procedures. The assessments on Days 28/29 and 0/1 had to be performed at the same times of day.
Final 2 doses of investigational product.
24-hour GH profile (samples for the first 4 hours taken at 10-minute intervals then samples for the last 20 hours taken at 20-minute intervals).
Blood sample for endocrine tests (IGF-1, fat regulation and hormones).
24-hour urine collection (mean of creatinine and urea clearances, proteinuria, PNA, sodium excretion and electrolytes).
Dietary assessment (collection of 3-day food diary).
SGA.
Changes in concomitant medications.
AEs.
Physical examination, including blood pressure and pulse.
Anthropometry and bioimpedance.
DEXA scan.
Blood tests (haematology including differential white cell count, clinical chemistry including blood gas analysis, plasma proteins i.e. albumin, pre-albumin and transferrin, HbA_{1c}, fasting insulin, glucose, triglycerides and cholesterol, HADA).
Leucine kinetic study.

Subjects were permitted to drink fluids without sugar, freely, throughout the study days. The study team decided on the content and timing of meals. Subjects were fasted overnight between Day 28 and Day 29 and remained fasted until the end of the leucine kinetics study, after which they were given a standard light meal immediately. At the end of the leucine kinetic study, the subjects were permitted to go home.

### Follow-Up Visit—Visit 7, Day 42

At 42 (±3) days after the baseline visit, subjects attended the clinic for a follow-up assessment, including:
Physical examination, including blood pressure and pulse.
Anthropometry and bioimpedance.
Changes in concomitant medications.
AEs.
Blood tests (haematology including differential white cell count, clinical chemistry including blood gas analysis, albumin, fasting insulin, glucose, triglycerides and cholesterol, HADA).
IGF-1.
SGA.

### Discussion of Study Design, Including the Choice of Control Groups

CKD is a progressive disorder. Therefore, although suitable subjects for this study were eligible only if they had relatively stable disease, a parallel-group study was appropriate to ensure that the disease did not progress significantly between a first and second baseline, as would be included in the design of a crossover study. Furthermore, if the treatment was effective, the benefit of treatment had to be measurable against a background of worsening disease. Consequently, a relatively short study period was appropriate.

Blood samples for assessment of GH secretion had to be taken over 24 hours because the release of endogenous GH was pulsatile and not consistent throughout the day. Much of endogenous GH release is closely coupled to periods of slow-wave sleep, and the bulk of GH secretion usually occurs between midnight and 4 am. However, Phase I/II data with the GHRH(1-29)NH₂ synthetic analog clearly shows an immediate effect on GH release during the first 4 hours after the GHRH(1-29)NH₂ synthetic analog administration. To document clearly the effect of the GHRH(1-29)NH₂ synthetic analog on endogenous GH secretion, the following protocol had been designed: first, baseline endogenous GH secretion was assessed on Day 0 by sampling blood every 20 minutes for 20 hours and every 10 minutes for 4 hours. At the end of this sampling period (in the afternoon of Day 1), the first injection of the GHRH(1-29)NH₂ synthetic analog was given. At the end of the 4-week treatment period (Day 28), GH secretion was reassessed by another 24-hour secretion profile. This profile started at 8:30 am with the morning injection of the GHRH(1-29)NH₂ synthetic analog. Blood was collected every 10 minutes for the first 4 hours, followed by sampling every 20 minutes for the next 20 hours; this period included the afternoon injection of the GHRH(1-29)NH₂ synthetic analog. In this way, it was possible to assess the evolution of both the pituitary's immediate GHRH response and that of time-integrated 24-hour GH secretion over extended treatment.

Conventional anthropometric measurements, bioimpedance and even body composition by DEXA scan may not have shown a change within the 4-week study period. However, leucine kinetics had been demonstrated to provide an elegant and sensitive marker for protein metabolism.

### Selection of Study Population

This clinical trial was coordinated by the Department of Nephrology, Endocrinology and Metabolic Diseases, Silesian University School of Medicine, Katowice, Poland. Possible recruitment procedures included the following:
Subjects identified from the clinic and outpatient department records were contacted directly by phone by the investigator or his delegate to inquire about their willingness to participate in the study.
Advertisements on bulletin boards.
Subjects were informed about the study during their hospitalisation or normal visit to the outpatient department.

Doctors from other nephrological clinics, hospitals and outpatient departments were asked to inform potential subjects about the study.

### Inclusion Criteria

Subjects who fulfilled all of the following criteria were suitable for inclusion in the study:
1. Male or female above age 40.
2. CKD stage 1V or V defined by a current glomerular filtration rate (GFR) of 10-30 mL/min/1.73 m²).
3. Malnutrition of any severity, defined by serum albumin <40 g/L or body mass index (BMI)<23 mg/m² or 5% loss of body weight in the previous 6 months (protocol amendments 3 and 4, Appendix 16.1.1)
4. Stable clinical condition.
5. Subjects on erythropoietin therapy had to have been on stable treatment for 8 weeks prior to study entry and anticipated to continue throughout the study.
6. Written, informed consent provided.

### Exclusion Criteria

Subjects who fulfilled any of the following criteria were not eligible for the study:
1. Severe fluid overload, as identified by the investigator.
2. Severe renal anaemia, defined by haemoglobin <10 g/dL.
3. Uncontrolled metabolic acidosis, defined as venous HCO₃<22 mmol/L.
4. Rapidly progressive renal failure (GFR loss >20 mL/min/1.73 m² in the previous 12 months).
5. Current or previous history of neoplasia, including GH-producing adenomas.
6. Glucocorticoid treatment within 4 weeks of study Day 1, or subjects who were anticipated to require glucocorticoid treatment. Androgens or oestrogen treatment (including hormonal contraception) was allowed provided the dose was stable for 4 weeks before the screening visit and throughout the entire study.
7. Active systemic inflammatory or infectious disease, including lupus erythematosus and other vasculitis, and human immunodeficiency virus, with CRP level above 10 mg/L.
8. Congestive heart failure.
9. Active liver disease or clinically relevant abnormal laboratory results of alanine aminotransferase (ALT), aspartate aminotransferase (AST), gamma-glutamyl transferase (GGT) that, in the opinion of the investigator, might conflict with the objectives of this trial.
10. Hypothyroidism i.e. elevated TSH and/or low free thyroxin, and patients taking thyroxin supplements.
11. History of drug abuse.
12. Pregnant or lactating women.
13. Subjects taking centrally acting drugs e.g. clonidine, dopamine, some antidepressants (according to the opinion of the investigator).
   Removal of Subjects from Therapy or Assessment

Every subject (or his or her legal representative or proxy consenter, if applicable) had the right to refuse to participate further in the study at any time and without providing reasons. A subject's participation was terminated immediately upon his or her request. The investigator attempted to find out the reason and record this on the CRF. If, at the time of refusal, a dose of the investigational product had already been administered, the subject was advised to agree to follow-up safety investigations (follow-up visit assessments).

The termination of an individual's participation in the study was to be considered in the case of a serious adverse event (SAE) or considerable worsening of the subject's clinical symptoms.

The subject could be withdrawn from the study at any time at the discretion of the investigator, and the reason documented fully on the CRF. If a subject developed any condition that would have excluded him or her from participating in the study that represented a safety concern, he or she was withdrawn immediately. In other cases the investigator decided whether or not to withdraw the subject if there was a conflict with study objectives. The reasons were fully documented on the CRF.

If a subject was discontinued from the study for any reason, he or she was seen regularly as considered clinically appropriate. If the discontinuation was caused by an AE, the subject was seen regularly until the symptoms had disappeared, or were under control, or until suitable treatment had been undertaken.

At the discretion of the Sponsor's Medical Advisor, the study could be cancelled for medical reasons. In addition, the Sponsor retained the right to end the study at any time if the study could not be carried out as agreed upon in the protocol.

### Treatments

### Treatments Administered

All study subjects were randomised in equal numbers to receive active GHRH(1-29)NH₂ synthetic analog or an equivalent volume of placebo, by sc injection, twice daily for 28 days.

Vials of investigational product contained 1.4 mg of the GHRH(1-29)NH₂ synthetic analog. This was reconstituted with 0.7 mL of water for injection and 0.5 mL of the resultant solution (1.0 mg of active substance) was administered, i.e. approximately 15 μg/kg. The diluent, water for injections, was obtained from commercial stock and labelled accordingly.

The first dose of investigational product was administered after completion of the baseline (Day 0) assessments and before the post-dosing assessments. The first day of dosing was designated study Day 1 (Visit 2). A member of the study staff administered all doses.

### Identity of Investigational Product(s)

The GHRH(1-29)NH₂ synthetic analog is an analogue of human peptide sermorelin. It is the acetate salt of an amidated, synthetic 29-amino acid peptide that corresponds to the amino-terminal segment of the naturally occurring GHRH that consists of 44 amino acid residues.

The GHRH(1-29)NH₂ synthetic analog is a white or off-white lyophilised powder and the molecular formula is C₁₅₀H₂₄₉N₄₅O₄₂S₁.

The drug substance was manufactured by PolyPeptide Laboratories Inc, USA, under Good Manufacturing Practice conditions, and a manufacturing license issued by the Food and Drug Administration. The manufacturer had released a statement that GHRH was not of animal origin and thus was not a risk source for Transmissible Spongiform Encephalopathy-prion contamination.

Active investigational product was provided as powder in single-use vials containing 1.4 mg of the GHRH(1-29)NH₂ synthetic analog, 3.8 mg citric acid anhydrate, 8.8 mg sodium citrate dihydrate, and 50 mg mannitol. The contents of the vial were reconstituted with 0.7 mL water for injection.

Placebo was provided as powder in single-use vials containing 3.8 mg citric acid anhydrate, 8.8 mg sodium citrate dihydrate, and 50 mg mannitol. The contents of the vial were reconstituted with 0.7 mL water for injection.

Vials of the GHRH(1-29)NH₂ synthetic analog and placebo were manufactured according to Good Manufacturing Practice standards and supplied by MR Pharma. The diluent, water for injections, was supplied with the investigational product and was obtained from commercial stock. Unused vials were stored at 2-8° C.

Reconstituted solution was stable for 5 days but was used within 24 hours to minimise the risk of bacterial contamination. Vials of reconstituted solution were stored in a refrigerator (at 2-8° C.). Each vial was used once.

### Method of Assigning Subjects to Treatment Groups

At the baseline visit (Day 0), after confirmation that the subject met all of the eligibility criteria for the study, the subject was assigned a randomization number. A statistician created a SAS program that generated the randomization codes for each group, assigning subjects at random to 1 of the 2 treatment arms. An independent person, who was not involved in the study, conducted the final run of the randomization program with a different seed number. This person ensured that the blind was maintained for all involved in the study conduct. Supplies were prepacked and numbered serially so that randomisation was achieved by assigning the subject to the next available number in the sequence. A randomization number was not re-used once it had been assigned to a subject.

### Selection of Doses in the Study

The dose of investigational product, approximately 15 μg/kg, was chosen on the basis of non-clinical studies and preliminary data from a pilot study in humans.

### Selection and Timing of Dose for Each Subject

All subjects received 0.5 mL of the GHRH(1-29)NH₂ synthetic analog or placebo, by sc injection, twice daily for 28 days: at 8:30 am (7:30-9:30) and 16:30 pm (15:30-17:30). This was based on the published data that the most optimal time for GH induction was at the time of low endogenous GH secretion and not at its highest secretion at night.

### Blinding and Code Breaking

The GHRH(1-29)NH₂ synthetic analog and placebo was supplied in identical single-use vials inside boxes containing sufficient supplies for each subject. The labelling of active and placebo investigational product was identical. The language of the original labels was Polish.

Individual code-break envelopes were produced by the independent person at Encorium Ltd who was also responsible for the final creation of the randomization code. This person supplied the code-break envelopes to the center and to the Medical Adviser. The randomization code-break envelopes were opened only in the case of a medical emergency where knowledge of treatment allocation was essential for the management of the subject's condition. If any code-break envelope was opened, the person who opened it, signed and dated the envelope, and gave the reason for opening it. None of the people directly involved in the conduct of the study had access to the treatment codes.

### Prior and Concomitant Therapy

Large doses of glucocorticoid corticosteroids can inhibit the growth stimulating effects of GH. Therefore, subjects were not allowed to take glucocorticoid therapy from 4 weeks before the first dose of investigational product until the end of the study because concomitant treatment might have interfered with the efficacy evaluations.

Subjects who had been treated with erythropoietin could continue treatment provided that the dose in use was stable for 8 weeks prior to the study and was anticipated to continue at the same dose throughout the study period.

Centrally acting drugs e.g. clonidine, dopamine, some antidepressants (according to the opinion of the investigator) were disallowed throughout the study.

### Treatment Compliance

The investigator or pharmacist, according to the local agreement, confirmed receipt of the investigational product in writing, and used the investigational product only within the framework of this clinical study and in accordance with this study protocol. He or she kept a record of the investigational product dispensed, on the Per Patient Drug Accountability Log. The investigational product was stored, when at the site, in a locked storage facility and protected from unauthorised access.

Any unused, partially used or empty containers of the investigational product were returned to the Sponsor, at the latest at the termination of the study. The residual contents of ampoules or vials were disposed of at the study site. Receipt, distribution and return of the investigational product were documented on the forms provided by the Sponsor, giving the following information: study number, sender, receiver, date, mode of transport, type of unit, batch number and expiry date, if applicable.

### Leucine Kinetic Study

Leucine kinetics was measured on Day 0 and Day 29 by a primed-constant infusion technique during substrate and isotopic steady state. The priming dose consisted of 4.0 μmol/kg of L[1−¹³C] leucine and 0.11 mg/kg of NaH¹³CO₃ and a sustaining infusion rate of L[1−¹³C] leucine of 4.0 μmol/kg/h for 4 hours. The isotopes were purchased directly from Cambridge Isotope Laboratories.

A 1 mL sample of venous blood was taken before administration of the dose of isotope and at 180, 195, 210, 225 and 240 minutes after the start of infusion.

The blood samples was divided into equal parts and stored in plastic cryogenic tubes with a screw top. One sample was sent for leucine analysis; the other was retained in case of loss or breakage. All retained samples were destroyed once the database was locked.

Blood samples were stable for 6 months when stored at −20° C. Samples were packed in dry ice and shipped by overnight freight service to Metabolic Solutions Inc (460 Amherst Street, Nashua, USA,) for analysis.

Breath samples were provided by blowing through a straw into Exetainer® tubes (evacuated 139 B blue tops). Breath samples were collected before the dose of isotope and at 180, 195, 210, 225 and 240 minutes after the start of infusion. Duplicate samples were collected. One sample was sent for analysis; the other was retained in case of loss or breakage. All retained samples were destroyed once the database was locked.

Breath samples were stable for 3 months when stored at room temperature. Samples were packed at room temperature and shipped to Metabolic Solutions Inc for analysis.

### Subjective Global Assessment (SGA)

An SGA was an overall evaluation of a subject by an experienced clinician that correlated with the subjective and objective aspects of a medical history and physical examination. An SGA was completed at both the screening and baseline visits, on Day 28 and at the follow-up visit. Review of the medical history included an assessment of weight and weight change, dietary intake, gastrointestinal symptoms, disease state, and the subject's functional status. The SGA also included a physical examination for negative changes in body composition such as loss of subcutaneous fat or muscle wasting, and signs of oedema or ascites (nutrition-related). After evaluation, the subject was classified as well nourished (A), mild to moderately malnourished (B), or severely malnourished (C).

### Anthrompometry and Bioimpedance

Height (cm) was measured only at the screening visit. Anthrompometry was measured at every visit from baseline to follow-up. Anthropometric measurements included weight, skinfold thicknesses and arm circumference. Weight (kg) was measured using a high performance scale. Subjects were asked to stand on the scale with minimal clothing, no shoes, and with even weight on both feet. Skinfold thickness (mm) was measured by using Harpenden Skinfold Callipers at 4 sites: biceps, triceps, suprailiacal and subscapular. Arm circumference (cm) was measured using a tape measure that is flexible, inelastic and metric. The site measured was marked, and the tape was not to be too tight or too loose, and to lie flat on the skin, and be horizontal. Bioimpedance measurements were made to measure body composition at every visit from baseline to Day 28. The bioimpedance scale (RJL Systems) was used. Resistance, reactance and the phase angle were recorded.

### Dual X-Ray Absorptiometry (DEXA) Scan

Subjects had a whole body DEXA scan (%, kg) at baseline and Day 28. The DEXA scan was performed by an appropriately qualified person and in accordance with the agreed guidelines. Mean body composition values (fat free mass, fat mass and bone mineral content) were recorded for the total body and for defined regional areas (arms, legs, trunk), if possible.

### Blood Tests

The total volume of blood collected throughout the study was approximately 400 mL. Plasma proteins

Plasma proteins were measured as markers for renal disease and malnourishment. Blood samples were analysed locally. Albumin (g/L) were measured at screening, baseline and on Day 28. Blood samples for measurement of pre-albumin (g/L) were collected at baseline, Day 14 and Day 28. Transferrin (g/L) was measured at baseline, Day 7, Day 14 and Day 28.

### Glucose Control

HbA_{1c} (%) was measured at screening and Day 28 as a marker for long-term glucose control. Fasting insulin (μunits/mL) and fasting glucose (mmol/L) were measured at baseline, Day 7, Day 14 and Day 28 as markers for short-term glucose control.

### Lipids

Fasting triglyceride (mmol/L) and fasting cholesterol (mmol/L) were measured at baseline, Day 7, Day 14 and Day 28 as markers for lipid metabolism.

### Endocrine Testing

On Day 0 (baseline) and Day 28, subjects commenced 24-hour sampling at 10 and 20-minute intervals to construct a GH profile. Subjects had a plastic cannula inserted into a vein in the back of the hand or in the forearm to facilitate the collection of blood samples. The cannula was flushed with heparin-saline solution after each blood withdrawal to prevent clotting. Immediately before each blood collection, the residual heparin-saline volume instilled in the cannula was withdrawn with 0.5 mL blood in a separate syringe. This volume was reinjected after collection of the sample used for GH analysis.

At the baseline visit, blood sampling for the 24-hour GH profile commenced on Day 0 by taking samples at 20-minute intervals for 20 hours. Immediately at the end of this period, on the morning of Day 1, samples were taken at 10-minute intervals for 4 hours. The first dose of investigational product was not given before the end of the 24-hour GH profile period. On Day 28, samples were taken pre-dose, at 10-minute intervals for the first 4 hours (at the same time of day as the 4-hour period on Day 1) and at 20-minute intervals thereafter for a further 20 hours.

Samples were analysed for GH at the Department of Nephrology, Endocrinology and Metabolic Diseases, Silesian University School of Medicine, Katowice, Poland using a commercial kit.

### Insulin-Like Growth Factors (IGF-1)

Total IGF-1 (ng/mL), insulin-like growth factor binding protein (IGFBP-1 and IGFBP-3, ng/mL) were measured at every visit from baseline to Day 28 as markers for tissue and cell growth and proliferation. Samples were analysed at the Department of Nephrology, Endocrinology and Metabolic Diseases, Silesian University School of Medicine, Katowice using a Medgenix Diagnostics kit.

### Fat Regulation

Leptin (ng/mL) and adiponectin (μg/mL) were measured at every visit from baseline to Day 28 as markers for regulating metabolism. Samples were analysed at the Department of Nephrology, Endocrinology and Metabolic Diseases, Silesian University School of Medicine, Katowice.

### Hormones

A blood sample was taken at screening for analysis of TSH and free thyroxin. Women of childbearing potential had a urine pregnancy test at screening. The hormones testosterone (nmol/L) and oestradiol (nmol/L) were measured at baseline and on Day 28 because those could be affected by GH and could lead to secondary changes in body composition. Samples were analysed locally.

### 24-Hour Urine Collection

Subjects collected 24-hour urine samples at screening, baseline (Days 0 to 1) and Days 28 to 29. All of the samples were analysed for creatinine and urea clearance (mL/min). The samples collected at baseline and on Day 28 were also analysed for proteinuria (Coumassie method), PNA, sodium, potassium, chloride, calcium and phosphate. GFR was calculated using the Cockcroft-Gault formula:

$$Cockcroft \text{-} Gault GFR = \frac{[ 140 - age ( yrs ) ] \times [ weight ( kg ) ]}{\begin{matrix}72 \times [ Plasma creatinine ( mg \text{/} dl ) ] \times \\ 0.85 if female\end{matrix}}$$

Electrolyte excretion rates were calculated as follows:

[symbol]

$$Electrolyte excretion rate = \frac{\frac{Urinary concentration of electrolyte * urine volume}{Body weight}}{Collection time}$$

Electrolyte fractional excretion rates were calculated as follows:

$$Electrolyte fractional excretion rate = \frac{( \begin{matrix}Urinary concentration of electrolyte / \\ serum concentration of electrolyte\end{matrix} )}{( Serum creatinine / urinary creatinine )}$$

Aliquots of urine (at least 10 mL) were saved in case repeat measurements were necessary. Samples were analysed locally.

### Spontaneous Nutrient Intake

Dietary assessments were performed at the baseline visit and were repeated on Day 28. The subjects were asked to write down everything they ate and drank throughout the 3 days preceding the visit. The local dietician then calculated the mean amount of carbohydrates, fat and protein consumed by the subjects per day and calculated energy intake.

### Appropriateness of Measurements

Although leucine isotope was first described by R. Schoenheimer already in 1939, the use of leucine kinetics for studying the metabolic effects of a therapeutic intervention is relatively new and sensitive technique. The technique applied in this study measured total body protein turnover from circulating blood using branched amino acid leucine. Although other methods can also determine protein metabolism (nitrogen balance and dual tracer (combination of arteriovenous balance methods and tracer technique), this technique has been selected because it offers a more efficient safer method of measuring whole body protein turnover, provides a more detailed analysis and have been intensively used and reviewed in many human studies. Nitrogen balance is conceptually simple and is measured by the difference between total nitrogen intake and output, adjusting for changes in body urea nitrogen and unmeasured nitrogen losses. These estimates at steady state provide a net measurement of the protein accretion or loss, but it is very laborious and time consuming and no insight into the processes of protein synthesis and degradation at either the whole body or tissue level, while leucine kinetic tracer technique provides the most accurate method currently available to simultaneously estimate whole body protein synthesis and degradation. With this method it is also possible to estimate the dynamic changes in response to specific interventions. While the dual tracer technique allows measurement of protein turnover in specific tissues or organs, these methods require arterial sampling together with sampling of venous blood draining the tissues being studied, thus invasive to subjects. Although isotopic tracer method presents a better tool for measuring protein metabolism, there is a discrepancy in the outcome from different amino acid used. L[1−¹³C] leucine has been considered the method of reference, and many studies have been using it for chronic kidney disease studies. Thus one of the many reason leucine has been chosen for this study. Studies have shown that, when using leucine tracer to compare protein flux in predialysis CRF patients and normal subjects, no differences were found in protein flux. Many kinetic studies have shown no increase in protein breakdown or reduction in net protein balance in predialysis patients.

The 24-hour GH profile testing in this study is a direct measure of the efficacy of the GHRH(1-29)NH₂ synthetic analog in stimulating endogenous GH secretion. It has not been documented to date that GHRH receptors are not down regulated in patients with chronic kidney disease and that GH response to exogenously administered GHRH is not impaired in this patient population due to the disease. However, it has been found that GHRH-R is downregulated in a high-glucose environment in diabetic rats.

The more traditional methods of assessing metabolism and nutritional status (anthropometry, bioimpedance, DEXA scan, IGF-11, and plasma protein levels) were included in the study measures but were not expected to show a clear response to the relatively short exposure to treatment used in this study.

### Efficacy Variables

The primary measure of efficacy was the effect of treatment on protein turnover as assessed by ¹³C-leucine kinetics. The secondary efficacy variables were:
Changes in GH secretion, circulating free and total IGF-1, IGF-binding proteins -1 and -3.
Fat-free mass and fat mass (absolute and as % of body weight) as assessed by DEXA scan, anthropometry and bioimpedance.
Biochemical markers of nutritional and metabolic state as measured by serum proteins (albumin, pre-albumin, transferrin), fat regulation hormones (leptin, adiponectin), glucose control (HbA_{1c}, insulin, glucose), lipids (triglycerides, total cholesterol).
Nutrient intake as measured by 3-day dietary protocols.
Subjective global assessment (SGA).

### Statistical Methods Planned in the Protocol and Determination of Sample Size

### General Considerations

All hypothesis tests were performed with a two-sided significance level of α=0.05. All confidence intervals (CIs) were 95%. The study was powered to show the beneficial effect of the GHRH(1-29)NH₂ synthetic analog on protein turnover as measured by leucine oxidation. Because of the explorative nature of this study, there was no adjustment for testing multiple variables.

### Statistical Analysis Methods

Numerical data with one data point after baseline was planned to be analysed using analysis of covariance (ANCOVA) with baseline value as covariate and treatment and site as factors. If the assumption of (log) normality was questionable, non-parametric ANCOVA with baseline value as covariate was planned to be used instead.

If there were several data points after baseline, analysis of variance (ANOVA) for repeated measures was the method of choice. The ANOVA model included baseline as covariate, site, treatment and visit and treatment by visit interaction term as factors. If the assumption of (log) normality was questionable, non-parametric ANCOVA with baseline value as covariate per visit separately was used instead.

The final analysis method was selected as part of blind data review, and defined in the Statistical Analysis Plan, which was approved before opening the blind.

### Interim Analysis

One unplanned interim analysis was performed. An interim analysis plan was written to describe the reason for the unplanned interim analysis and the procedures to protect data integrity.

The interim analysis was conducted to speed up the development process of the investigational product. The clinical study conduct was completed at the time of the interim analysis, but the data cleaning process was ongoing. Therefore, the analysis was conducted confidentially, i.e. the treatment code was unblinded only for the independent statistician who performed the analysis. The data included in the interim analysis were the primary analysis results and, in particular, for the primary endpoint, as final results. For the selected variables p-value for treatment difference and point-estimate and 95% CI for the mean difference were reported to the study team. If a non-parametric analysis method was considered as primary, the point-estimate and 95% non-parametric CI for the median per treatment group and the p-value for the comparison between groups were reported. The results were supported with the mean by treatment group and visit and mean change from baseline to better understand the results.

The following data were reported in the interim analysis:
Leucine kinetics data normalised with body weight.
Leucine kinetics data normalised with fat free mass.
Total IGF-1.
GH concentration summarised as area under the curve (AUC), mean and standard deviation (SD) for 4 hours and 24 hours.
DEXA fat mass and fat free mass for total body.
Anthropometry: fat mass and fat free mass.

### Analysis Populations

### Intent-to-Treat (ITT)

The intent-to-treat (ITT) population was applied to demography and efficacy data. This analysis set included all subjects randomised.

### Safety

The safety analysis set was applied to safety data. This analysis set included all subjects randomised, to whom study drug was administrated at least once, and who provided at least some data after first treatment administration.

### Per Protocol (PP)

The per protocol (PP) population was applied to leucine kinetic data and to GH-secretion data. The decision to exclude any subject from the PP analysis was made by the sponsor, based on the blinded list of all protocol deviations. As a result of this review, the decision to have two PP datasets was made, i.e. one for leucine kinetics and one for GH-secretion data.

### Determination of Sample Size

This study was designed to show superiority of the GHRH(1-29)NH₂ synthetic analog over placebo on protein turnover as assessed by leucine oxidation rate of ¹³C-leucine kinetics. In another study the SD of the leucine oxidation rate varied between 9 to 17 μmol/kg in GH-deficient adults treated with GH replacement. Seventeen subjects in each group has 80% power to detect a difference in means of 15 μmol/kg (1 SD) assuming that the common SD is 15 when using a 2-group t-test with a 0.05 2-sided significance level. However, using covariate in the analysis, more power is gained. If the correlation coefficient of the covariate and the target variable is 0.5, the residual variance in the analysis is 25% smaller than if the covariate is not included. With that assumption for detecting the difference of 15 μmol/kg in leucine oxidation rate would require 13 subjects per group to maintain the power of 80% and 2-sided significance level of 0.05. As the correlation coefficient is unknown, by targeting to 13 subjects per group, a fairly conservative approach is applied.

An overall drop-out rate of about 20% was anticipated. Therefore, 16 subjects per group i.e. a total of 32 subjects were to be randomized.

### Disposition of Subjects

Twenty-eight subjects were randomized: 13 (46.4%) to the GHRH(1-29)NH₂ synthetic analog group and 15 (53.6%) to the placebo group. Twenty-six (92.9%) subjects completed the study as planned (Table 15). One subject from each group discontinued from the study. The reason for discontinuation in both cases was withdrawal of consent. However, the subject who withdrew from the placebo group also had an SAE of cardiac failure on the same date that consent was withdrawn.

TABLE 15
End of Study Information: All Subjects Randomised

| | |
|---|---|
| GHRH | Placebo |
| N = 13 | N = 15 |

| | n | (%) | n | (%) |
|---|---|---|---|---|
| Study completed as planned | 12 | (92.3%) | 14 | (93.3%) |
| Reason for withdrawal | | | | |
| Withdrawal of consent | 1 | (7.7%) | 1 | (6.7%) |

### Data Sets Analyzed

Four analysis populations were defined. All 28 subjects were included in the ITT and Safety populations; 27 (96.4%) subjects were included in the PP (GH Secretion) population and 21 (75.0%) subjects were included in the PP (Leucine Kinetics) population.

### Demographic Data

Fifteen (53.6%) subjects overall were male, with slightly more males in the placebo group compared with the GHRH(1-29)NH₂ synthetic analog group (Table 16). Mean age was 61.8 years in the GHRH(1-29)NH₂ synthetic analog group and 63.4 years in the placebo group, and ranged between 44 and 85 years overall. All of the subjects were Caucasian.

TABLE 16
Demographic and Baseline Characteristics: ITT Population

| | | GHRH | | Placebo | |
|---|---|---|---|---|---|
| | | N = 13 | | N = 15 | |
| Sex | Male (n, %) | 6 | (46.2%) | 9 | (60.0%) |
| | Female (n, %) | 7 | (53.8%) | 6 | (40.0%) |

| Age (years) | mean (SD) | 61.8 | (9.5) | 63.4 | (10.5) |
|---|---|---|---|---|---|
| | (range) | (44-76) | | (51-85) | |
| Height (cm) | mean (SD) | 163.2 | (15.5) | 168.5 | (9.0) |

| | | |
|---|---|---|
| (range) | (130-180) | (152-183) |

| | | | | |
|---|---|---|---|---|
| Ethnic group, Caucasian (n, %) | 13 | (100%) | 15 | (100%) |

### Medical History

All of the subjects reported 1 or more items of medical history and all had documented chronic renal failure. The 2 groups were generally well matched for medical history.

The most common Medical Dictionary for Regulatory Activities (MedDRA) Preferred Terms for medical history were hypertension (12 [92.3%] subjects GHRH(1-29)NH₂ synthetic analog; 14 [93.3%] subjects placebo) followed by anaemia (4 [30.8%] subjects GHRH(1-29)NH₂ synthetic analog; 8 [53.3%] subjects placebo) and hypercholesterolaemia (5 [38.5%] subjects GHRH(1-29)NH₂ synthetic analog; 2 [13.3%] subjects placebo).

### Concomitant Medication

All of the subjects were taking at least 1 concomitant medication and the 2 groups were generally well matched for medications.

The most common World Health Organisation Drug Dictionary (WHO-DD) Anatomic Therapeutic Chemical (ATC) concomitant medications were furosemide (12 [92.3%] subjects GHRH(1-29)NH₂ synthetic analog; 14 [93.3%] subjects placebo), followed by allopurinol (8 [61.5%] subjects GHRH(1-29)NH₂ synthetic analog; 10 [66.7%] subjects placebo) and calcium supplements (7 [53.8%] subjects GHRH(1-29)NH₂ synthetic analog; 10 [66.7%] subjects placebo).

### Urine Pregnancy Test

Urine pregnancy tests were conducted on 3 occasions. The results were negative on each occasion of testing.

### Primary Efficacy Variable: Leucine Oxidation Assessed by ¹³C-Leucine Kinetics

Mean and median leucine oxidation was higher at Day 28 compared with baseline in the GHRH(1-29)NH₂ synthetic analog group but was lower or with very small change in the placebo group (Table 17 and Table 18).

There was no statistically significant difference in the ITT population between treatments in mean leucine oxidation at Day 28 (p=0.750). The adjusted mean difference (GHRH(1-29)NH₂ synthetic analog-placebo) between the groups was 1.1 μmol/kg.hr (95% CI-6.0; 8.2). In the PP (Leucine Kinetic) population, there was no statistically significant difference between the treatments on mean leucine oxidation at Day 28 (p=0.139). The adjusted mean difference (GHRH(1-29)NH₂ synthetic analog-placebo) between the groups was 3.3 μmol/kg.hr (95% CI-1.2; 7.8 μmol/kg.hr).

The assumption of normality was questionable. Therefore a non-parametric analysis method was applied to the data. In this secondary analysis, statistically significant differences were detected between the groups in the ITT population (p=0.009) and in PP (Leucine Kinetic) population (p=0.040).

TABLE 17
Summary of Leucine Oxidation (μmol/kg · hr): ITT Population

| | GHRH | Placebo | Adjusted mean | | |
|---|---|---|---|---|---|
| | N = 13 | N = 15 | difference | 95% CI | P-value |
| Baseline, n | 13 | 15 | 1.1 | (−6.0; 8.2) | 0.750 |
| Mean (SD) | 7.74 (4.51) | 9.83 (6.90) | | | |
| Median | 5.8 | 8.8 | | | |
| Change from baseline to | | | | | |
| Day 28, n | 12 | 14 | | | |
| Mean (SD) | 2.38 (5.66) | 0.84 (10.66) | | | |
| Median | 2.3 | −1.3 | | | |

Treatment comparison with ANCOVA assuming normal distribution

TABLE 18
Summary of Leucine Oxidation (μmol/kg · hr): PP (Leucine Kinetic) Population

| | GHRH | Placebo | Adjusted mean | | |
|---|---|---|---|---|---|
| | N = 13 | N = 15 | difference | 95% CI | P-value |
| Baseline, n | 9 | 12 | 3.3 | (−1.2; 7.8) | 0.0.139 |
| Mean (SD) | 7.81 (5.42) | 10.04 (7.61) | | | |
| Median | 5.6 | 7.7 | | | |
| Change from baseline to | | | | | |
| Day 28, n | 9 | 11 | | | |
| Mean (SD) | 1.90 (6.24) | −1.42 (3.74) | | | |
| Median | 2.3 | −1.1 | | | |

Treatment comparison with ANCOVA assuming normal distribution

### Secondary Efficacy Variables: Other ¹³C Leucine Kinetic Data

The results of other leucine kinetic data normalised for body weight are summarised for the ITT population in Table 19.

At Day 28, mean leucine flux was 1.15 μmol/kg.hr higher than the baseline value of 101.46 μmol/kg.hr in the GHRH(1-29)NH₂ synthetic analog group but was 3.19 μmol/kg.hr lower than the baseline value of 94.01 μmol/kg.hr in the placebo group. The adjusted mean difference (95% CI) was 7.5 μmol/kg.hr (−12.0; 27.0) and was not statistically significant (p=0.431).

At Day 28, mean protein synthesis was 1.22 μmol/kg.hr lower than the baseline value of 93.71 μmol/kg.hr in the GHRH(1-29)NH₂ synthetic analog group and was 4.03 μmol/kg.hr lower than the baseline value of 84.19 μmol/kg.hr in the placebo group. The adjusted mean difference (95% CI) was 6.9 μmol/kg.hr (−10.9; 24.7) and was not statistically significant (p=0.431).

At Day 28, mean protein breakdown was 1.16 μmol/kg.hr higher than the baseline value of 97.45 μmol/kg.hr in the GHRH(1-29)NH₂ synthetic analog group but was 3.19 μmol/kg.hr lower than the baseline value of 90.01 μmol/kg.hr in the placebo group. The adjusted mean difference (95% CI) was 7.5 μmol/kg.hr (−11.9; 27.0) and was not statistically significant (p=0.430).

Median oxidation of flux was 3.8% higher at Day 28 than the baseline value in the GHRH(1-29)NH₂ synthetic analog group but was 1.9% lower than the baseline value in the placebo group. The adjusted mean difference (95% CI) was 1.48% (0.95; 2.30) and was not statistically significant (p=0.077).

Median protein synthesis of flux was 3.8% lower than the baseline value in the GHRH(1-29)NH₂ synthetic analog group but was 1.9% higher than the baseline value in the placebo group. The difference for the dose group comparisons was not statistically significant (p=0.061).

The findings of the PP (Leucine Kinetic) population supported the findings of the ITT population. Similarly, leucine kinetic data normalised for fat free mass supported the findings of leucine kinetic data normalised for body weight in both the ITT and PP (Leucine Kinetic) populations.

TABLE 19
Other Leucine Kinetics Results - Normalised With Body Weight: ITT Population

| | | | | |
|---|---|---|---|---|
| GHRH | Placebo | Adjusted mean | | |
| N = 13 | N = 15 | difference | 95% CI | P-value |

Leucine flux (μmol/kg · hr)

| | | | | | |
|---|---|---|---|---|---|
| Baseline, mean (SD) | 101.46 (21.54) | 94.01 (24.66) | | | |
| Median | 98.3 | 84.5 | | | |
| Mean (SD) change from | 1.15 (30.09) | −3.19 (25.50) | 7.5 | (−12.0; 27.0) | 0.431¹ |
| baseline to Day 28 | | | | | |
| Median change from | −0.4 | 3.6 | | | |
| baseline to Day 28 | | | | | |

Protein synthesis (μmol/kg · hr)

| | | | | | |
|---|---|---|---|---|---|
| Baseline, mean (SD) | 93.71 (20.38) | 84.19 (26.24) | | | |
| Median | 88.9 | 78.8 | | | |
| Mean (SD) change from | −1.22 (30.75) | −4.03 (24.93) | 6.9 | (−10.9; 24.7) | 0.4.13¹ |
| baseline to Day 28 | | | | | |
| Median change from | −4.6 | −1.3 | | | |
| baseline to Day 28 | | | | | |

Protein breakdown (μmol/kg · hr)

| | | | | | |
|---|---|---|---|---|---|
| Baseline, mean (SD) | 97.45 (21.53) | 90.01 (24.67) | | | |
| Median | 94.2 | 80.4 | | | |
| Mean (SD) change from | 1.16 (30.11) | −3.19 (25.52) | 7.5 | (−11.9; 27.0) | 0.430¹ |
| baseline to Day 28 | | | | | |
| Median change from | −0.4 | 3.4 | | | |
| baseline to Day 28 | | | | | |

Oxidation of flux (%)

| | | | | | |
|---|---|---|---|---|---|
| Baseline, mean (SD) | 7.61 (4.19) | 11.15 (8.94) | | | |
| Median | 6.9 | 10.0 | | | |
| Mean (SD) change from | 2.64 (6.26) | 0.91 (10.56) | 1.48 | (0.95; 2.30) | 0.077² |
| baseline to Day 28 | | | | | |
| Median change from | 3.8 | −1.9 | | | |
| baseline to Day 28 | | | | | |

Protein synthesis of flux (%)

| | | | | | |
|---|---|---|---|---|---|
| Baseline, mean (SD) | 92.39 (4.19) | 88.85 (8.94) | | | |
| Median | 93.1 | 90.0 | | | |
| Mean (SD) change from | −2.64 (6.26) | −0.91 (10.56) | Placebo | (89.3; 97.1) | 0.061³ |
| baseline to Day 28 | | | GHRH | (89.0; 94.3) | |
| Median change from | −3.8 | 1.9 | | | |
| baseline to Day 28 | | | | | |

¹Treatment comparison with ANCOVA assuming normal distribution
²Treatment comparison with ANCOVA assuming log-normal distribution
³Treatment comparison with non-parametric ANCOVA

### Secondary Efficacy Variables: Subjective Global Assessment (SGA)

At baseline, most subjects in each group were mild to moderately malnourished (Table 20). There was a small imbalance between the groups at baseline in that 1 (7.7%) subject in the GHRH(1-29)NH₂ synthetic analog group was severely malnourished compared with 4 (26.7%) subjects in the placebo group.

One subject in each group who had been mild to moderately malnourished at baseline, withdrew from the study and did not provide Day 28 data.

At Day 28, subjects were better nourished in the GHRH(1-29)NH₂ synthetic analog group than in the Placebo group. The number of well nourished subjects in the GHRH(1-29)NH₂ synthetic analog group was 9 (75.0%) compared with no change in the placebo group (3 [21.4%] subjects). The difference between the groups was statistically significant (p=0.0083; non-parametric ANCOVA).

TABLE 20
Subjective Global Assessment at Baseline and Day 28: ITT Population

| | GHRH | | Placebo | |
|---|---|---|---|---|
| | N = 13 | | N = 15 | |
| | n | (%) | n | (%) |
| Baseline | | | | |
| Well nourished | 3 | (23.1%) | 3 | (20.0%) |
| Mild to moderately malnourished | 9 | (69.2%) | 8 | (53.3%) |
| Severely malnourished | 1 | (7.7%) | 4 | (26.7%) |
| Day 28 | | | | |
| Well nourished | 9 | (75.0%) | 3 | (21.4%) |
| Mild to moderately malnourished | 2 | (16.7%) | 8 | (57.1%) |
| Severely malnourished | 1 | (8.3%) | 3 | (21.4%) |

### Secondary Efficacy Variables: Anthropometry

There was a statistically significant difference between the groups in mean body weight overall (p=0.019) (Table 21). The subjects in the GHRH(1-29)NH₂ synthetic analog group were, on average, 0.96 kg (95% CI-0.17; 1.75) heavier that the subjects in placebo group.

There were no statistically significant differences between the groups at Day 28 in any of the anthropometry measures.

Median fat mass decreased between baseline (15.8 kg) and Day 28 (15.6 kg) in the GHRH(1-29)NH₂ synthetic analog group but increased in the placebo group (14.4 kg at baseline, 14.9 kg at Day 28). However, median percent fat m the GHRH(1-29)NH₂ synthetic analog group and from 23.7% at baseline to 24.6% at Day 28 in the placebo group).

Mean fat free mass decreased from 46.64 kg at baseline to 46.20 kg at Day 28 in the GHRH(1-29)NH₂ synthetic analog group and increased from 49.32 kg at baseline to 49.46 kg at Day 28 in the placebo group.

Arm circumference and subscapular skinfold thickness increased between baseline and Day 28 in both groups. Biceps skinfold thickness decreased between baseline and Day 28 in the GHRH(1-29)NH₂ synthetic analog group and increased in the placebo group, whereas both triceps and suprailiacal skinfold thicknesses increased between baseline and Day 28 in the GHRH(1-29)NH₂ synthetic analog group and decreased in the placebo group.

TABLE 21
Anthropometry at Baseline and Day 28: ITT Population

| | | | | |
|---|---|---|---|---|
| GHRH | Placebo | Adjusted difference | | |
| N = 13 | N = 15 | or comparison | 95% CI | P-value |

Body weight (kg): mean (SD)

| | | | | | |
|---|---|---|---|---|---|
| Baseline | 62.84 (9.98) | 64.60 (8.06) | | | |
| Day 28 | 63.21 (10.45) | 64.97 (7.74) | 0.96 | (0.17; 1.75) | 0.019¹ |

Fat mass (kg): median

| | | | | | |
|---|---|---|---|---|---|
| Baseline | 15.8 | 14.4 | Placebo | (13.93; 20.27) | 0.662² |
| Day 28 | 15.6 | 14.9 | GHRH | (11.69; 22.77) | |

Fat mass (%): median

| | | | | | |
|---|---|---|---|---|---|
| Baseline | 25.4 | 23.7 | Placebo | (19.05; 30.05) | 0.227² |
| Day 28 | 25.9 | 24.6 | GHRH | (20.06; 37.93) | |

Fat free mass (kg): mean (SD)

| | | | | | |
|---|---|---|---|---|---|
| Baseline | 46.64 (9.13) | 49.32 (7.84) | | | |
| Day 28 | 46.20 (9.16) | 49.46 (8.60) | 0.41 | (−0.83; 1.66) | 0.501¹ |

Arm circumference (cm): mean (SD)

| | | | | | |
|---|---|---|---|---|---|
| Baseline | 26.35 (5.42) | 26.50 (4.52) | | | |
| Day 28 | 27.33 (4.81) | 27.11 (4.02) | 0.33 | (−0.18; 0.83) | 0.197¹ |

Skinfold thickness biceps (mm): median

| | | | | | |
|---|---|---|---|---|---|
| Baseline | 5.1 | 8.0 | Placebo | (6.00; 14.00) | 0.090² |
| Day 28 | 4.1 | 8.6 | GHRH | (3.50; 17.00) | |

Skinfold thickness triceps (mm): median

| | | | | | |
|---|---|---|---|---|---|
| Baseline | 12.0 | 11.0 | ratio | | 0.460³ |
| Day 28 | 13.3 | 10.7 | 1.066 | (0.894; 1.271) | |

Skinfold thickness suprailiacal (mm): median

| | | | | | |
|---|---|---|---|---|---|
| Baseline | 7.0 | 5.0 | Placebo | (3.20; 8.95) | 0.064² |
| Day 28 | 7.3 | 4.7 | GHRH | (4.80; 16.00) | |

Skinfold thickness subscapular (mm): median

| | | | | | |
|---|---|---|---|---|---|
| Baseline | 8.0 | 8.4 | Placebo | (7.50; 11.10) | 0.280² |
| Day 28 | 8.6 | 8.7 | GHRH | (6.20; 14.00) | |

¹ANOVA for repeated measures, assuming normal distribution
²Treatment comparison with non-parametric ANCOVA
³Treatment comparison with ANCOVA, assuming log-normal distribution

### Secondary Efficacy Variables: Bioimpedance

There was a statistically significant difference between groups in median fat mass (kg) on Day 7 (p=0.037), Day 21 (p=0.001) and Day 28 (p=0.008) and but not on Day 14 (p=0.105). Median fat mass was lower in the GHRH(1-29)NH₂ synthetic analog group at all visits (Table 22). The results obtained from estimation of fat mass (%) and fat free mass (kg) were similar to fat mass (kg), i.e. a statistically significant difference was detected on all study days except Day 14, and the median fat mass (%) and median fat free mass (kg) was lower in the GHRH(1-29)NH₂ synthetic analog group at all visits.

There were also statistically significant differences between the groups in extracelluar cell mass (p=0.076), extracellular water (p=0.000), intracellular water (p=0.227) and total body water (p=0.017) at Day 28.

TABLE 22
Bioimpedance at Baseline and Day 28: ITT Population

| | | | | |
|---|---|---|---|---|
| | | Comparison | | |
| GHRH | Placebo | between | | P- |
| N = 13 | N = 15 | groups | 95% CI | value¹ |

Fat mass (kg): median

| | | | | | |
|---|---|---|---|---|---|
| Baseline | 16.0 | 14.1 | Placebo | (14.43; 20.32) | 0.008 |
| Day 28 | 12.2 | 16.5 | GHRH | (10.61; 20.40) | |

Fat mass (%): median

| | | | | | |
|---|---|---|---|---|---|
| Baseline | 24.7 | 22.0 | Placebo | (21.70; 31.17) | 0.004 |
| Day 28 | 21.4 | 26.5 | GHRH | (16.38; 33.38) | |

Fat free mass (kg): median

| | | | | | |
|---|---|---|---|---|---|
| Baseline | 48.8 | 51.7 | Placebo | (40.80; 56.00) | 0.015 |
| Day 28 | 51.1 | 52.0 | GHRH | (41.60; 54.35) | |

Fat free mass (%): median

| | | | | | |
|---|---|---|---|---|---|
| Baseline | 75.3 | 78.0 | — | — | — |
| Day 28 | 78.6 | 73.6 | — | — | |

Resistance (ohm): median

| | | | | | |
|---|---|---|---|---|---|
| Baseline | 533.0 | 502.0 | — | — | — |
| Day 28 | 497.0 | 556.0 | — | — | |

Reactance (ohm): median

| | | | | | |
|---|---|---|---|---|---|
| Baseline | 58.2 | 48.9 | — | — | — |
| Day 28 | 48.6 | 61.9 | — | — | |

Phase angle (degrees): median

| | | | | | |
|---|---|---|---|---|---|
| Baseline | 6.0 | 5.6 | Placebo | (5.20; 7.10) | 0.042 |
| Day 28 | 5.6 | 6.3 | GHRH | (4.71; 6.41) | |

Body cell mass (kg): median

| | | | | | |
|---|---|---|---|---|---|
| Baseline | 23.0 | 25.0 | Placebo | (18.40; 29.34) | 0.076 |
| Day 28 | 22.6 | 24.6 | GHRH | (21.40; 28.69) | |

Extracellular cell mass (kg): median

| | | | | | |
|---|---|---|---|---|---|
| Baseline | 23.5 | 25.7 | Placebo | (20.40; 28.80) | 0.004 |
| Day 28 | 24.7 | 23.6 | GHRH | (19.73; 29.22) | |

Extracellular water (L): median

| | | | | | |
|---|---|---|---|---|---|
| Baseline | 17.7 | 18.1 | Placebo | (16.40; 19.30) | 0.000¹ |
| Day 28 | 19.1 | 17.5 | GHRH | (15.80; 23.30) | |

Intracellular water (L): median

| | | | | | |
|---|---|---|---|---|---|
| Baseline | 17.0 | 18.2 | Placebo | (13.90; 22.00) | 0.230 |
| Day 28 | 15.8 | 18.0 | GHRH | (14.59; 20.68) | |

Total body water (L): median

| | | | | | |
|---|---|---|---|---|---|
| Baseline | 35.8 | 38.0 | Placebo | (28.90; 40.40) | 0.017 |
| Day 28 | 38.1 | 37.4 | GHRH | (30.40; 39.95) | |

Basal metabolic rate (kcal): median

| | | | | | |
|---|---|---|---|---|---|
| Baseline | 1441 | 1479 | Placebo | — | — |
| Day 28 | 1473 | 1445 | GHRH | — | |

¹Non-parametric ANCOVA, baseline as covariate

### Secondary Efficacy Variables: DEXA Scan

There was a statistically significant difference between the 2 groups in mean fat mass (kg) on Day 28 (p=0.029) (Table 23). The mean fat mass in the GHRH(1-29)NH₂ synthetic analog group was 84% of the mean fat mass in the placebo group (95% CI; 0.72-0.98). Similar results were obtained for fat mass (%) at Day 28 (p=0.014). The mean fat mass (%) for the GHRH(1-29)NH₂ synthetic analog group was 83% of the mean in the placebo group (95% CI; 71%-96%).

Statistically significant differences were also observed in fat free mass (kg) (p=0.022). The mean fat free mass (kg) was 2.9 kg higher in the GHRH(1-29)NH₂ synthetic analog group compared with the Placebo group on Day 28 (95% CT; 0.5-5.4 kg).

Total body bone mineral content increased between baseline and Day 28 in the GHRH(1-29)NH₂ synthetic analog group and decreased in the placebo group. The increase in bone mineral content occurred only in the trunk: mean bone mineral content in the trunk was 0.0699 kg at baseline and 0.718 kg at Day 28 in the GHRH(1-29)NH₂ synthetic analog group compared with 0.700 kg both at baseline and at Day 28 in the placebo group.

TABLE 23
DEXA Scan Results at Baseline and Day 28: ITT Population

| | | | | |
|---|---|---|---|---|
| GHRH | Placebo | | | |
| N = 13 | N = 15 | Ratio of means | 95% CI | P-value¹ |

Total body - fat mass (kg): mean (SD)

| | | | | | |
|---|---|---|---|---|---|
| Baseline | 17.10 (9.48) | 16.26 (6.13) | | | |
| Day 28 | 16.51 (9.51) | 17.84 (7.52) | 0.84 | (0.72; 0.98) | 0.029 |

Arms - fat mass (kg): mean (SD)

| | | |
|---|---|---|
| Baseline | 2.22 (1.65) | 1.87 (1.10) |
| Day 28 | 2.01 (1.25) | 2.28 (1.05) |

Legs - fat mass (kg): mean (SD)

| | | |
|---|---|---|
| Baseline | 5.26 (2.86) | 5.16 (1.97) |
| Day 28 | 5.31 (3.05) | 5.68 (2.61) |

Trunk - fat mass (kg): mean (SD)

| | | |
|---|---|---|
| Baseline | 8.52 (4.73) | 8.15 (3.16) |
| Day 28 | 8.17 (4.91) | 8.70 (3.79) |

Total body - fat mass (%): mean (SD)

| | | | | | |
|---|---|---|---|---|---|
| Baseline | 27.89 (14.25) | 26.01 (8.99) | | | |
| Day 28 | 26.84 (14.31) | 28.50 (10.74) | 0.83 | (0.71; 0.96) | 0.014 |

Total body - fat free mass (kg): mean (SD)

| | | | | | |
|---|---|---|---|---|---|
| Baseline | 43.83 (10.90) | 46.36 (8.33) | | | |
| Day 28 | 44.85 (11.61) | 44.66 (9.04) | 2.94 | (0.48; 5.40) | 0.022 |

Total body - fat free mass (%): mean (SD)

| | | |
|---|---|---|
| Baseline | 72.11 (14.25) | 73.99 (8.99) |
| Day 28 | 73.16 (14.31) | 71.50 (10.74) |

Total body - bone mineral content (kg): mean (SD)

| | | |
|---|---|---|
| Baseline | 2.236 (0.656) | 2.362 (0.463) |
| Day 28 | 2.225 (0.674) | 2.353 (0.483) |

Arms - bone mineral content (kg): mean (SD)

| | | |
|---|---|---|
| Baseline | 0.317 (0.109) | 0.339 (0.100) |
| Day 28 | 0.300 (0.106) | 0.332 (0.097) |

Legs - bone mineral content (kg): mean (SD)

| | | |
|---|---|---|
| Baseline | 0.791 (0.273) | 0.870 (0.200) |
| Day 28 | 0.784 (0.281) | 0.869 (0.207) |

Trunk - bone mineral content (kg): mean (SD)

| | | |
|---|---|---|
| Baseline | 0.699 (0.239) | 0.700 (0.164) |
| Day 28 | 0.718 (0.253) | 0.700 (0.178) |

Total body - bone mineral content (%): mean (SD)

| | | | |
|---|---|---|---|
| Baseline | 3.63 (0.68) | 3.78 (0.63) | |
| Day 28 | 3.58 (0.69) | 3.77 (0.61) | (;) |

¹ANOVA for repeated measures, assuming normal distribution

### Secondary Efficacy Variables: Biochemical Markers of Nutritional and Metabolic State

Statistically significant differences in fasting insulin and fasting cholesterol between 2 groups were observed (Table 24). Median fasting insulin concentrations were consistently higher for the GHRH(1-29)NH₂ synthetic analog group compared with the placebo group (95% CI; CI: 1.131-2.829, p=0.015). Similarly, fasting cholesterol concentrations were higher for the GHRH(1-29)NH₂ synthetic analog group compared with the placebo group (95% CI; −1.02-−0.13, p=0.014).

There were no statistically significant differences between the 2 groups in albumin, pre-albumin, transferrin, HbA_{1c}, fasting glucose, or fasting triglycerides.

TABLE 24
Biochemical Markers of Nutritional and Metabolic State: ITT Population

| | | | | |
|---|---|---|---|---|
| GHRH | Placebo | Comparison or | | |
| N = 13 | N = 15 | ratio of means | 95% CI | P-value |

Albumin (g/L): median

| | | | | | |
|---|---|---|---|---|---|
| Baseline | 41.0 | 41.5 | Placebo | (33.30; 47.57) | 0.425¹ |
| Day 28 | 40.4 | 40.3 | GHRH | (35.20; 41.61) | |

Pre-albumin (g/L): mean (SD)

| | | | | | |
|---|---|---|---|---|---|
| Baseline | 0.216 (0.088) | 0.239 (0.148) | −0.093 | (−0.196; 0.011) | 0.076² |
| Day 28 | 0.201 (0.103) | 0.259 (0.112) | | | |

Transferrin (g/L): mean (SD)

| | | | | | |
|---|---|---|---|---|---|
| Baseline | 2.202 (0.315) | 2.228 (0.567) | 0.06 | (−0.06; 0.18) | 0.325² |
| Day 28 | 2.383 (0.370) | 2.267 (0.472) | | | |

HbA_{1c} (%): median

| | | | | | |
|---|---|---|---|---|---|
| Screening | 5.516 | 5.538 | Placebo | (4.890; 5.970) | 0.157¹ |
| Day 28 | 5.606 | 5.352 | GHRH | (4.927; 6.500) | |

Fasting insulin (μ units/mL): median

| | | | | | |
|---|---|---|---|---|---|
| Baseline | 8.4 | 5.8 | 1.788 | (1.131; 2.829) | 0.015³ |
| Day 28 | 12.1 | 7.0 | | | |

Fasting glucose (mmol/L): median

| | | | | | |
|---|---|---|---|---|---|
| Baseline | 5.1 | 5.1 | 1.030 | (0.934; 1.135) | 0.537³ |
| Day 28 | 5.4 | 5.0 | | | |

Fasting triglycerides (mmol/L): median

| | | | | | |
|---|---|---|---|---|---|
| Baseline | 1.40 | 1.62 | 1.199 | (0.957; 1.502) | 0.110³ |
| Day 28 | 1.98 | 1.43 | | | |

Fasting cholesterol (mmol/L): median

| | | | | | |
|---|---|---|---|---|---|
| Baseline | 1.40 | 1.62 | −0.58 | (−1.02; −0.13) | 0.014³ |
| Day 28 | 1.98 | 1.43 | | | |

¹Treatment comparison with non-parametric ANCOVA
²ANOVA for repeated measures, assuming normal distribution
³Treatment comparison with ANCOVA, assuming log-normal distribution

### Secondary Efficacy Variables: GH Profile

GH secretion

AUC was significantly greater for the GHRH(1-29)NH₂ synthetic analog group at 4 h (95% CI; 4.4-20.3, p=0.000) and 24 h (95% CI; 4571-18598, p=0.003) compared with the placebo group (Table 25). Median GH concentrations were significantly higher for the GHRH(1-29)NH₂ synthetic analog group at 4 h (95% CI; 4.4-20.3, p=0.000) and 24 h (95% CI; 3.8-15.3, p=0.002) compared with the placebo group.

The findings of the PP population supported those of the ITT population.

TABLE 25
GH Secretion Parameters: ITT Population

| | | | | |
|---|---|---|---|---|
| GHRH | Placebo | Comparison or | | |
| N = 13 | N = 15 | ratio of means | 95% CI | P-value |

AUC (μIU/mL × min) at 4 h: median

| | | | | | |
|---|---|---|---|---|---|
| Baseline | 315 | 278 | Ratio | | |
| Day 28 | 3891 | 299 | 9.49 | (4.4; 20.3) | 0.000¹ |

AUC (μIU/mL × min) at 24 h: median

| | | | | | |
|---|---|---|---|---|---|
| Baseline | 3473 | 3146 | | | |
| Day 28 | 16753 | 3070 | 11585 | (4571; 18598) | 0.003² |

concentration (μIU/mL) at 4 h: median

| | | | | | |
|---|---|---|---|---|---|
| Baseline | 1.3 | 1.2 | ratio | | |
| Day 28 | 16.2 | 1.2 | 9.50 | (4.4; 20.3) | 0.000¹ |

concentration (μIU/mL) at 24 h: median

| | | | | | |
|---|---|---|---|---|---|
| Baseline | 2.1 | 2.1 | | | |
| Day 28 | 12.5 | 2.1 | 9.5 | (3.8; 15.3) | 0.002² |

SD (μIU/mL) at 4 h: median

| | | | | | |
|---|---|---|---|---|---|
| Baseline | 1.0 | 0.9 | | | |
| Day 28 | 11.6 | 1.2 | 6.97 | (3.2; 15.2) | 0.000¹ |

SD (μIU/mL) at 24 h: median

| | | | | | |
|---|---|---|---|---|---|
| Baseline | 2.9 | 2.0 | | | |
| Day 28 | 13.3 | 2.4 | 3.92 | (2.1; 7.4) | 0.000² |

¹Treatment comparison with non-parametric ANCOVA
²ANOVA for repeated measures, assuming normal distribution

### GH Deconvolution

On Day 28, the second component GH half-life was longer for the GHRH(1-29)NH₂ synthetic analog group (95% CI; 0.6-9.0, p=0.029) compared with the placebo group (Table 26). The time from onset to peak of GH burst was shorter for the GHRH(1-29)NH₂ synthetic analog group (95% CI; −9.0-−0.1, p=0.048); basal GH secretion rate was greater for the GHRH(1-29)NH₂ synthetic analog group (95% CI; 1.34-4.79, p=0.007); pulsatile GH secretion rate was greater for the GHRH(1-29)NH₂ synthetic analog group (95% CI; 1.15-6.89, p=0.026); total GH secretion rate was greater for the GHRH(1-29)NH₂ synthetic analog group (95% CI; 1.23-5.99, p=0.016); and mass per pulse was greater for the GHRH(1-29)NH₂ synthetic analog group (95% CI; 7.2-45.8, p=0.009).

TABLE 22
GH Deconvolution: ITT Population

| | | | | |
|---|---|---|---|---|
| GHRH | Placebo | Comparison or ratio | | |
| N = 13 | N = 15 | of means | 95% CI | P-value |

Number of pulses: mean (SD)

| | | | | | |
|---|---|---|---|---|---|
| Baseline | 8.8 (3.2) | 11.3 (4.3) | | | |
| Day 28 | 9.7 (3.7) | 10.0 (3.0) | 0.3 | (−2.6; 3.2) | 0.824¹ |

1st component half-life (min): median

| | | | | | |
|---|---|---|---|---|---|
| Baseline | 6 | 3 | Placebo | (2; 6) | 0.815³ |
| Day 28 | 2 | 3 | GHRH | (2; 6) | |

2nd component half-life (min): median)

| | | | | | |
|---|---|---|---|---|---|
| Baseline | 16 | 18 | | | |
| Day 28 | 26 | 16 | 4.8 | (0.6; 9.0) | 0.029¹ |

Time from onset to peak of burst (min): mean (SD)

| | | | | | |
|---|---|---|---|---|---|
| Baseline | 32.8 (3.9) | 32.7 (7.4) | | | |
| Day 28 | 27.3 (4.7) | 32.0 (5.4) | −4.5 | (−9.0; −0.1) | 0.048¹ |

Basal secretion rate (mU/L/24 h): median

| | | | | | |
|---|---|---|---|---|---|
| Baseline | 27 | 11 | | | |
| Day 28 | 98 | 24 | 2.53 | (1.34; 4.79) | 0.007² |

Pulsatile secretion rate (mU/L/24 h): median

| | | | | | |
|---|---|---|---|---|---|
| Baseline | 100 | 58 | | | |
| Day 28 | 378 | 84 | 2.81 | (1.15; 6.89) | 0.026² |

Total secretion rate (mU/L/24 h): median

| | | | | | |
|---|---|---|---|---|---|
| Baseline | 120 | 73 | | | |
| Day 28 | 495 | 109 | 2.72 | (1.23; 5.99) | 0.016² |

Mass per pulse (mU/L): mean (SD)

| | | | | | |
|---|---|---|---|---|---|
| Baseline | 11.30 (7.02) | 8.92 (9.35) | | | |
| Day 28 | 43.88 (36.16) | 10.83 (14.83) | 26.5 | (7.2; 45.8) | 0.009¹ |

Mean pulse frequency (per 24 h): mean (SD)

| | | | | | |
|---|---|---|---|---|---|
| Baseline | 8.09 (2.81) | 10.54 (3.72) | | | |
| Day 28 | 9.07 (3.49) | 8.76 (3.19) | 0.8 | (−2.2; 3.8) | 0.577¹ |

Inter-pulse regularity: mean (SD)

| | | | | | |
|---|---|---|---|---|---|
| Baseline | 2.35 (0.63) | 2.87 (2.29) | | | |
| Day 28 | 1.95 (0.30) | 2.01 (0.66) | −0.0 | (−0.5; 0.4) | 0.8770¹ |

% pulsatile secretion rate: mean (SD)

| | | | | | |
|---|---|---|---|---|---|
| Baseline | 79.02 (9.09) | 75.18 (22.11) | | | |
| Day 28 | 71.27 (16.24) | 70.15 (14.47) | −0.4 | (−11.6; 10.8) | 0.945¹ |

¹ANOVA for repeated measures, assuming normal distribution
²Treatment comparison with non-parametric ANCOVA
³Treatment comparison with ANCOVA, assuming log-normal distribution

### Total IGF-1 Secondary Efficacy Variables: Insulin-Like Growth Factors

Median IGF-1 concentrations in the GHRH(1-29)NH₂ synthetic analog group were statistically significantly higher compared with those in the placebo group on every occasion of measurement (Table 27).

TABLE 27
Insulin-Like Growth Factors (IGF-1): ITT Population

| | Total IGF-1 (ng/mL) | | |
|---|---|---|---|
| | Median (95% CI) | | |
| | GHRH | Placebo | |
| Timepoint | N = 13 | N = 15 | P-value¹ |
| Baseline | 335.8 (—) | 384.8 (—) | |
| Day 7 | 576.0 (494.10; 828.70) | 337.0 (320.10; 445.20) | 0.001 |
| Day 14 | 611.8 (490.40; 913.60) | 356.0 (261.70; 424.80) | 0.001 |
| Day 21 | 729.8 (497.10; 913.50) | 347.2 (297.00; 552.20) | <0.0001 |
| Day 28 | 684.2 (564.10; 881.80) | 398.3 (334.10; 501.00) | <0.0001 |

¹Treatment comparison with non-parametric ANCOVA

### IGFBP

Mean IGFBP-1 concentrations in the placebo group were statistically significantly greater on Day 28 compared with the GHRH(1-29)NH₂ synthetic analog group (mean difference 28.40 ng/mL; 95% CI; −50.52-6.29; p=0.014) (Table 28). Mean concentration of IGFBP-1 in the GHRH(1-29)NH₂ synthetic analog group decreased while in the placebo group it increased on Day 28 from baseline. Differences between the 2 groups on Day 28 in IGFBP-3 concentrations were not statistically significantly different (mean difference 580.85 ng/mL; 95% CI; −150.81-1055.71; p=0.134) but they increased in both groups from baseline on Day 28.

TABLE 28
Insulin-Like Growth Factors Binding Proteins (IGFBP-1 and IGFBP-3): ITT Population

| | | | | |
|---|---|---|---|---|
| | | Adjusted | | |
| GHRH | Placebo | difference or | | |
| N = 13 | N = 15 | comparison | 95% CI | P-value¹ |

IGFBP-1 (ng/mL): mean (SD)

| | | | | | |
|---|---|---|---|---|---|
| Baseline | 74.98 (39.13) | 75.81 (25.30) | | | |
| Day 28 | 54.94 (30.72) | 77.65 (36.40) | −23.89 | (−42.44; −5.33) | 0.014 |

IGFBP-3 (ng/mL): median

| | | | | | |
|---|---|---|---|---|---|
| Baseline | 4039.6 (813.7) | 3879.6 (1893.4) | | | |
| Day 28 | 5130.8 (961.0) | 4425.0 (1013.7) | 580.85 | (−150.81; 1055.71) | 0.134 |

¹ANOVA for repeated measures, assuming normal distribution

### Secondary Efficacy Variables: Fat Regulation

### Leptin

Differences between the 2 groups on Day 28 in leptin concentrations were not statistically significantly different (ratio of mean difference 0.866 ng/mL; 95% CI; 0.424-1.771; p=0.681) but concentration of leptin in the GHRH(1-29)NH₂ synthetic analog group decreased while in the placebo group it increased on Day 28 from baseline (Table 29).

TABLE 29
Leptin: ITT Population

| | | | | |
|---|---|---|---|---|
| GHRH | Placebo | | | |
| N = 13 | N = 15 | Ratio of means | 95% CI | P-value¹ |

Leptin (ng/mL): mean (SD)

| | | | | | |
|---|---|---|---|---|---|
| Baseline | 23.80 (34.29) | 12.82 (14.85) | | | |
| Day 28 | 16.81 (21.81) | 14.28 (19.80) | 0.866 | (0.424; 1.771) | 0.681 |

¹Treatment comparison with ANCOVA, assuming log-normal distribution

### Adiponectin

Differences between the 2 groups in median leptin concentrations were not statistically significantly different on any occasion of measurement (Table 30).

TABLE 30
Adiponectin: ITT Population

| | Adiponectin (ng/mL) | | |
|---|---|---|---|
| | Median (95% CI) | | |
| | GHRH | Placebo | |
| Timepoint | N = 13 | N = 15 | P-value¹ |
| Baseline | 20604 | 20400 | — |
| Day 7 | 21293 (14535; 27438) | 18360 (13668; 31875) | 0.083 |
| Day 14 | 21548 (15810; 27030) | 20094 (14892; 29580) | 0.341 |
| Day 21 | 21905 (15810; 31365) | 18309 (15504; 31365) | 0.061 |
| Day 28 | 20553 (16320; 27030) | 20910 (14586; 32130) | 0.777 |

¹Treatment comparison with non-parametric ANCOVA

### Secondary Efficacy Variables: Hormones

TSH and free thyroxin were measured only in samples collected on Day-7 (Table 31) (to fulfil exclusion criteria).

At baseline, mean testosterone was higher in placebo-treated subjects (3.134 ng/mL) compared with the GHRH(1-29)NH₂ synthetic analog-treated subjects (1.550 ng/mL). On Day 28, mean testosterone was higher in placebo-treated subjects (2.983 ng/mL) compared with the GHRH(1-29)NH₂ synthetic analog-treated subjects (1.032 ng/mL).

There was little difference between the groups in mean oestradiol at baseline (30.400 pg/mL in the GHRH(1-29)NH₂ synthetic analog group; 31.983 pg/mL in the placebo group). At Day 28 mean oestradiol had almost halved compared with baseline in the GHRH(1-29)NH₂ synthetic analog group (16.281 pg/mL) but there was only a small decrease in placebo-treated subjects (28.500 pg/mL).

TABLE 31
Thyroid and Sex Hormones: ITT Population

| | | GHRH | Placebo |
|---|---|---|---|
| Hormone: mean (SD) | | N = 13 | N = 15 |
| TSH (microIU/mL) | Day −7 | 3.501 (6.925) | 2.277 (1.024) |
| Free Thyroxin (ng/dL) | Day −7 | 1.092 (0.266) | 1.057 (0.204) |
| Testosterone (ng/mL) | Day 0 | 1.550 (1.974) | 3.134 (3.664) |
| | Day 28 | 1.032 (1.327) | 2.983 (3.406) |
| Oestradiol (pg/mL) | Day 0 | 30.400 (23.333) | 31.983 (13.329) |
| | Day 28 | 16.281 (10.896) | 28.500 (18.525) |

### Secondary Efficacy Variables: 24-Hour Urine Samples

### Creatinine Clearance, Urea Clearance, and GFR

Creatinine clearance at baseline was higher in the GHRH(1-29)NH₂ synthetic analog group compared with the placebo group (Table 32). Median creatinine clearance was 24.92 mL/min/1.73 m⁻² at baseline and 23.78 mL/min/1.73 m⁻² on Day 28 for the GHRH(1-29)NH₂ synthetic analog group, and 19.93 mL/min/1.73 m⁻² at baseline and 15.81 mL/min/1.73 m⁻² on Day 28 for the placebo group. However, the difference was not statistically significant (95% CI; 0.83-1.51, p=0.451).

Urea clearance rate was sustained between baseline and Day 28 in the GHRH(1-29)NH₂ synthetic analog group but fell in the placebo group. Mean urea clearance rate was 11.86 mL/min/1.73 m⁻² at baseline and 11.24 mL/min/1.73 m⁻² on Day 28 for the GHRH(1-29)NH₂ synthetic analog group and 11.77 mL/min/1.73 m⁻² at baseline and 8.960 mL/min/1.73 m⁻² on Day 28 for the placebo group. However, the difference was not statistically significant (95% CI; −0.57-5.13, p=0.111).

GFR was sustained between baseline and Day 28 in the GHRH(1-29)NH₂ synthetic analog group but fell in the placebo group. Median GFR was 19.37 mL/min/1.73 m⁻² at baseline and 18.23 mL/min/1.73 m⁻² on Day 28 for the GHRH(1-29)NH₂ synthetic analog group and 18.10 mL/min/1.73 m⁻² at baseline and 13.54 mL/min/1.73 m⁻² on Day 28 for the placebo group. However, the difference was not statistically significant (p=0.777).

TABLE 32
Creatinine and Urea Clearance and GFR: ITT Population

| | | | | |
|---|---|---|---|---|
| GHRH | Placebo | Adjusted difference | | |
| N = 13 | N = 15 | or comparison | 95% CI | P-value |

nPNA (g/kg body weight/24 h): mean (SD)

| | | | | | |
|---|---|---|---|---|---|
| Baseline | 2.3648 (1.1530) | 2.0496 (1.0725) | | | |
| Day 28 | 1.6198 (0.5892) | 2.0720 (0.8096) | −0.553 | (−1.157; 0.050) | 0.070¹ |

Creatinine (mmol/24 hours): mean (SD)

| | | | | | |
|---|---|---|---|---|---|
| Baseline | 10.44 (5.10) | 8.95 (3.12) | | | |
| Day 28 | 9.68 (3.29) | 9.79 (2.97) | — | — | — |

Creatinine clearance (mL/min/1.73 m⁻²): median

| | | | | | |
|---|---|---|---|---|---|
| Baseline | 24.92 | 19.93 | | | |
| Day 28 | 23.78 | 15.81 | 1.12 | (0.83; 1.51) | 0.451¹ |

Urea (mmol/24 hours): mean (SD)

| | | | | | |
|---|---|---|---|---|---|
| Baseline | 343.53 (170.39) | 314.72 (167.45) | | | |
| Day 28 | 235.87 (88.51) | 280.92 (89.23) | — | — | — |

Urea clearance (mL/min/1.73 m⁻²): mean (SD)

| | | | | | |
|---|---|---|---|---|---|
| Baseline | 11.858 (5.820) | 11.771 (7.791 ) | | | |
| Day 28 | 11.244 (4.200) | 8.959 (3.261) | 2.28 | (−0.57; 5.13) | 0.111¹ |

GFR (ml/min/1.73 m⁻²): median

| | | | | | |
|---|---|---|---|---|---|
| Baseline | 19.37 | 18.10 | Placebo | (9.49; 20.22) | 0.777² |
| Day 28 | 18.23 | 13.54 | GHRH | (13.04; 24.81) | |

¹ANOVA for repeated measures, assuming normal distribution
²Treatment comparison with non-parametric ANCOVA

### Electrolytes

There were no statistically significant differences between the groups in 24-hour urinary electrolyte excretion (Table 33).

The mean difference between the 2 groups in changes from baseline to Day 28 in electrolyte excretion rate (EER) values was −0.002 mmol/kg/24 h for calcium; −0.136 mmol/kg/24 h for chloride; 0.020 mmol/kg/24 h for phosphate; −0.086 mmol/kg/24 h for potassium; and −0.020 mmol/kg/24 h for sodium.

The mean difference between the 2 groups in changes from baseline to Day 28 in electrolyte fractional excretion rate (EFER) values was −0.48% for calcium; −1.13% for chloride; −3.64% for phosphate; −7.93% for potassium; and 0.79 (ratio of means) for sodium.

The mean difference between the 2 groups in changes from baseline to Day 28 in fractional Pi reabsorption rate (Pi/GFR) was −0.00006 mmol/mL; for sodium excretion was −1.18 mmol/m²/24 h; for calcium excretion was −0.10 mmol/m²/24 h; and for calcium:creatinine ratio (ratio of means) was 0.82.

TABLE 33
24-h Urinary Electrolytes: ITT Population

| | | | | |
|---|---|---|---|---|
| | | Adjusted | | |
| GHRH | Placebo | difference or | | |
| N = 13 | N = 15 | ratio | 95% CI | P-value |

EER Calcium (mmol/kg/24 h): mean (SD)

| | | | | | |
|---|---|---|---|---|---|
| Baseline | 0.027 (0.027) | 0.017 (0.009) | | | |
| Day 28 | 0.036 (0.036) | 0.023 (0.016) | −0.002 | (−0.015; 0.010) | 0.688¹ |

EER Chloride (mmol/kg/24 h): mean (SD)

| | | | | | |
|---|---|---|---|---|---|
| Baseline | 2.213 (0.963) | 2.347 (1.598) | | | |
| Day 28 | 2.277 (1.028) | 2.223 (1.087) | −0.136 | (−0.885; 0.614) | 0.711¹ |

EER Phosphate (mmol/kg/24 h): mean (SD)

| | | | | | |
|---|---|---|---|---|---|
| Baseline | 0.328 (0.220) | 0.263 (0.095) | | | |
| Day 28 | 0.273 (0.129) | 0.236 (0.092) | 0.020 | (−0.055; 0.094) | 0.592¹ |

EER Potassium (mmol/kg/24 h): mean (SD)

| | | | | | |
|---|---|---|---|---|---|
| Baseline | 0.839 (0.332) | 0.650 (0.249) | | | |
| Day 28 | 0.656 (0.315) | 0.723 (0.278) | −0.086 | (−0.277; 0.105) | 0.359¹ |

EER Sodium (mmol/kg/24 h): mean (SD)

| | | | | | |
|---|---|---|---|---|---|
| Baseline | 2.428 (1.134) | 2.093 (1.191) | | | |
| Day 28 | 2.624 (1.020) | 2.535 (1.073) | −0.020 | (−0.767; 0.726) | 0.956¹ |

EFER Calcium (%): mean (SD)

| | | | | | |
|---|---|---|---|---|---|
| Baseline | 1.933 (1.164) | 2.054 (1.557) | | | |
| Day 28 | 2.743 (2.157) | 3.114 (2.915) | −0.48 | (−1.72; 0.76) | 0.430¹ |

EFER Chloride (%): mean (SD)

| | | | | | |
|---|---|---|---|---|---|
| Baseline | 3.636 (1.945) | 5.878 (4.352) | | | |
| Day 28 | 4.156 (2.541) | 5.599 (3.679) | −1.13 | (−3.59; 1.33) | 0.351¹ |

EFER Phosphate (%): mean (SD)

| | | | | | |
|---|---|---|---|---|---|
| Baseline | 35.882 (14.172) | 45.204 (12.124) | | | |
| Day 28 | 35.968 (12.979) | 40.472 (14.157) | −3.64 | (−15.00; 7.73) | 0.513¹ |

EFER Potassium (%): mean (SD)

| | | | | | |
|---|---|---|---|---|---|
| Baseline | 28.325 (13.618) | 34.332 (14.072) | | | |
| Day 28 | 26.766 (18.120) | 40.325 (18.804) | −7.93 | (−18.52; 2.67) | 0.135¹ |

EFER Sodium (%): mean (SD)

| | | | | | |
|---|---|---|---|---|---|
| Baseline | 3.068 (1.658) | 3.845 (2.468) | ratio | | |
| Day 28 | 3.359 (1.156) | 5.132 (3.609) | 0.79 | (0.52; 1.20) | 0.250³ |

Fractional Pi reabsorption rate Pi/GFR (mmol/ml): mean
(SD)

| | | | | | |
|---|---|---|---|---|---|
| Baseline | 0.861 (0.291) | 0.775 (0.305) | | | |
| Day 28 | 0.829 (0.211) | 0.873 (0.233) | −0.00006 | (−0.000; 0.000) | 0.4743¹ |

Calcium/Creatinine ratio: median

| | | | | | |
|---|---|---|---|---|---|
| Baseline | 0.145 | 0.088 | ratio | | 0.392³ |
| Day 28 | 0.195 | 0.123 | 0.82 | (0.51; 1.32) | |

Sodium excretion (mmol/m²/24 h): mean (SD)

| | | | | | |
|---|---|---|---|---|---|
| Baseline | 89.90 (42.10) | 76.76 (41.36) | | | |
| Day 28 | 96.70 (37.30) | 93.25 (38.34) | −1.18 | (−28.39; 26.02) | 0.929¹ |

Calcium excretion (mmol/m²/24 h): mean (SD)

| | | | | | |
|---|---|---|---|---|---|
| Baseline | 0.988 (0.916) | 0.606 (0.324) | | | |
| Day 28 | 1.295 (1.231) | 0.849 (0.584) | −0.10 | (−0.56; 0.36) | 0.667¹ |

¹ANOVA for repeated measures, assuming normal distribution
²Treatment comparison with non-parametric ANCOVA
³Treatment comparison with ANCOVA, assuming log-normal distribution

### Secondary Efficacy Variables: Dietary Assessment

There were no substantial changes from baseline or statistically significant differences between the groups in protein or energy intake (Table 34).

Mean protein intake was 62.5 g/day at baseline and 58.5 g/day on Day 28 for the GHRH(1-29)NH₂ synthetic analog group, and 62.0 g/day at baseline and 66.0 g/day on Day 28 for the placebo group. Mean energy intake was 1654.5 kcal/day at baseline and 1532.7 kcal/day on Day 28 for the GHRH(1-29)NH₂ synthetic analog group, and 1746.0 kcal/day at baseline and 1725.5 kcal/day on Day 28 for the placebo group.

TABLE 34
Dietary Assessments: ITT Population

| | | | | |
|---|---|---|---|---|
| GHRH | Placebo | Adjusted | | |
| N = 13 | N = 15 | difference | 95% CI | P-value¹ |

Protein intake (g/day): mean (SD)

| | | | | | |
|---|---|---|---|---|---|
| Baseline | 62.46 (17.71) | 62.01 (21.41) | | | |
| Day 28 | 58.49 (19.76) | 65.99 (21.71) | −12.2 | (−27.5; 3.0) | 0.110 |

Energy intake (kcal/day): mean (SD)

| | | | | | |
|---|---|---|---|---|---|
| Baseline | 1654.45 (394.75) | 1746.04 (548.61) | | | |
| Day 28 | 1532.70 (384.52) | 1725.52 (622.59) | −172.2 | (−450.3; 105.8) | 0.212 |

¹ANOVA for repeated measures, assuming normal distribution

### Efficacy Conclusions

Mean and median leucine oxidation was higher at Day 28 compared with baseline in the GHRH(1-29)NH₂ synthetic analog group but was lower or with very small change in the placebo group. The differences were not statistically significant when examined by parametric methods. However, statistically significant differences were detected between the groups in the ITT population (p=0.009) and in PP (Leucine Kinetic) population (p=0.040) using non-parametric methods.
There were no statistically significant differences between the GHRH(1-29)NH₂ synthetic analog group and placebo at Day 28 in mean leucine flux, mean protein synthesis, mean protein breakdown, median oxidation of flux, or median protein synthesis of flux. However, some non-significant differences were observed.
At Day 28, subjects were better nourished in the GHRH(1-29)NH₂ synthetic analog group than in the placebo group, and the difference between the groups was statistically significant (p=0.0083; non-parametric ANCOVA).
There were no statistically significant differences between the groups at Day 28 in any of the anthropometry measures, although some non-significant differences were observed.
Median fat mass (kg), measured by bioimpedance, was lower in the GHRH(1-29)NH₂ synthetic analog group at all visits and the differences were statistically significant at every visit except Day 14. The estimations of fat mass (%) and fat free mass (kg) were similar to fat mass. There were also statistically significant differences between the groups in extracelluar cell mass, extracellular water, intracellular water and total body water at Day 28.
From the DEXA scan results, there were statistically significant differences between the groups at Day 28 in mean fat mass (kg) (95% CI; 0.72-0.98, p=0.029), mean fat mass (%) (95% CI; 71%-96%, p=0.014) and mean fat free mass (kg) (95% CI; 0.5-5.4 kg, p=0.022).
A statistically significant difference was observed in fasting insulin and fasting cholesterol between groups. Median fasting insulin concentrations were consistently higher for the GHRH(1-29)NH₂ synthetic analog group compared with the placebo group (95% CI; CI: 1.131-2.829, p=0.015). Similarly, fasting cholesterol concentrations were higher for the GHRH(1-29)NH₂ synthetic analog group compared with the placebo group (95% CI; −1.02-−0.13, p=0.014). There were no statistically significant differences between the 2 groups in albumin, pre-albumin, transferrin, HbA_{1c}, fasting glucose, or fasting triglycerides.
On Day 28, the second component GH half-life was longer for the GHRH(1-29)NH₂ synthetic analog group compared with the placebo group (95% CI; 0.6-9.0, p=0.029). Statistically significant differences between the groups were also observed for the GHRH(1-29)NH₂ synthetic analog group compared with the placebo group in the following: time from onset to peak of GH burst was shorter (95% CI; −9.0-−0.1, p=0.048); basal GH secretion rate was greater (95% CI; 1.34-4.79, p=0.007); pulsatile GH secretion rate was greater (95% CI; 1.15-6.89, p=0.026); total GH secretion rate was greater (95% CI; 1.23-5.99, p=0.016); and mass per pulse was greater (95% CI; 7.2-45.8, p=0.009).
Median IGF-1 concentrations in the GHRH(1-29)NH₂ synthetic analog group were statistically significantly higher compared with those in the placebo group on every occasion of measurement. Mean IGFBP-1 concentrations in the placebo group were statistically significantly greater on Day 28 compared with the GHRH(1-29)NH₂ synthetic analog group (mean difference 28.40 ng/mL; 95% CI; −50.52-6.29; p=0.014). Differences between the 2 groups on Day 28 in IGFBP-3 concentrations were not statistically significantly different.
There were no statistically significant differences between the 2 groups in leptin concentrations (ratio of mean difference at Day 28 0.866 ng/mL) or in median leptin concentrations on any occasion of measurement.
There was little change in testosterone in either group. However, mean oestradiol values at Day 28 were almost half those at baseline in the GHRH(1-29)NH₂ synthetic analog group (30.400 pg/mL at baseline, 16.281 pg/mL at Day 28) compared with only a small decrease in placebo-treated subjects (31.983 pg/mL at baseline, 28.500 pg/mL at Day 28).
Median creatinine clearance at baseline was higher in the GHRH(1-29)NH₂ synthetic analog group (24.92 mL/min/1.73 m⁻²) compared with the placebo group (19.93 mL/min/1.73 m⁻²). Creatinine clearance was sustained at Day 28 in the GHRH(1-29)NH₂ synthetic analog group but was lower in the placebo group (23.78 mL/min/11.73 m⁻² GHRH(1-29)NH₂ synthetic analog group; 15.81 mL/min/11.73 m⁻² placebo group) but the difference was not statistically significant (95% CI; 0.83-1.51, p=0.451).
Mean urea clearance rate was sustained between baseline and Day 28 in the GHRH(1-29)NH₂ synthetic analog group (11.86 mL/min 1.73 m⁻² at baseline; 11.24 mL/min/1.73 m⁻² on Day 28) but fell in the placebo group (11.77 mL/min/1.73 m⁻² at baseline and 8.960 mL/min/1.73 m⁻² on Day 28), but the difference was not statistically significant (95% CI; −0.57-5.13, p=0.111).
Median GFR was sustained between baseline and Day 28 in the GHRH(1-29)NH₂ synthetic analog group (19.37 mL/min/1.73 m⁻² at baseline and 18.23 mL/min/1.73 m⁻² on Day 28) but fell in the placebo group (18.10 mL/min/1.73 m⁻² at baseline and 13.54 mL/min/1.73 m⁻² on Day 28), but the difference was not statistically significant (p=0.777).
There were no statistically significant differences between the groups in 24-hour urinary electrolyte excretion.
There were no substantial changes from baseline or statistically significant differences between the groups in protein or energy intake.

### Clinical Laboratory Evaluation

### Individual Clinically Significant Abnormalities

The number of subjects with abnormal and clinically significant laboratory values at baseline and at Day 28 is presented in Table 35 (haematology) and Table 36 clinical chemistry. Overall, the number of subjects with abnormal and clinically significant laboratory values was similar in the GHRH(1-29)NH₂ synthetic analog and placebo groups. No clear trends were observed.

Although the number of subjects with abnormal and clinically significant RBC and haemoglobin increased in 2 subjects between baseline and Day 28 in the GHRH(1-29)NH₂ synthetic analog group, there was the same number of subjects with these abnormal variables at Day 28 in the placebo group. However, the number of subjects with abnormal and clinically significant haematocrit rose from 1 (7.7%) subject at baseline to 4 (33.3%) subjects at Day 28 in the GHRH(1-29)NH₂ synthetic analog group but fell from 4 (26.7%) subjects at baseline to 2 (14.3%) subjects at Day 28 in the placebo group.

Every subject had abnormal and clinically significant creatinine and urea, which is consistent with chronic renal failure.

One subject in the GHRH(1-29)NH₂ synthetic analog group (Subject S18-27) had abnormal and clinically significant AST, ALT, GGT, urea, creatinine, phosphate, and potassium at Day 28. However, the number of subjects with abnormal and clinically significant potassium was lower at Day 28 than at baseline in both the GHRH(1-29)NH₂ synthetic analog and placebo groups.

The number of subjects with abnormal and clinically significant calcium rose from none at baseline to 3 (25.0%) subjects at Day 28 in the GHRH(1-29)NH₂ synthetic analog group but fell from 3 (20.0%) subjects at baseline to 1 (7.1%) subject at Day 28 in the placebo group. The number of subjects with abnormal and clinically significant phosphate increased by 1 between baseline and Day 28 in the GHRH(1-29)NH₂ synthetic analog group but fell by 1 in the placebo group, although there were 5 subjects in this category at baseline in the placebo group compared with 1 subject in the phosphate group.

TABLE 35
Number of Subjects With Abnormal and Clinically Significant
Haematology Abnormalities at Baseline and Day 28: Safety
Analysis Set

| | | GHRH | | Placebo | |
|---|---|---|---|---|---|
| | | N = 13 | | N = 15 | |
| Laboratory variable | Timepoint | n | (%) | n | (%) |
| RBC | Baseline | 1 | (7.7%) | 3 | (20.2%) |
| | Day 28 | 3 | (25.0%) | 3 | (21.4%) |
| Haemoglobin | Baseline | 3 | (23.1%) | 6 | (40.0%) |
| | Day 28 | 5 | (41.7%) | 5 | (35.7%) |
| Haematocrit | Baseline | 1 | (7.7%) | 4 | (26.7%) |
| | Day 28 | 4 | (33.3%) | 2 | (14.3%) |
| MCV | Baseline | 0 | (0.0%) | 0 | (0.0%) |
| | Day 28 | 0 | (0.0%) | 0 | (0.0%) |
| Platelet count | Baseline | 0 | (0.0%) | 0 | (0.0%) |
| | Day 28 | 0 | (0.0%) | 0 | (0.0%) |
| WBC | Baseline | 0 | (0.0%) | 0 | (0.0%) |
| | Day 28 | 0 | (0.0%) | 0 | (0.0%) |
| Neutrophils | Baseline | 1 | (7.7%) | 1 | (6.7%) |
| | Day 28 | 2 | (16.7%) | 3 | (21.4%) |
| Eosinophils | Baseline | 0 | (0.0%) | 0 | (0.0%) |
| | Day 28 | 0 | (0.0%) | 0 | (0.0%) |
| Basophils | Baseline | 0 | (0.0%) | 0 | (0.0%) |
| | Day 28 | 0 | (0.0%) | 0 | (0.0%) |
| Lymphocytes | Baseline | 0 | (0.0%) | 0 | (0.0%) |
| | Day 28 | 0 | (0.0%) | 2 | (14.3%) |
| Monocytes | Baseline | 1 | (7.7%) | 0 | (0.0%) |
| | Day 28 | 1 | (8.3%) | 0 | (0.0%) |

Percentages are based on the number of available data points

TABLE 36
Number of Subjects With Abnormal and Clinically Significant Clinical
Chemistry Abnormalities at Baseline and Day 28: Safety Analysis Set

| | | GHRH | | Placebo | |
|---|---|---|---|---|---|
| | | N = 13 | | N = 15 | |
| Laboratory variable | Timepoint | n | (%) | n | (%) |
| AST | Baseline | 0 | (0.0%) | 0 | (0.0%) |
| | Day 28 | 1 | (8.3%) | 0 | (0.0%) |
| ALT | Baseline | 0 | (0.0%) | 0 | (0.0%) |
| | Day 28 | 1 | (8.3%) | 0 | (0.0%) |
| GGT | Baseline | 0 | (0.0%) | 0 | (0.0%) |
| | Day 28 | 1 | (8.3%) | 0 | (0.0%) |
| Creatinine | Baseline | 13 | (100.0%) | 15 | (100.0%) |
| | Day 28 | 12 | (100.0%) | 14 | (100.0%) |
| Urea | Baseline | 13 | (100.0%) | 15 | (100.0%) |
| | Day 28 | 12 | (100.0%) | 14 | (100.0%) |
| Sodium | Baseline | 0 | (0.0%) | 0 | (0.0%) |
| | Day 28 | 0 | (0.0%) | 0 | (0.0%) |
| Potassium | Baseline | 4 | (30.8%) | 4 | (26.7%) |
| | Day 28 | 3 | (25.0%) | 2 | (14.3%) |
| Calcium | Baseline | 0 | (0.0%) | 3 | (20.0%) |
| | Day 28 | 3 | (25.0%) | 1 | (7.1%) |
| Chloride | Baseline | 0 | (0.0%) | 0 | (0.0%) |
| | Day 28 | 0 | (0.0%) | 0 | (0.0%) |
| Phosphate | Baseline | 1 | (7.7%) | 5 | (33.3%) |
| | Day 28 | 2 | (16.7%) | 4 | (28.6%) |

Percentages are based on the number of available data points

The number of subjects with abnormal and clinically significant bicarbonate rose from 2 (15.4%) subjects at baseline to 3 (25.0%) subjects at Day 28 in the GHRH(1-29)NH₂ synthetic analog group, and from 2 (13.3%) subjects at baseline to 4 (28.6%) subjects at Day 28 in the placebo group.

The number of subjects with normal, out of range but clinically not significant, and out of range and clinically significant is presented in Section 14, Tables 157 (haematology), 158 (clinical chemistry) and 159 (blood gas analysis).

### Vital Signs, Physical Findings and Other Observations Related to Safety

### Vital Signs

Mean systolic blood pressure was lower at Day 28 compared with baseline in the GHRH(1-29)NH₂ synthetic analog group but higher compared with baseline in the placebo group (Table 37). Mean systolic blood pressure in the GHRH(1-29)NH₂ synthetic analog group on other occasions of measurement was 142.0 mmHg at screening, 136.4 mmHg at Day 7, 139.9 mmHg at Day 14, and 143.1 mmHg at Day 21, suggesting that the low value at Day 28 was not part of a trend. Similarly, mean diastolic blood pressure was lower at Day 28 compared with baseline in the GHRH(1-29)NH₂ synthetic analog group but this was not part of a trend. Heart rate did not change between baseline and Day 28.

TABLE 37
Vital Signs at Baseline and Day 28: Safety Analysis Set

| | GHRH | Placebo |
|---|---|---|
| | N = 13 | N = 15 |
| | mean (SD) | mean (SD) |
| Systolic blood pressure | | |
| (mmHg) | | |
| Baseline | 144.8 (16.0) | 143.7 (20.2) |
| Day 28 | 128.3 (16.5) | 148.2 (16.4) |
| Diastolic blood pressure | | |
| (mmHg) | | |
| Baseline | 82.7 (10.9) | 82.2 (11.5) |
| Day 28 | 76.7 (8.1) | 79.0 (11.9) |
| Heart rate (bpm) | | |
| Baseline | 77.7 (11.1) | 69.4 (8.6) |
| Day 28 | 76.4 (9.8) | 68.9 (11.8) |

### Physical Examination

The number of subjects with abnormal findings in physical examination at baseline and at Day 28 is summarized in Table 38. There were no body systems in which there was a trend towards change in physical examination. Any physical examination findings after the screening visit were reported as AEs.

TABLE 38
Number of Subjects With Abnormal Findings on Physical
Examination at Baseline and Day 28: Safety Analysis Set

| | | GHRH | | Placebo | |
|---|---|---|---|---|---|
| | | N = 13 | | N = 15 | |
| Body system | Timepoint | n | (%) | n | (%) |
| Cardiovascular | Baseline | 6 | (46.2%) | 8 | (53.3%) |
| | Day 28 | 3 | (25.0%) | 7 | (50.0%) |
| Central nervous system | Baseline | 0 | (0.0%) | 0 | (0.0%) |
| | Day 28 | 0 | (0.0%) | 0 | (0.0%) |
| Ear | Baseline | 2 | (15.4%) | 1 | (6.7%) |
| | Day 28 | 2 | (16.7%) | 1 | (7.1%) |
| Nose and throat | Baseline | 5 | (38.5%) | 2 | (13.3%) |
| | Day 28 | 5 | (41.7%) | 3 | (21.4%) |
| Gastrointestinal | Baseline | 4 | (30.8%) | 1 | (6.7%) |
| | Day 28 | 5 | (41.7%) | 1 | (7.1%) |
| Genitourinary | Baseline | 3 | (23.1%) | 4 | (26.7%) |
| | Day 28 | 2 | (16.7%) | 4 | (28.6%) |
| Musculoskeletal | Baseline | 7 | (53.8%) | 9 | (60.0%) |
| | Day 28 | 6 | (50.0%) | 8 | (57.1%) |
| Respiratory | Baseline | 0 | (0.0%) | 1 | (6.7%) |
| | Day 28 | 1 | (8.3%) | 2 | (14.3%) |
| Skin | Baseline | 9 | (69.2%) | 8 | (53.3%) |
| | Day 28 | 9 | (75.0%) | 9 | (64.3%) |
| Other | Baseline | 3 | (23.1%) | 2 | (14.3%) |
| | Day 28 | 5 | (41.7%) | 2 | (14.3%) |

Percentages are based on the number of available data points

### DISCUSSION AND OVERALL CONCLUSIONS

Malnutrition in CRF has a complex aetiology, including a reduced nutrient intake, increased oxidative stress, chronic inflammation, and metabolic and endocrine alterations.

The assessment of nutritional status in CRF patients, according to the National Kidney Foundation/Dialysis Outcome Quality Initiative Guidelines, should be made by integrating clinical, biochemical and anthropometric measurements. Among the anthropometric measurements, mid-arm muscle circumference, skinfold thickness and hand-grip strength are frequently performed. Through a combination of all these data, one obtains the Subjective Global Nutritional Assessment. Among the biochemical biomarkers, serum albumin and prealbumin have been proved to be useful indicators of morbidity. As immune function is impaired in CRF, several immune parameters are frequently evaluated to support conventional measures of nutritional status.

Body composition can be estimated using various approaches such as bioimpedance analysis, DEXA, or, in experimental settings, whole body K measurement, in vivo neutron activation, IR reactance, computerized tomography and nuclear magnetic resonance.

The overall intention of this study was to evaluate whether administration of the GHRH(1-29)NH₂ synthetic analog, a novel GHRH superagonist, into malnourished patients with CKD, presumed to be resistant to the action of the endogenous GH, will stimulate the secretion of endogenous GH in a natural pulsatile way, that will in turn have beneficial effect on the net balance of circulating GH and IGF binding proteins that will result in an increase in circulating free IGF-1 and protein anabolism. The working hypothesis was that malnourished patients with CKD are catabolic and that leucine oxidation to irreversibly lost CO₂ is higher than in normal age-matched healthy population resulting in muscle wasting. We hypothesized, that like in the case of GH replacement therapy, the GHRH(1-29)NH₂ synthetic analog will induce positive changes in body composition and nutritional status without side effects accompanying exogenous rhGH administration. We did not expect that short duration of treatment will have an effect on muscle function so we did not perform a grip test. Additionally, based on unpublished animal data, we hypothesized that through direct binding to GHRH receptor in kidneys, the GHRH(1-29)NH₂ synthetic analog will slow down the progression of kidney function damage by protecting renal tissue against oxidative stress.

This was a double-blind, placebo-controlled study that was designed to show superiority of the GHRH(1-29)NH₂ synthetic analog over placebo on protein turnover as assessed by leucine oxidation rate. The planned sample size was for 16 subjects per group to be randomized. However, recruitment was challenging and the study was stopped with 13 subjects randomized to the GHRH(1-29)NH₂ synthetic analog group and 15 subjects randomised to the Placebo group. Two subjects, 1 from each group, did not complete the study. Therefore, full data sets were available for only 12 subjects treated with the GHRH(1-29)NH₂ synthetic analog and 14 subjects treated with placebo.

There was much scope for variability in this study. CKD is a progressive disorder and although subjects for this study were eligible only if they had relatively stable disease, some disease progression was still likely. Furthermore, the efficacy of a treatment has to be measurable against a background of worsening disease. In addition, the release of endogenous GH is not consistent throughout the day. Therefore, the primary measure of efficacy had to be sufficiently sensitive and consistent to overcome these confounding factors.

### Study Primary Objective

Since based on published clinical data we did not expect the GHRH(1-29)NH₂ synthetic analog to have significant effect on muscle mass increase in one month of treatment, we selected measurement of protein turnover, assessed by ¹³C-leucine kinetics, as the primary objective although total body protein turnover is not a clinically relevant or recognized endpoint for demonstration of a compound efficacy in the treatment of malnutrition or wasting disease. ¹³C-leucine kinetic studies have been used successfully in several clinical studies demonstrating reduction of leucine oxidation in patients with CF, CRF, sepsis, or burns following therapy with human recombinant GH.

Thus, we expected subjects in our study to be catabolic and have high oxidation rates at baseline that could be decreased by the administration of the GHRH(1-29)NH₂ synthetic analog for 28 days.

However, leucine oxidation rate at baseline was found to be very low (GHRH: 7.74 μmol/kg/hr, Placebo: 9.83 μmol/kg/hr) and even lower than that reported for normal age matched healthy adults and thus not indicative of catabolism. It has been shown that in severely burned patients, leucine flux values were 222 μmol/kg/hr and oxidation rates about 49 μmol/kg/hr (26). Those are the values for very strong catabolic levels. In chronic renal patients, it has been found that leucine flux is 95 μmol/kg/hr and healthy controls about 92 μmol/kg/hr. The leucine oxidation rates for CHF were 12-14 μmol/kg/hr and 10-14 μmol/kg/hr in healthy controls. Much higher values of whole-body protein turnover were reported in HIV infection, acute sepsis, burns, trauma, or malignancy. The flux levels we found in our study were similar but the oxidation, protein synthesis, and protein breakdown rates were still much lower than in other catabolic states.

It is also possible that dose and duration of GHRH treatment may have had an effect on leucine oxydation. In adult subjects with GH deficiency, exogenous GH-induced changes in protein metabolism were influenced by the dose and duration of GH treatment. Suppression of protein oxidation occurred soon after initiation of GH treatment in the higher dose (6 μg/kg/d) group and predicted a later gain in LBM. The maximal changes in leucine oxidation (−3.9±1.1 versus +0.8±1.8 μmol/min, p=0.03) were significantly greater in the higher, than the low dose (3 μg/kg/d) group. The acute reduction in leucine oxidation at two weeks in the higher dose group was no longer significant after 12 weeks. The change in leucine oxidation after two (r=−0.53, p=0.035), but not 12, weeks was significantly correlated with the change in LBM.

In this study, mean and median leucine oxidation was higher at Day 28 compared with baseline in the GHRH(1-29)NH₂ synthetic analog group but was lower or with very small change in the placebo group. For the ITT population, mean leucine oxidation was 7.74 μmol/kg.hr at baseline and increased by 2.38 μmol/kg.hr at Day 28 in the GHRH(1-29)NH₂ synthetic analog group, whereas in the placebo group, baseline leucine oxidation was higher than in the GHRH(1-29)NH₂ synthetic analog group at 9.83 μmol/kg.hr, and rose by just 0.84 μmol/kg.hr at Day 28. Although the adjusted mean difference GHRH(1-29)NH₂ synthetic analog-placebo was 1.1 μmol/kg.hr, the 95% CI was wide (−6.0; 8.2) and the difference between the groups was not statistically significant (p=0.750).

However, the assumption of normality was questionable and median values showed a different pattern from the mean values. For the ITT population, median leucine oxidation was 5.8 μmol/kg.hr at baseline and increased by 2.3 μmol/kg.hr at Day 28 in the GHRH(1-29)NH₂ synthetic analog group. In the placebo group, baseline leucine oxidation was, again, higher than in the GHRH(1-29)NH₂ synthetic analog group at 8.8 μmol/kg.hr, and fell by 1.3 μmol/kg.hr at Day 28. When a non-parametric analysis method was applied to the data, statistically significant differences were detected between the groups in both the ITT population (p=0.009) and PP (Leucine Kinetic) populations (p=0.040). Therefore, a statistically significant effect of the GHRH(1-29)NH₂ synthetic analog has been demonstrated on the basis that the data are not normally distributed.

The results of other leucine kinetic data (protein synthesis, protein breakdown, leucine flux) normalized for body weight and FFM also did not demonstrate any statistically significant differences between treatments on day 28. The statistical analysis of the results in the per-protocol population was in-line with the intent-to-treat analysis.

It remains unexplained why leucine oxidation rates were low in this study. Maybe the oxidation rate was high initially at the start of the illness but decreased to preserve protein breakdown or maybe the normal protein turnover is the result of the control of metabolic acidosis. Acidotic conditions stimulate muscle proteolysis via the ubiquitin-proteasome pathway and lead to enhanced oxidation of branched-chain amino acids. Correction of metabolic acidosis due to renal failure in humans reduces whole body proteolysis.

A variety of different catabolic factors may determine whether CKD patients become cachectic. The precise role for each one of these factors as well as the intracellular pathways activated in muscle as a result of disease are unknown and remain to be defined.

### Study Secondary Objectives

The secondary objectives were to determine the effect of the GHRH(1-29)NH₂ synthetic analog on endogenous 24-hour growth hormone (GH) secretion, circulating total insulin-like growth factor (IGF-1) and its binding proteins IGFBP-1 and IGFBP-3, fat-free mass (FFM) and fat mass (FM) as assessed by Dual X-ray absorptiometry (DEXA), bioimpedance (BIA) and conventional anthropometry, biochemical parameters of nutritional and metabolic states (serum albumin, pre-albumin, transferrin, leptin, adiponectin, glycosylated haemoglobin (HbA1c), fasting glucose, insulin, and lipids), spontaneous nutrient intake, and safety and tolerability.

The GH/IGF-I axis in CKD is changed markedly compared with the normal axis, as illustrated in. In CKD, the circulating levels of GH are not reduced and maybe even increased while circulating levels IGF-I are generally decreased. This decreased GH production is a consequence of attenuated GHRH expression and increased somatostatin release. Acidosis may also suppress GH secretion. The metabolic clearance rate of GH is obviously decreased in advanced CKD.

Furthermore, there is reduced effectiveness of endogenous GH and IGF-I, which probably plays a major role in reducing linear bone growth. The reduced effectiveness of endogenous IGF-I is likely due to decreased levels of free, bioactive IGF-I as levels of circulating inhibitory IGFBP1, 2, 4 and 6 are increased. In addition, less IGF-I is circulating in the complex with ALS and IGFBP3 as the result of increased proteolysis of IGFBP3. In sum, these lead to decreased IGF-I receptor activation and a decreased feedback to the hypothalamus and pituitary. Low free IGF-I and high IGFBP levels probably contribute to a reduced renal function and lead to a reduced stature. The direct effects of GH on bone, which are not fully understood, also are blunted.

### Endogenous 24-Hour Growth Hormone (GH) Secretion

For ITT population, the median amount of GH secreted (AUC μIU/ml×min) during the first 4 hours after morning treatment administration was at baseline 315 for the GHRH(1-29)NH₂ synthetic analog and 278 for placebo while on day 28^{th} it was 3891 for GHRH and 299 for placebo, increase of 1137% for the GHRH(1-29)NH₂ synthetic analog and 30% for placebo. The mean AUC was statistically significant (p<0.0001) higher in the GHRH(1-29)NH₂ synthetic analog group as compared placebo, ratio of means was 9.5 higher in GHRH group (95% C from 4.4 to 20.5). The mean concentration of GH secreted (μIU/ml) during the first 4 hours after morning treatment administration was at baseline 1.72 for the GHRH(1-29)NH₂ synthetic analog and 1.68 for placebo while on day 28^{th} it was 21.26 the GHRH(1-29)NH₂ synthetic analog and 2.19 for placebo, statistically significant (p<0.0001) increase of 1136% for the GHRH(1-29)NH₂ synthetic analog and 30% for placebo.

The mean amount of GH secreted (AUC μIU/ml×min) in 24 hours following treatment administration was at baseline 3770.62 for the GHRH(1-29)NH₂ synthetic analog and 3388.80 for placebo while on day 28^{th} it was 18579.9 for the GHRH(1-29)NH₂ synthetic analog and 4146.36 for placebo, increase of 393% for the GHRH(1-29)NH₂ synthetic analog and 22% for placebo. There was a statistically significant difference (p=0.00025) between means at day 28, the mean AUC was 11585 μIU/ml×min higher in the GHRH(1-29)NH₂ synthetic analog group (95% CI from 4571 to 18599 μIU/ml×min)

This increase clearly demonstrates that the GHRH(1-29)NH₂ synthetic analog can stimulate GH secretion in patients with CKD and that this amount secreted daily corresponds to the amount of circulating GH following supplementation with recombinant human GH at a dose of 0.25 mg/kg given once for approximately a 1-month period reported in the literature for adults with GH deficiency. Mean concentration of GH at baseline in our CKD patient population (2.49 for GHRH and 2.26 for placebo arm, μIU/ml) was the same as the rate reported in literature but lower than in healthy age-matched subjects (male: 2.64±1.26 μIU/m, female: 3.93±2.61 μIU/ml).

Mean basal GH secretion rate at baseline in our CKD patient population was 26.9 for the GHRH(1-29)NH₂ synthetic analog and 19.7 for placebo arm (mU/L/24 h).

The mean number of GH pulses at baseline, mean pulse frequency per 24 hours, inter-pulse regularity, or % pulsatile secretion rate did not change following 28 days of treatment with either GHRH or placebo (p=0.82, p=0.58, p=0.88, p=0.95) indicating that the GHRH(1-29)NH₂ synthetic analog treatment did not effect the natural pulsatile rhythm of endogenous GH secretion.

### Total IGF-1 and its Binding Proteins

In the ITT population, there was statistically significant difference between treatment groups in median total IGF-1 (ng/ml) on day 7 (p=0.0006), 14 (p=0.0012), 21 (p<0.0001) and on day 28 (p<0.0001). The median IGF-1 was higher in the GHRH(1-29)NH₂ synthetic analog group at all visits. At baseline, total median IGF-1 was 335.8 in the GHRH(1-29)NH₂ synthetic analog group and 384.8 in placebo group while on day 28 it was 684.2 in the GHRH(1-29)NH₂ synthetic analog group (104% increase) and 398.3 in placebo group (3.5% increase). Median IGF-1 returned to baseline value after treatment discontinuation, day 42 (368.2 in the GHRH(1-29)NH₂ synthetic analog and 343.2 in placebo). The median baseline total circulating IGF-1 in our patient population with CKD was not however lower than the level reported in the literature for these type of patients nor lower than in the age-matched healthy adults (39,40,41). This confirms our starting hypothesis that malnutrition (protein wasting) in CKD is the result of the resistance to the endogenous circulating GH and IGF-1 rather than the lack of it. It is also possible that the circulating IGF-1 is bound to IGFBP-1 and thus is not available for its action.

Most circulating IGF-1 is produced by the liver in response to GH and mediates many of the anabolic actions of GH. As expected, we have demonstrated that increase in circulating GH stimulated in turn the secretion of IGF-1.

Availability of IGF-1 for its action is controlled by IGFBP-1, IGFBP-3 and acid-labile subunit. Since we were not able to determine free IGF-1, we have measured circulating IGFBP-1 and IGFBP-3. IGFBP-1, which is also derived from the liver, binds to free IGF-1 and reduces free IGF-1 availability and its action. Elevated IGFBP-1 levels are observed in patients with metabolic disturbances associated with liver disease. IGF-1 circulates as a ternary complex with IGFBP-3 and the acid-labile subunit, and both liver-derived proteins are under the control of GH. IGFBP-3 stabilizes circulating IGF-1, and reductions in IGFBP-3 can contribute to a decrease in IGF-1 levels (due to decreased stability of the complex). Thus, increase in serum concentrations of IGFBP-3 should accompany an increase in IGF-1.

In our study, there was statistically significant difference between the GHRH(1-29)NH₂ synthetic analog and placebo groups on mean IGFBP-1 (ng/ml) post baseline (p=0.0139). The mean difference between GHRH and placebo arm was −23.89 ng/ml (95% CI from −42.44 ng/ml to −5.33 ng/ml). The decrease in IGFBP-1 level corresponded well with the increase in IGFBP-3 level with an overall mean difference between treatments of 452.45 ng/ml (95% CI from −150.81 ng/ml to 1055.71 ng/ml).

### Body Composition: Fat-Free Mass (FFM), Fat Mass (FM), Body Cell Mass, Intracellular and Extracellular Water, Basal Metabolic Rate, Mineral Bone Density:

The efficacy of nutritional treatment was evaluated on the basis of changes in the body components, an important diagnostic tool in the assessment of health and nutritional status. The early stage of malnutrition is characterized by decreased body cell mass and increased extracellular water with unchanged net fat free mass.

Body composition was measured by DEXA, bioimpedance (bioelectrical impedance, BIA) and standard anthropometry.

There was a statistically significant difference between treatment groups on mean body weight (p=0.019). The subjects in the GHRH(1-29)NH₂ synthetic analog group were on average 0.96 kg (95% CI from 0.17 to 1.75 kg) heavier than the subjects in placebo group after baseline, difference attributable mainly to the increase in FFM.

Bioelectrical impedance (BIA) was used to evaluate body water content (total body water %, extracellular water and intracellular water), fat free mass and fat mass using Maltron BioScan 916 and BIA 450 apparatus from Biodynamics Corp. The method is fast, precise and noninvasive.

Bioimpedance measurements were done at every visit from baseline (day 0) on days 7, 14, 21 to 28. Weight and FFM (kg) were measured and FM (kg, %) and FFM (%) were derived. There was an increase from baseline in median FFM (3.3 kg) and decrease in median FM (1.8 kg) in all except one (11/12) patients in the GHRH(1-29)NH₂ synthetic analog group on day 28 while there was a decrease in median FFM (0.8 kg) and increase in median FM (1.3 kg) in the placebo arm.

Difference between treatment groups in median FM (kg) on day 7 (p=0.037), 21 (p=0.0013) and 28 (p=0.0081) but not on day 14 (p=0.11) was statistically significant. The median FM (kg) was lower in the GHRH(1-29)NH₂ synthetic analog group at all visits after baseline (day 0: 16, day 7: 13.3, day 14: 13.4, day 21: 12.6, and day 28: 12.2). The median FM (kg) was statistically significantly higher in placebo group at all visits after baseline except 4 (day 0: 14.1, day 7: 14.7, day 14: 13.9, day 21: 15.2, and day 28: 16.5). The results of the FM (%) were similar to FM (kg).

The shown decrease in FM is most probably due to the increase in circulating GH level since GH directly promotes lipolysis which results in the reduction of adipose tissue (body fat) and increased basal metabolic rate (see section below).

The results of the FFM (kg & %) were similar to FM, i.e. statistically significant difference was detected at all other visits but visit 4 (day 14) and the median FFM (%) and FFM (kg) was higher in the GHRH(1-29)NH₂ synthetic analog group at all visits after baseline. The median FFM (kg) in the GHRH(1-29)NH₂ synthetic analog group was on day 0: 48.8, day 7: 50.4, day 14: 50.8, day 21: 50.1, and day 28: 51.1). The median FFM (kg) in placebo group at all visits was on day 0: 51.7, day 7: 51.3, day 14: 52, day 21: 51, and day 28: 52. The differences in FM and FFM between groups were still maintained on Day 42 post baseline.

The shown increase in lean body mass (or FFM) is most likely mediated by the significantly elevated circulating GH and IGF-1 and attributable to the observed increase in body cell mass (BCM), extracellular cell mass ECM), and intra-(ICW) and extra-cellular water (ECW) measured by bioimpedance. It is a well documented fact that GH increases muscle mass through the creation of new muscle cells and also increases water content in the FFM. The lean body mass is composed of the BCM and ECM. In our study both were increased in the GHRH group.

Although there was no statistically significant difference between treatments on mean body cell mass at day 28 (p=0.076) there was an increase in BCM from baseline at each study visit and on day 28^{th} of treatment with GHRH but not with placebo. The increase from baseline was in all but one patient at all visits and ranged from median 1.3 kg on day 7 to 1.8 kg on day 28 while median BCM decreased from baseline by 0.6 kg on day 28 in the placebo group. The median BCM (kg) in GHRH group was on day 0: 23, day 7: 25.1, day 14: 24.3, day 21: 23.2, and day 28: 22.6 and in placebo group 25 on day 0, 26.1 on day 7, 27.5 on day 14, 25.4 on day 21 and 24.6 on day 28.

BCM constitutes the metabolically active compartment of the human body. The increase in BCM has been reported to correlate positively with the decrease in wasting and an increase in muscle strength, regain of function and reduction in hospital stay in a variety of muscle wasting conditions such as AIDS, COPD. Depletion of BCM is on the other hand strongly associated with disease progression and death in human immunodeficiency virus (HIV)-infected patients. BCM decreases in the course of natural aging processes, primarily due to a loss in skeletal muscle mass.

There was statistically significant difference between treatments on median extra cellular cell mass (ECM) to day 28 (p=0.0043) and there was an increase in ECM from baseline at each study visit and on day 28^{th} of treatment with the GHRH(1-29)NH₂ synthetic analog but not with placebo. The increase from baseline was in all patients at all visits and the median was 0.8 kg on day 7 to 1.7 kg on day 28 while median ECM decreased from baseline by 0.3 kg on day 28 in placebo group. The median ECM (kg) in the GHRH(1-29)NH₂ synthetic analog group was on day 0: 23.5, day 7: 24.4, day 14: 25.4, day 21: 25.9, and day 28: 24.7 and in placebo group 25.7 on day 0, 23.9 on day 7, 24.3 on day 14, 23.8 on day 21 and 23.6 on day 28.

Correct water content is essential for the maintenance of homeostasis and is important for temperature regulation, digestion, absorption of nutrients, elimination of toxins, joints function and neuron transmission.

There was statistically significant difference between treatments on ECW and total water content at day 28 (p=0.0003, p=0.017). Treatment with the GHRH(1-29)NH₂ synthetic analog but not with placebo increased in all patients median ECW by 1.9 L, ICW by 1.2 L and total body water by 3 L from baseline on day 28 and gradually also at all visits. However, the ratio of median ECW/ICW (1.2) on day 28 (the GHRH(1-29)NH₂ synthetic analog 19.1/15.8 L vs. 17.5/18.0 L placebo) did not change considerably from that at baseline for the GHRH(1-29)NH₂ synthetic analog group (1.04) (GHRH 17.7/17.0 L vs. 18.1/18.2 L placebo). ECW increase is a common negative side effect of GH treatment and oedema is the most common sign of ECW expansion. In our case, however, an increase in total water was not associated with an increase in blood pressure (see below). ICW increase is however desirable and the ratio of ECW to ICW is an important indicator of morbidity/mortality in many diseases and its change has very important clinical implications. In patients on peritoneal dialysis (PD) for every increase of 0.1 in the E/I value, the relative risk of death was 1.368.

Reduction in ICW is often associated with Protein-Energy Malnutrition state and is indicative of reduced protein reserve. Restoration of ICW is clinically important in the treatment of malnutrition.

Treatment with the GHRH(1-29)NH₂ synthetic analog has significantly increased basal metabolic rate (BMR) as measured by BIA. Treatment with the GHRH(1-29)NH₂ synthetic analog increased median BMR by 49 kcal from baseline on day 28 while placebo decreased BMR by 10 kcal. The same trend was observed at all visits.

Increase in BMR helps to lose weight by burning fat. While exercise increases BMR, its effect is short lasting. The most effective way to increase BMR is an increase in LBM. Each pound of LBM can burn 35 to 50 calories per day, whereas a pound of fat only burns 2 to 3 calories per day.

Thus, observed in our study increase in GH, LBM and BMR may explain the reduction in FM.

The above observed effects of GHRH treatment on FFM and FM were further confirmed by the results of DEXA measurements.

All subjects had a whole body DEXA scan at baseline and at day 28. Mean body composition values (total tissue, fat mass, lean tissue and bone mineral content) were recorded for the total body and defined regional areas (arms, legs, trunk) if possible. FFM was derived for total body and for regional areas.

There was statistically significant difference between treatment groups on mean total FM (kg) at day 28 (p=0.029). Patients in the GHRH(1-29)NH₂ synthetic analog arm lost mean 0.51 kg while those in placebo arm gained mean 1.21 kg. The mean FM in the GHRH(1-29)NH₂ synthetic analog group was 84% of the mean in placebo group (95% CI from 72% to 98%). Similar result was detected in FM (%) at day 28 (p=0.014). The mean FM (%) in the GHRH(1-29)NH₂ synthetic analog group was 83% of the mean in placebo group (95% CI from 71% to 96%).

A decrease in FM was most pronounced in the arm and trunk regions. While patients in the GHRH(1-29)NH₂ synthetic analog group lost on average 0.22 kg of FM in arms and 0.30 kg in trunk, patients in placebo arm gained 0.32 kg in arms and 0.44 kg in trunk. The direction of the difference indicated a beneficial effect of the GHRH(1-29)NH₂ synthetic analog on fat distribution.

DEXA scans also showed that bone mineral content in the trunk tended to increase in the GHRH(1-29)NH₂ synthetic analog group whereas there was no change in the placebo group, suggesting that the subjects were starting to recover some loss of bone mineral density.

Also in FFM (kg) statistical significant difference was detected (p=0.022). The mean FFM (kg) was 2.9 kg higher in the GHRH(1-29)NH₂ synthetic analog group than in placebo group (95% CI from 0.5 to 5.4 kg). The increase in FFM was most pronounced in the leg and trunk regions. While patients in the GHRH(1-29)NH₂ synthetic analog group gained mean 1.83 kg FFM as total body, 0.15 kg was gained in arms, 1.03 kg in legs and 0.59 kg in trunk, patients in placebo arm lost 1.39 kg FFM as total body, 0.11 kg in arms, 0.78 kg in legs, and 0.44 kg in trunk.

These results however have to be interpreted with caution as DEXA does not differentiate between muscle and water gain.

These effects by DEXA corroborate BIA results making the effect stronger, which is further supported by no difference in calorie/protein intake between groups suggesting better utilization of nutrients in the GHRH group.

There were no statistically significant differences between the groups at Day 28 in any of the anthropometry measures. However, none were expected because the study was not powered for statistical significance in anthropometry. There were no clear differences between the groups in terms of median fat mass and mean fat free mass, but some small differences were observed in biceps, triceps, and suprailiacal skinfold thicknesses.

### Subjective Global Assessment

At Day 28, subjects were better nourished in the GHRH(1-29)NH₂ synthetic analog group than in the placebo group and the difference was statistically significant (p=0.0083; non-parametric ANCOVA). Out of 9 mild to moderate malnourished patients in the GHRH group 6 become well nourished after 28 days of therapy while all 9 mild to moderate malnourished patients in the placebo group remained malnourished. The finding suggests that the subjects benefited from treatment with the GHRH(1-29)NH₂ synthetic analog.

### Biochemical Parameters of Nutritional State: Blood Albumin, Pre-Albumin, and Transferring

There were no statistically significant differences between treatments on mean serum albumin (p=0.43), pre-albumin (p−0.076), and transferrin (p=0.32) at day 28 or between baseline and day 28^{th} of treatment with either the GHRH(1-29)NH₂ synthetic analog or placebo.

CKD is frequently associated with a complex of malnutrition, inflammation and accelerated atherosclerosis (the MIA syndrome), leading to excessive morbidity and mortality even before end-stage renal disease is attained. Inflammation is associated with malnutrition and atherosclerosis in ESRD. Although various factors associated with the dialysis procedure, such as bio-incompatibility and nutrient losses, may contribute to malnutrition, recent studies have shown that malnutrition is common even before the start of renal replacement therapy. A low serum albumin (S-albumin) level has been used as a marker for malnutrition for many years and is considered to be an important risk factor for mortality. However, S-albumin may not be a valid nutritional marker as it is affected by inflammation and external losses. As both inflammation and inadequate nutritional intake can decrease the concentration of S-albumin, much of the previously reported relationship between S-albumin, malnutrition, and mortality in patients undergoing RRT may be due to an inflammatory process rather than poor nutritional intake. Indeed, inflammatory markers, such as CRP and IL-6, are strong predictors of both poor outcome and malnutrition in patients on dialysis. Several factors, such as altered protein synthesis, overhydration, reduced protein intake, bowel malabsorption and protein losses (as during nephrotic syndrome) influence plasma albumin concentration, a lowering of which is usually considered a late marker of undernutrition. A fall in prealbumin is an earlier indicator that correlates with body weight, mean arm circumference, creatinine and albumin concentration but it is not totally reliable for it is commonly excreted by the kidney. Transferrin, with a half-life of 7-8 d, is very sensitive to various dietary and non-dietary factors. Serum transferrin rises in Fe deficiency, whilst its decrease indicates Fe overload or inflammation.

### Hormone Levels: Testosterone and Oestradiol

Testosterone and oestradiol were measured at baseline and Day 28 because these can be affected by GH and can lead to secondary changes in body composition. Oestradiol values were almost halved at Day 28 compared with baseline in the GHRH(1-29)NH₂ synthetic analog group whereas there was little change in the placebo group. There was little change in mean testosterone values in either group, although the variability in the data was wide.

### Fat Metabolism: Fasting Triglycerides, Fasting Cholesterol, Leptin and Adiponectin

There was no statistically significant difference between treatments on mean triglycerides at day 7, 14, and 28 (p=0.11). There was, however, statistically significant difference between treatments on mean cholesterol at day 7, 14 and 28 (p=0.014). Mean difference (GHRH-Placebo) was −0.58 (95% CI: −1.02, −0.13). The mean cholesterol (mmol/l) was reduced by up to 10% in almost all patients in the GHRH(1-29)NH₂ synthetic analog group (10/12).

Although there was no statistically significant difference between treatments on mean leptin post baseline (p=0.69) and adiponectin at day 28 (p=0.78), there was a substantial reduction in mean leptin from baseline on day 28^{th} of treatment with the GHRH(1-29)NH₂ synthetic analog but not with placebo. The mean leptin in the GHRH(1-29)NH₂ synthetic analog group was 70% of the mean in placebo group. The reduction was in all but two patients and ranged from 10-70% from baseline. Leptin decrease is positively correlated with fat reduction while increase in adiponectin is expected in fat reduction. The serum leptin level is frequently elevated in patients with CKD and associated with a loss of lean body mass. Increased plasma leptin concentration (as is observed in CRF) inhibits insulin production, thus favoring protein catabolism and leading to a worsening nutritional status. TNF-α stimulates preformed leptin release from adipocytes while a direct relationship between leptin levels, BMI, total body fat content, insulinaemia and TNF-a concentration has been widely demonstrated. Plasma leptin is inversely related to glomerular filtration rate (GFR) in patients with different levels of renal disease, thus suggesting that its removal is impaired from the early stages of CRF. In dialysed patients, as GFR decreases to 10.5 mL/min, leptin removal is not sufficient to effectively counterbalance its production, especially in women.

### Carbohydrate Metabolism: Glycosylated Haemoglobin (HbA1c), Fasting Glucose, Insulin

There were no statistically significant differences between treatments on day 28 on mean HbA1c (p=0.16) and fasting glucose at day 7, 14, and 28 (p=0.54). There was however, a statistically significant difference between treatments on day 7, 14, and 28 on mean fasting insulin (p=0.015). In the GHRH arm an increase in median was seen, median (μunits/ml) at day 7 was 14.2, at day 14 15.5 and at day 28 12.1. In placebo arm the median stayed approximately on the baseline level, median (μunits/ml) at day 3 was 5.4, at day 14 5.1 and at day 28 7.0. The adjusted ratio of means was 1.79 (95% CI from 1.13 to 2.83). GHRH analog treatment induced a significant increase in fasting insulin levels, which was not associated with adverse changes in fasting glucose or glycosylated hemoglobin.

Kidney Function Assessment: Urine Biochemistry Data: nPNA, Creatinine Clearance, Urea Clearance, GFR, EER, Fractional Pi Reabsorption Rate, and Sodium and Calcium Secretion

There was no statistically significant difference between treatments on mean nPNA at day 28 (p=0.07). The adjusted mean difference between GHRH and placebo arm was −0.553 g/kg body weight/24 h (95% CI −1.157 g/kg body weight/24 h to 0.050 g/kg body weight/24 h). nPNA is a valid and useful method for estimating protein intake. It is used as one of several independent measures to evaluate nutritional status of a patient. Although there was a decrease from mean value of 2.3648 g/kg body weight/24 h to 1.6198 g/kg body weight/24 h in GHRH arm, subjects still met the goals of nutrition intervention in CKD with level greater than or equal to 0.8 g/day (K/DOQI).

There was no statistically significant difference between treatments on mean creatinine clearance at day 28 (p=0.45) although there was a trend for an increase against baseline. Two patients in GHRH arm had reported 124.63% and 115.65% improvement against baseline.

There was no statistically significant difference between treatments on mean urea clearance at day 28 (p=0.11). The adjusted mean difference between GHRH and placebo arm was 2.28 ml/min/1.73 m² (95% CI −0.57 ml/min/1.73 m² to 5.13 ml/min/1.73 m²). Both groups exhibited reduction in urea clearance, however a smaller decrease was found in GHRH arms. In addition, two patients in the GHRH(1-29)NH₂ synthetic analog group had reported 123.99% and 103.49% improvement against baseline.

There was no statistically significant difference between treatments on mean GFR at day 28 (p=0.78). Both groups exhibited reduction in GFR, however a smaller decrease was found in GHRH arms. In addition, one patient in the GHRH(1-29)NH₂ synthetic analog group had reported 78.56% improvement against baseline. GFR is used to measure the level of kidney function and determine the stage of kidney disease. Many conditions can affect kidney functions. Some will lead to acute decline in GFR, others to a chronic decline. In our ITT population, all patients exhibited a reduction in GFR. The smaller reduction in the GHRH(1-29)NH₂ synthetic analog arm may demonstrate potential of the GHRH(1-29)NH₂ synthetic analog in slowing CKD progression.

There were no statistically significant differences between treatments on all EER measured at day 28. The adjusted mean difference between treatment groups for calcium (p=0.69), chloride (p=0.71), phosphate (p=0.59), potassium (p=0.36) and sodium (p=0.96) were −0.002 mmol/kg/24 h (95% CI −0.015 mmol/kg/24 h to 0.010 mmol/kg/24 h), −0.136 mmol/kg/24 h (95% CI −0.885 mmol/kg/24 h to 0.614), 0.020 mmol/kg/24 h (95% CI −0.055 mmol/kg/24 h to 0.094 mmol/kg/24 h), −0.086 mmol/kg/24 h (95% CI −0.277 mmol/kg/24 h to 0.105 mmol/kg/24 h), and −0.020 mmol/kg/24 h (95% CI −0.767 mmol/kg/24 h to 0.726 mmol/kg/24 h) respectively.

There was no statistically significant difference between treatments on mean fractional Pi reabsorption rate at day 28 (p=0.47). The adjusted mean difference between GHRH and placebo arm was −0.000059 mmol/ml (95% CI −0.000228 mmol/ml to 0.000110 mmol/ml).

There was no statistically significant difference between treatments on mean sodium (p=0.93) and calcium excretion (p=0.67) at day 28. The adjusted mean difference between GHRH and placebo arm for sodium and calcium excretion were −1.18 mmol/m²/24 h (95% CI −28.39 mmol/m²/24 h to 26.02 mmol/m²/24 h) and −0.10 mmol/m²/24 h (95% CI −0.56 mmol/m²/24 h to 0.36 mmol/m²/24 h).

The GH/IFGF-1 system is present in the kidney and is important to kidney structure and function. GHRH and GH receptors, IGF-1 and IGF-II, as well as IGF-1 and IGF-II receptors and IGFBPs are normally expressed in the adult (rat) kidney, suggesting a role for GH and IGF in regulating kidney function. GH may increase renal hemodynamics and filtration rate, and glomerular filtration rate (GFR) and renal plasma flow rates are elevated in patients with acromegaly. GH increases GFR with a delay of many hours up to a day, consistent with induction of IGF-1 synthesis. IGF-1 increases GFR acutely when given pharmacologically. Endogenous IGF-1 may contribute to the physiologic regulation of GFR.

Creatinine clearance, urea clearance and GFR were all sustained at Day 28 compared with baseline in the GHRH(1-29)NH₂ synthetic analog group but fell in the placebo group, although the differences were not statistically significant. These findings indicate that one-month treatment was too short to demonstrate potential beneficial effect of the GHRH(1-29)NH₂ synthetic analog on renal function in these subjects with progressive chronic renal failure. Since a trend towards slowing down of deterioration of kidney function was observed in the GHRH(1-29)NH₂ synthetic analog group while the disease progressed in the placebo group might indicate that longer duration treatment could have a clinically useful effect.

### Spontaneous Nutrient Intake: Dietary Assessment

Dietary assessment was performed at baseline and day 28. Subjects were asked to write down everything they eat and drink throughout the three days preceding the visit. Local dietarian calculated the protein and energy intake. The energy intake was 172 kcal/day lower in GHRH group but the difference was no statistically significant (p=0.21). The mean energy intake (kcal/day) was, in GHRH group at baseline, 1654 and on day 28 1533 while in placebo it was 1746 at baseline and 1726 on day 28.

Also the protein intake was lower in GHRH group, on average 12 grams/day, but not statistically significantly (p=0.11).

The mean protein intake (gram/day) was in GHRH group at baseline 62.46 and on day 28 58.49 while in placebo it was 62.01 at baseline and 65.99 on day 28.

There were no substantial changes from baseline or statistically significant differences between the groups in protein or energy intake, although both of these variables tended be lower at Day 28 in the GHRH(1-29)NH₂ synthetic analog group compared with the placebo group.

The number and nature of AEs was consistent with subjects with chronic renal failure. Although the number of AEs reported by subjects in the GHRH(1-29)NH₂ synthetic analog group was higher than the number reported by subjects in the placebo group (70 vs. 58), there was no clear pattern to distinguish the treatments from each other in safety terms. This might be partly due to the relatively low number of subjects in the study but, overall, the 2 groups were generally balanced with respect to the nature and incidence of AEs.

### SUMMARY OF RESULTS

### Efficacy Results:

### Protein Turnover

In the ITT population, mean leucine oxidation normalized with body weight at baseline was 7.74 μmol/kg.hr in the GHRH(1-29)NH₂ synthetic analog group and 9.83 μmol/kg.hr in the placebo group. Mean and median leucine oxidation was higher at Day 28 compared with baseline in the GHRH group but was lower or with very small change in the placebo group: mean leucine oxidation increased by 2.38 μmol/kg.hr in the GHRH(1-29)NH₂ synthetic analog group and by 0.84 μmol/kg.hr in the placebo group. However, the difference between the groups was not statistically significant (p=0.750). The adjusted mean difference between the groups was 1.1 μmol/kg.hr (95% CI −6.0; 8.2). The findings of the PP (Leucine Kinetic) population supported the findings of the ITT population. Mean leucine oxidation values both at baseline and at Day 28 in both treatment groups were not indicative of net catabolism.

When a non-parametric analysis method was applied to the data, a statistically significant difference was detected between the groups (p=0.009 ITT population; p=0.040 PP [Leucine Kinetic] population).

### Other Leucine Kinetics Parameters (Protein Turnover)

At Day 28, mean leucine flux normalized with body weight was 1.15 μmol/kg.hr higher than the baseline value of 101.46 μmol/kg.hr in the GHRH(1-29)NH₂ synthetic analog group but was 3.19 μmol/kg.hr lower than the baseline value of 94.01 μmol/kg.hr in the placebo group. The adjusted mean difference (95% CI) was 7.5 μmol/kg.hr (−12.0; 27.0) and was not statistically significant (p=0.431).

At Day 28, mean protein synthesis normalized with body weight was 1.22 μmol/kg.hr lower than the baseline value of 93.71 μmol/kg.hr in the GHRH(1-29)NH₂ synthetic analog group and was 4.03 μmol/kg.hr lower than the baseline value of 84.19 μmol/kg.hr in the placebo group. The adjusted mean difference (95% CI) was 6.9 μmol/kg.hr (−10.9; 24.7) and was not statistically significant (p=0.431).

At Day 28, mean protein breakdown normalized with body weight was 1.16 μmol/kg.hr higher than the baseline value of 97.45 μmol/kg.hr in the GHRH(1-29)NH₂ synthetic analog group but was 3.19 μmol/kg.hr lower than the baseline value of 90.01 μmol/kg.hr in the placebo group. The adjusted mean difference (95% CI) was 7.5 μmol/kg.hr (−11.9; 27.0) and was not statistically significant (p=0.430).

Median oxidation of flux normalized with body weight was 3.8% higher at Day 28 than the baseline value in the GHRH(1-29)NH₂ synthetic analog group but was 1.9% lower than the baseline value in the placebo group. The adjusted mean difference (95% CI) was 1.48% (0.95; 2.30) and was not statistically significant (p=0.077).

Median protein synthesis of flux normalized with body weight was 3.8% lower than the baseline value in the GHRH(1-29)NH₂ synthetic analog group but was 1.9% higher than the baseline value in the placebo group. The difference for the dose group comparisons was not statistically significant (p=0.061).

The findings of the PP (Leucine Kinetics) population supported the findings of the ITT population.

### SGA

According to the SGA results, subjects were better nourished in the GHRH(1-29)NH₂ synthetic analog group at Day 28, than in the placebo group (p=0.0083). In particular, the number of well nourished subjects in the GHRH(1-29)NH₂ synthetic analog group was 3 (23.1%) at baseline and 9 (75.0%) at Day 28, whereas in the placebo group, 3 subjects were well nourished both at baseline (20.0%) and at Day 28 (21.4%).

### Body Composition:

### 1. Anthropometry

On day 28, the subjects the GHRH(1-29)NH₂ synthetic analog group were, on average, 0.96 kg (95% CI 0.17; 1.75) heavier that the subjects in placebo group and the difference was statistically significant (p=0.019).

There were no statistically significant differences between the groups at Day 28 in any of the anthropometry measures.

Median fat mass decreased between baseline (15.8 kg) and Day 28 (15.6 kg) in the GHRH(1-29)NH₂ synthetic analog group but increased in the placebo group (14.4 kg at baseline, 14.9 kg at Day 28). However, median percent fat mass increased in both groups (from 25.4% at baseline to 25.9% at Day 28 in the GHRH(1-29)NH₂ synthetic analog group and from 23.7% at baseline to 24.6% at Day 28 in the placebo group).

Mean fat free mass decreased in both groups, from 46.64 kg at baseline to 46.20 kg at Day 28 in the GHRH(1-29)NH₂ synthetic analog group and from 49.32 kg at baseline to 49.46 kg at Day 28 in the placebo group.

Arm circumference and subscapular skinfold thickness increased between baseline and Day 28 in both groups. Biceps skinfold thickness decreased between baseline and Day 28 in the GHRH(1-29)NH₂ synthetic analog group and increased in the placebo group, whereas both triceps and suprailiacal skinfold thicknesses increased between baseline and Day 28 in the GHRH(1-29)NH₂ synthetic analog group and decreased in the placebo group.

### 2. Bioimpedance

Median fat mass (kg), measured by bioimpedance, was lower in the GHRH(1-29)NH₂ synthetic analog group at all visits and the differences were statistically significant at every visit except Day 14. The estimations of fat mass (%) and free fat mass (kg) were similar to fat mass. There were also statistically significant differences between the groups in extracelluar cell mass, extracellular water, intracellular water and total body water at Day 28.

### 3. DEXA Scan

Mean fat mass measured by DEXA scan on Day 28, in the GHRH(1-29)NH₂ synthetic analog group was 84% of the mean fat mass in the placebo group (95% CI; 0.72-0.98, p=0.029). Similarly mean fat mass at Day 28 for the GHRH(1-29)NH₂ synthetic analog group was 83% of the mean in placebo group (95% CI; 71%-96%, p=0.014). Statistically significant differences were also observed in mean fat free mass (kg), which was 2.9 kg higher in the GHRH(1-29)NH₂ synthetic analog group compared with the placebo group (95% CI; 0.5-5.4 kg, p=0.022).

Total body bone mineral content increased between baseline and Day 28 in the GHRH(1-29)NH₂ synthetic analog group and decreased in the placebo group. The increase in bone mineral content occurred in the trunk: mean bone mineral content in the trunk was 0.0699 kg at baseline and 0.718 kg at Day 28 in the GHRH(1-29)NH₂ synthetic analog group compared with 0.700 kg both at baseline and at Day 28 in the placebo group.

### Biochemical Markers of Nutritional and Metabolic State

Median fasting insulin concentrations were consistently higher for the GHRH(1-29)NH₂ synthetic analog group compared with the placebo group (95% CI; CI: 1.131-2.829, p=0.015). At Day 28, median fasting insulin was 12.1 μunits/mL in the GHRH(1-29)NH₂ synthetic analog group and 7.0 μunits/mL in the placebo group. Similarly, fasting cholesterol concentrations were lower for the GHRH(1-29)NH₂ synthetic analog group (Day 28 value 4.69 mmol/L) compared with the placebo group (Day 28 value 5.84 mmol/L: 95% CI; −1.02-−0.13, p=0.014). Statistically significant differences between groups were observed in fasting insulin and fasting cholesterol.

There were no statistically significant differences between the 2 groups in albumin, pre-albumin, transferrin, HbA_{1c}, fasting glucose, or fasting triglycerides.

### GH Profile

On Day 28, the second component GH half-life was longer for the GHRH(1-29)NH₂ synthetic analog group compared with the placebo group (95% CI; 0.6-9.0, p=0.029). Statistically significant differences between the groups were also observed for the GHRH(1-29)NH₂ synthetic analog group compared with the placebo group in the following: time from onset to peak of GH burst was shorter (95% CI; −9.0-−0.1, p=0.048); basal GH secretion rate was greater (95% CI; 1.34-4.79, p=0.007); pulsatile GH secretion rate was greater (95% CI; 1.15-6.89, p=0.026); total GH secretion rate was greater (95% CI; 1.23-5.99, p=0.016); and mass per pulse was greater (95% CI; 7.2-45.8, p=0.009).

### Insulin-Like Growth Factor

Median IGF-1 concentrations in the GHRH(1-29)NH₂ synthetic analog group were statistically significantly higher compared with those in the placebo group on every occasion of measurement. Mean IGFBP-1 concentrations in the placebo group were statistically significantly greater on Day 28 compared with the GHRH-group (mean difference 28.40 ng/mL; 95% CI; −50.52-6.29; p=0.014). Differences between the 2 groups on Day 28 in IGFBP-3 concentrations were not statistically significantly different.

### Fat Regulation

There were no statistically significant differences between the 2 groups in leptin concentrations (ratio of mean difference at Day 28 0.866 ng/mL) or in median leptin concentrations on any occasion of measurement.

### Sex Hormones

There was little change in testosterone in either group. However, mean oestradiol values at Day 28 were almost half those at baseline in the GHRH(1-29)NH₂ synthetic analog group (30.400 pg/mL at baseline, 16.281 pg/mL at Day 28) compared with only a small decrease in placebo-treated subjects (31.983 pg/mL at baseline, 28.500 pg/mL at Day 28).

### 24-Hour Urine Samples: Kidney Function

Median creatinine clearance at baseline was higher in the GHRH(1-29)NH₂ synthetic analog group (24.92 mL/min/1.73 m⁻²) compared with the placebo group (19.93 mL/min/1.73 m⁻²). Creatinine clearance was sustained at Day 28 in the GHRH(1-29)NH₂ synthetic analog group but was lower in the placebo group (23.78 mL/min/1.73 m⁻² GHRH group; 15.81 mL/min/1.73 m⁻² placebo group) but the difference was not statistically significant (95% CI; 0.83-1.51, p=0.451).

Mean urea clearance rate was sustained between baseline and Day 28 in the GHRH(1-29)NH₂ synthetic 2-analog group (11.86 mL/min/1.73 m⁻¹ at baseline; 11.24 mL/min/1.73 m⁻² on Day 28) but fell in the placebo group (11.77 mL/min/1.73 m⁻² at baseline and 8.960 mL/min/1.73 m⁻² on Day 28), but the difference was not statistically significant (95% CI; −0.57-5.13, p=0.111).

Median GFR was sustained between baseline and Day 28 in the GHRH group (19.37 mL/min/1.73 m⁻² at baseline and 18.23 mL/min/1.73 m⁻² on Day 28) but fell in the placebo group (18.10 mL/min/1.73 m⁻² at baseline and 13.54 mL/min/1.73 m⁻² on Day 28), but the difference was not statistically significant (p=0.777).

These results demonstrate that deterioration or renal function continued in the Placebo group but slowed down in the GHRH(1-29)NH₂ synthetic analog group suggesting a reno-protective effect of the GHRH(1-29)NH₂ synthetic analog.

There were no statistically significant differences between the groups in 24-hour urinary electrolyte excretion.

### Dietary Assessment

There were no substantial changes from baseline or statistically significant differences between the groups in protein or energy intake. Mean protein intake was 62.5 g/day at baseline and 58.5 g/day on Day 28 for the GHRH(1-29)NH₂ synthetic analog group, and 62.0 g/day at baseline and 66.0 g/day on Day 28 for the placebo group. Mean energy intake was 1654.5 kcal/day at baseline and 1532.7 kcal/day on Day 28 for the GHRH(1-29)NH₂ synthetic analog group, and 1746.0 kcal/day at baseline and 1725.5 kcal/day on Day 28 for the placebo group.

### CONCLUSIONS

In both ITT and PP populations, there was no statistically significant difference between treatment groups in total body protein turnover. The values both at baseline and after 28 days of treatment were not indicative of catabolism.

In the ITT population, a 4-week treatment with the GHRH(1-29)NH₂ synthetic analog induced in malnourished patients with CKD the following statistically significant changes in comparison to placebo:
A 393% increase in mean 24-hour integrated GH secretion rate as compared to 22% increase in the placebo group (24 h AUC (μU/ml×min). GHRH(1-29)NH₂ synthetic analog treatment did not affect the natural pulsatile rhythm of endogenous GH secretion.
A 104% increase in median total circulating IGF-1 and mean 26.7% decrease in IGFBP-1 concentration, indicating availability of more free circulating IGF-1 for action, as compared to 3.5% increase in IGF-1 and 2.4% increase in IGFBP-1 in the placebo.
An increase from baseline in median FFM (3.3 kg by BIA and 1.6 kg by DEXA) and decrease in median FM (1.8 kg by BIA and kg 0.4 kg by DEXA) in all except one (11/12) patients in GHRH group on day 28 while there was a decrease in median FFM (0.8 kg by BIA and by 0.7 kg DEXA) and an increase in median FM (1.3 kg by BIA and 0.0 kg by DEXA) in the placebo arm. The increase in FFM was most pronounced in the leg and trunk regions.
A median increase from baseline of 1.8 kg in body cell mass (BCM) (in comparison with decrease of 0.6 kg in the placebo group), the most metabolically active component of LBM, that has been reported to correlate positively with the decrease in wasting and an increase in muscle strength, regain of function and reduction in morbidity.
A GH-mediated increase in median extracellular water (ECW) by 1.9 L and intracellular water (ICW) by 1.2 L while the ratio of median ECW/ICW (1.2) on day 28 remained relatively unchanged from that at baseline.
An increase in basal metabolic rate (BMR) as measured by BIA.
Patient reported subjective improvement in nutritional and well-being status. Out of 9 mild to moderate malnourished patients in the GHRH group 6 become well-nourished after 28 days of therapy while all 9 mild to moderate malnourished patients in the placebo group remained malnourished.

There were no statistically significant differences between treatments on mean serum albumin (p=0.43), pre-albumin (p−0.076), and transferrin (p=0.32) at day 28.

Although there was no statistically significant difference between treatments on GFR, mean creatinine and urea clearance at day 28, there was a trend for an increase against baseline in the GHRH(1-29)NH₂ synthetic analog group, indicating slow down in disease progression compared to placebo. Two patients in the GHRH arm had reported over 100% increase in creatinine and urea clearance.

There were no statistically significant differences between treatments on all electrolyte excretion rates (EER) measured at day 28 for calcium, chloride, phosphate, potassium and sodium.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description to the invention. Changes may be made in the elements described herein without departing from the spirit and scope of the invention as described in the following claims. In addition, it is to be understood that features described herein independently may, in certain embodiments, be combined.

What is claimed is:

1. A method of treating acute or chronic kidney disease including acute or chronic renal failure in a subject comprising administering to a subject who would benefit from such treatment a pharmaceutical dosage form comprising a GHRH analog, a functional derivative of said analog, or a pharmaceutically acceptable salt thereof, and wherein the GHRH analog has the amino acid sequence Tyr-A2-Asp-Ala-Ile-Phe-Thr-A8-A9-A10-Arg-Lys-Val-Leu-A15-Gln-Leu-Ser-Ala-Arg-A21-A22-Leu-Gln-Asp-Ile-Met-Ser-Arg-A30-NH₂, where:
A2 is Ala or D-Ala;
A8 is Asn, D-Asn or Ala;
A9 is Ser or Ala;
A10 is Tyr or D-Tyr;
A15 is Gly, Ala or D-Ala;
A21 is Lys or D-Lys;
A22 is Leu, D-Leu, Lys or Ala; and
A30 is a bond or any amino acid sequence of 1 up to 15 residues;
wherein said analogue comprises at least one of the above amino acid substitutions in comparison with the amino acid sequence of the native form of hGHRH1-29.

2. The method in accordance with claim 1, wherein the pharmaceutical dosage form is administered to the subject at least once per day.

3. The method in accordance with claim 1, wherein the pharmaceutical dosage form is administered to the subject at least twice per day.

4. The method in accordance with claim 1, wherein the pharmaceutical dosage form is administered to the subject for about 30 days. The method in accordance with claim 1, wherein the pharmaceutical dosage form comprises up to about 10 mg of the GHRH analog. The method in accordance with claim 1, wherein the GHRH analog is the compound where A2 is D-Ala, A8 is Asn, A9 is Ser; A10 is D-Tyr; A15 is D-Ala, A21 is Lys; A22 is Lys and A30 is a bond.

7. The method in accordance with claim 1, wherein the GHRH analog is the compound where A2 is D-Ala, A8 is Ala, A9 is Ser, A10 is Tyr, A15 is Ala, A21 is Lys, A22 is Lys, and A30 is a bond.

8. The method in accordance with claim 1, wherein the GHRH analog is the compound where A2 is Ala, A8 is Ala, A9 is Ala, A10 is Tyr, A15 is Ala, A21 is Lys, A22 is Ala and A30 is a bond.

9. The method in accordance with claim 1, wherein the GHRH analog is the compound where A2 is D-Ala, A8 is Asn, A9 is Ser, A10 is D-Tyr, A15 is Gly, A21 is Lys, A22 is Lys and A30 is a bond.

10. The method in accordance with claim 1, wherein the GHRH analog is the compound where A2 is D-Ala, A8 is Ala, A9 is Ser, A10 is D-Tyr, A15 is Ala, A21 is D-Lys, A22 is Lys and A30 is a bond.

11. A method of treating wasting or cachexia indications in a subject comprising administering to a subject who would benefit from such treatment a pharmaceutical dosage form comprising a GHRH analog, a functional derivative of said analog, or a pharmaceutically acceptable salt thereof, and wherein the GHRH analog has the amino acid sequence Tyr-A2-Asp-Ala-Ile-Phe-Thr-A8-A9-A10-Arg-Lys-Val-Leu-A15-Gln-Leu-Ser-Ala-Arg-A21-A22-Leu-Gln-Asp-Ile-Met-Ser-Arg-A30-NH₂, where:
A2 is Ala or D-Ala;
A8 is Asn, D-Asn or Ala;
A9 is Ser or Ala;
A10 is Tyr or D-Tyr;
A15 is Gly, Ala or D-Ala;
A21 is Lys or D-Lys;
A22 is Leu, D-Leu, Lys or Ala; and
A30 is a bond or any amino acid sequence of 1 up to 15 residues;
wherein said analogue comprises at least one of the above amino acid substitutions in comparison with the amino acid sequence of the native form of hGHRH1-29.

12. The method in accordance with claim 11, wherein the pharmaceutical dosage form is administered to the subject at least once per day.

13. The method in accordance with claim 11, wherein the pharmaceutical dosage form is administered to the subject at least twice per day.

14. The method in accordance with claim 11, wherein the pharmaceutical dosage form is administered to the subject for about 30 days. The method in accordance with claim 11, wherein the pharmaceutical dosage form comprises up to about 10 mg of the GHRH analog. The method in accordance with claim 11, wherein the GHRH analog is the compound where A2 is D-Ala, A8 is Asn, A9 is Ser; A10 is D-Tyr; A15 is D-Ala, A21 is Lys; A22 is Lys and A30 is a bond.

17. The method in accordance with claim 11, wherein the GHRH analog is the compound where A2 is D-Ala, A8 is Ala, A9 is Ser, A10 is Tyr, A15 is Ala, A21 is Lys, A22 is Lys, and A30 is a bond.

18. The method in accordance with claim 11, wherein the GHRH analog is the compound where A2 is Ala, A8 is Ala, A9 is Ala, A10 is Tyr, A15 is Ala, A21 is Lys, A22 is Ala and A30 is a bond.

19. The method in accordance with claim 11, wherein the GHRH analog is the compound where A2 is D-Ala, A8 is Asn, A9 is Ser, A10 is D-Tyr, A15 is Gly, A21 is Lys, A22 is Lys and A30 is a bond.

20. The method in accordance with claim 11, wherein the GHRH analog is the compound where A2 is D-Ala, A8 is Ala, A9 is Ser, A10 is D-Tyr, A15 is Ala, A21 is D-Lys, A22 is Lys and A30 is a bond.

21. The method in accordance with claim 11, wherein the wasting or cachexia indications are associated with acute or chronic kidney disease.

22. The method in accordance with claim 11, wherein the wasting or cachexia indications are associated with AIDS.

23. The method in accordance with claim 11, wherein the wasting or cachexia indications are associated with cancer.
